(19) United States
(12) Patent Application Publication
(10) Pub. No.: US 2025/0339160 A1
(43) Pub. Date: Nov. 6, 2025

(54) METHODS FOR REMOVING CLOT MATERIAL FROM A PATIENT

(71) Applicant: Inquis Medical, Inc., Menlo Park, CA (US)

(72) Inventors: William Jason FOX, San Mateo, CA (US); Vahid SAADAT, Atherton, CA (US); Max Niklas ROTHE, Atherton, CA (US); Richard CHILDS, Atherton, CA (US); Michael PARE, Menlo Park, CA (US); Paul NAULEAU, Menlo Park, CA (US); Koji KIZUKA, Atherton, CA (US); Steve MILLER, Menlo Park, CA (US); Jared ROSEMAN, Atherton, CA (US)

(21) Appl. No.: 19/200,626

(22) Filed: May 6, 2025

Related U.S. Application Data
(60) Provisional application No. 63/643,398, filed on May 6, 2024.
(60) Provisional application No. 63/653,191, filed on May 29, 2024.
(60) Provisional application No. 63/653,194, filed on May 29, 2024.
(60) Provisional application No. 63/667,119, filed on Jul. 2, 2024.
(60) Provisional application No. 63/715,494, filed on Nov. 1, 2024.
(60) Provisional application No. 63/740,312, filed on Dec. 30, 2024.
(60) Provisional application No. 63/768,167, filed on Mar. 6, 2025.

(51) Int. Cl. A61B 17/22; A61B 17/00

(52) U.S. Cl. CPC A61B 17/22; A61B 2017/00026; A61B 2017/00561; A61B 2017/22079; A61B 2217/005

(57) ABSTRACT

Clot material may be removed from a patient and separated from blood so that the blood may be collected using an efficient dual-stroke technique. The methods described herein withdraw clot and blood from a patient during a first stoke of a pump at a first rate, filter the blood to remove clot, and drive the filtered blood into a collection chamber at a second rate that is different from the first rate. The first rate may be optimized to disrupt and remove clot, while the second rate may be optimized to move the blood without damaging, e.g., lysing, the blood.

### RELATED APPLICATIONS

This application claim priority to each of the following U.S. provisional patent application No. 63/643,398 (titled “APPARATUS AND METHODS FOR REMOVAL OF OBSTRUCTIVE MATERIAL USING FLUIDIC DRIVEN ASPIRATION DEVICE”), filed on May 6, 2024; U.S. provisional patent application No. 63/653,191 (titled “APPARATUS AND METHODS FOR REMOVAL OF OBSTRUCTIVE MATERIAL USING FLUIDIC DRIVEN ASPIRATION DEVICE”), filed on May 29, 2024; U.S. provisional patent application No. 63/653,194 (titled “AUTOMATED THROMBECTOMY SYSTEM”), filed on May 29, 2024; U.S. provisional patent application No. 63/667,119 (titled “AUTOMATED THROMBECTOMY SYSTEM”), filed on Jul. 2, 2024; U.S. provisional patent application No. 63/715,494 (titled “AUTOMATED THROMBECTOMY SYSTEM”), filed on Nov. 1, 2024; U.S. provisional patent application No. 63/740,312 (titled “AUTOMATED THROMBECTOMY SYSTEM”) filed on Dec. 30, 2024; and U.S. provisional patent application No. 63/768,167 (titled “THROMBECTOMY APPARATUSES AND METHODS”) filed on Mar. 6, 2025. Each of these application is herein incorporated by reference in its entirety.

### BACKGROUND

Thrombectomy is the removal of blood clots from various parts of the human vasculature. The current state of the art in thrombectomy includes several types of systems, including manual aspiration with a syringe, aspiration via vacuum-pump and computerized valve control, and physical scraping/catching of clot with metal mesh devices. None of these devices include any sensing of tissue-type at the catheter tip, and only the manual syringe-based products allow for blood return to the patient.

Systems which include a vacuum chamber and a vacuum-valve to connect the aspiration lumen to the vacuum chamber suffer from hemolysis of the blood, which renders it unreturnable to the patient. This occurs when the blood is exposed to open vacuum/air for extended periods, which also causes the blood to foam and degas. Procedures with these types of systems tend to incur excessive blood loss and may be prematurely halted due to blood loss concerns.

Systems which require manual operation for clot extraction are ill-suited to peripheral thrombectomy procedures such as deep-vein thrombosis (DVT), where the amount of clot can be quite large and require many aspiration/clot extraction cycles to clear. This includes both manual aspiration systems as well as systems which physically scape/catch the clot for mechanical extraction. The physical scraping devices in particular require longer procedures and multiple passes of the device through the vessels to clear the clot.

While very effective, certain design aspects of currently available clot aspiration systems could be improved. For example, most current systems use either a syringe or a continuous pump for applying a negative pressure to a catheter lumen to aspirate the clot. Systems which require manual operation for clot extraction are ill-suited to peripheral thrombectomy procedures such as deep-vein thrombosis (DVT), where the amount of clot can be quite large and require many aspiration/clot extraction cycles to clear. Systems using a continuous pump can be difficult to control. It would thus be desirable to provide additional and alternative systems and apparatus for such negative pressure generation.

As another example, some current systems have inline filtration for separating the clot where the filters can clog, requiring the filter to be cleaned and the system to be primed before resuming aspiration. Such systems are also subject to air intrusion. Certain systems which include a vacuum chamber and a vacuum-valve to connect the aspiration lumen to the vacuum chamber suffer from hemolysis of the blood, which renders it unreturnable to the patient. This occurs when the blood is exposed to open vacuum/air for extended periods, which also causes the blood to foam and degas. Procedures with these types of systems tend to incur excessive blood loss and may be prematurely halted due to blood loss concerns. Thus, it would be desirable to provide aspiration and thrombectomy systems which facilitate detecting and removing clot and which reduce the risk of air intrusion.

### SUMMARY OF THE DISCLOSURE

This invention disclosure describes a system as well as various implementation options which use sensing at the tip of an aspiration catheter to inform or automate clot aspiration in a thrombectomy procedure using a pressure element (such as a syringe (or other motion-type vacuum element), automated syringe plunger control, and a set of one-way valves. This type of system benefits the user by powering and automating the clot extraction process, significantly reducing blood loss, removing clot more precisely, reducing effort by the user, and allowing any captured blood to be filtered and returned to the patient. This type of system improves procedural efficiency and ease of use and may be used even in patients for whom even moderate blood loss is a severe concern.

Described herein are apparatuses (systems, devices, etc.) and methods for making and using these systems for use in aspiration, particularly for clot aspiration in a thrombectomy procedure. These methods and apparatuses may use a pressure element (such as a syringe or other motion-type vacuum element), automated syringe plunger control, and a set of one-way valves. This may benefit the user by powering and automating the clot extraction process, significantly reducing blood loss, removing clot more precisely, reducing effort by the user, and allowing any captured blood to be filtered and returned to the patient. This type of system improves procedural efficiency and ease of use, and the system may be used even in patients for whom even moderate blood loss is a severe concern.

In particular, the fluidic drivers described herein can be useful with or without sensing and may allow for significantly easier and more effective single operator thrombectomy. These methods and apparatuses may also permit flowrates and aspiration power that are not possible using a manual syringe with stored vacuum without a substantial amount of user effort. These method and apparatuses may also allow movement of the blood out of a patient and back to the patient with minimal damage to the blood.

For example, described herein are aspiration apparatuses (e.g., devices, systems, etc.) for use with an aspiration catheter and a fluidic actuator that is configured to deliver a pressurized drive fluid. In some cases an aspiration apparatus (e.g., aspiration device) may include: an aspirator including an aspirator displacement element, an aspirator cylinder, and an aspirator port, wherein the aspirator port is configured to connect to an aspiration lumen of the aspiration catheter; and a fluidic driver including a driver displacement element, a driver cylinder, and at least a first fluid port configured to receive the pressurized drive fluid from the fluidic actuator, wherein the pressurized fluid causes the driver displacement element to translate in a first direction and wherein the aspirator displacement element is coupled to travel in tandem in the first direction with the driver displacement element to draw blood and clot through the aspirator port and into and from the aspiration cylinder, when a distal port of the aspiration catheter is in a patient blood vessel proximate clot.

Any of the fluidic aspirators may be configured to deliver at least a positive pressure drive fluid. The fluidic aspirator may be configured to deliver at least a negative pressure drive fluid. The fluidic aspirator may be configured to deliver both a positive pressure drive fluid and a negative pressure drive fluid. In any of these apparatuses the fluidic driver may further include a second fluid port configured to receive the pressurized drive fluid from the fluidic actuator, wherein the pressurized fluid causes the driver displacement element to translate in a second direction and wherein the aspirator displacement element is coupled to travel in tandem in the second direction with the driver displacement element eject blood and clot from the aspirator port. The fluidic driver may further include a bias (e.g., biasing spring) coupled to one side of the driver displaceable element, wherein said biasing spring is configured to counteract translation of the displacement element caused by the pressurized fluid.

In some examples the aspirator and fluidic driver are arranged in tandem. The aspirator and fluidic driver may be disposed in a common housing. For example, the common housing may comprise a cylinder having an internal wall separating the aspirator and fluidic driver. The aspirator and fluidic driver may be arranged in parallel.

In some examples the aspirator and fluidic driver may comprise separate housings and wherein the driver displacement element and the aspiration displacement element are joined by a coupling member disposed between the separate housings.

At least one of the displacement elements of the aspirator and the fluidic driver may comprise a piston. The displacement elements of the aspirator and the fluidic driver may each comprise a piston. The pistons may be configured to reciprocate in their respective cylinders with low friction. In some examples at least one of the displacement elements of the aspirator and the fluidic driver may comprise a diaphragm. The displacement elements of the aspirator and the fluidic driver may each comprise a diaphragm. In some cases the aspirator comprises a syringe.

Any of these apparatuses may include the fluidic actuator. The fluidic actuator may comprise an aspiration controller.

The aspiration controller may be programmable. The aspiration controller may be configured to respond to real-time user input. In some cases the aspiration controller is configured to modify a length of travel of the driver displacement element to control one or more of flow volume and flow rate. The aspiration controller may be configured to modify a reciprocation rate of the driver displacement element to control one or more of flow volume and flow rate. In some cases the aspiration controller is configured to receive data from one or more sensors which measure pressure, electrical impedance, optical properties, flow rate, and/or flow volume. The aspiration controller may be configured to control one or more of length of travel and reciprocation rate of the driver displacement element to control one or more of flow volume and flow rate in response to the received data. The fluidic driver and fluidic actuator may comprise a pneumatic driver and a pneumatic actuator. The fluidic driver and fluidic actuator may comprise a hydraulic driver and a hydraulic actuator.

For example, a fluidic actuator configured to deliver a pressurized drive fluid to a fluidic driver coupled to an aspirator and an aspiration catheter may include: a source of pressurized fluid; means for selectively delivering the pressurized fluid to a first fluid port of the fluidic driver, wherein the fluidic driver includes a driver displacement element and a driver cylinder, wherein the driver displacement element is coupled to an aspirator displacement element of the aspirator, and wherein delivery of the pressurized fluid to the first fluid port causes the driver displacement element to move the aspirator displacement element in a first direction to draw blood and clot through an aspirator port and delivery of the pressurized fluid to the second fluid port causes the driver displacement element to move the aspirator displacement element in a second direction to eject blood and clot through the aspirator port, respectively, when a distal port of the aspiration catheter is in a patient blood vessel proximate clot.

As mentioned, the fluidic actuator may comprise an aspiration controller. The aspiration controller may be programmable. The aspiration controller may be configured to respond to real-time user input. The aspiration controller may be configured to modify a length of travel of the driver displacement element to control one or more of flow volume and flow rate. The aspiration controller may be configured to modify a reciprocation rate of the driver displacement element to control one or more of flow volume and flow rate. The aspiration controller may be configured to receive data from one or more sensors which measure pressure, electrical impedance, optical properties, flow rate, and/or flow volume. The aspiration controller may be configured to control one or more of length of travel and reciprocation rate of the driver displacement element to control one or more of flow volume and flow rate in response to the received data.

Also described herein are methods of using any of these apparatuses. For example, described herein are methods for aspirating clot from the vasculature of a patient. A method may include: positioning a distal port of an aspiration catheter proximate the clot in the patient's vasculature, wherein a proximal end of an aspiration lumen of the aspiration catheter is attached to an aspirator port of an aspirator including an aspirator displacement element and aspirator cylinder; and delivering a pressurized drive fluid to a first port of a fluidic driver comprising a driver displacement element and a driver cylinder, wherein the driver displacement element is coupled to the aspirator displacement element; wherein delivery of the pressurized fluid to the first port translates the driver displacement element and aspirator displacement element in a first direction which generates a negative pressure in the aspiration lumen to draw the clot and blood into the aspiration lumen through the distal port.

The pressurized fluid may be delivered to the first port without interruption until a predetermined amount of clot has been collected in the aspirator cylinder. Delivery of the pressurized fluid to the first port may be interrupted to vary a negative pressure in the aspiration lumen and at the port to enhance clot fragmentation and collection.

Any of these methods may include delivering the pressurized fluid to a second port of the fluidic driver to translate the driver displacement element and aspirator displacement element in a second direction to eject the clot and blood through the aspirator port. Any of these methods may include diverting the clot and ejected through the aspirator port to a collection receptacle. In some cases the methods include diverting the clot and blood ejected through the aspirator port to a filter to separate blood from clot and returning the separated blood to the patent. The pressurized fluid may be delivered alternately to the first and second ports of the fluidic driver to oscillate a travel direction of the aspirator displacement element to cause a reversing flow pattern of clot and blood through the distal port of an aspiration catheter. In some cases the pressurized drive fluid is delivered to the first and/or the second ports of the fluidic driver in a pattern which creates a pulsatile pressure profile at the distal port of the aspiration catheter to enhance clot fragmentation and collection. The flow of pressurized fluid delivered to the first port may be greater than the flow of pressurized fluid delivered to the second port so that there is a net accumulation of clot and blood in the aspirator cylinder.

Any of these methods may include adjusting a translation length of the aspirator displacement element in the aspiration cylinder to vary a flow volume or rate of blood and clot into or from the distal port. For example, the method may include adjusting a translation frequency of the aspirator displacement element in the aspiration cylinder to vary a flow volume or rate of blood and clot into or from the distal port.

Any of these methods may include receiving data from one or more sensors which measure pressure, electrical impedance, optical properties, flow rate, and/or flow volume. In some cases the method includes controlling one or more of length of travel and reciprocation rate of the driver cylinder to control one or more of flow volume and flow rate in response to the received data.

The pressurized fluid may comprise a gas or a liquid.

The disclosed technology provides systems, apparatus, and methods for aspirating clot from a patient's vasculature.

In at least some implementations, the disclosed technologies will provide “powered” aspiration of clot through a variety of aspiration catheters, including pulmonary, cardiac, peripheral, and neurological clot aspiration catheters. The power may be provided “fluidically,” including both pneumatically and hydraulically, typically using piston or other positive displacement pump mechanisms which are fluidically driven by electronically controlled In at least some implementations, the disclosed technologies will provide.

In at least some implementations, the disclosed technologies will provide for filtration and deaeration of the aspirated clot and blood, allowing the filtered blood to be returned to the patient. The filtration and deaeration technologies may be used in combination with any aspiration technologies, including but not limited to the powered aspiration technologies disclosed herein.

The disclosed technologies and implementations may optionally use sensing at the tip of an aspiration catheter to inform or automate clot aspiration in a thrombectomy procedure using a pressure element (such as syringe or other motion-type vacuum element), automated syringe plunger control, and a set of one-way valves. This type of system benefits the user by powering and automating the clot extraction process, significantly reducing blood loss, removing clot more precisely, reducing effort by the user, and allowing any captured blood to be filtered and returned to the patient. This type of system improves procedural efficiency and ease of use and may be used even in patients for whom even moderate blood loss is a severe concern.

In a first aspect, the disclosed technology provides an aspiration device configured for use with (a) an aspiration catheter and (b) a fluidic actuator configured to deliver a pressurized drive fluid. The aspiration device comprises an aspirator and a fluidic driver. The aspirator includes an aspirator displacement element, an aspirator cylinder, and an aspirator port, where aspirator port is configured to be connected to an aspiration lumen of the aspiration catheter. The fluidic driver includes a driver displacement element, a driver cylinder, and at least a first fluid port configured to receive the pressurized drive fluid from the fluidic actuator, where the pressurized fluid causes the driver displacement element to translate in a first direction and where the aspirator displacement element is coupled to travel in tandem in the first direction with the driver displacement element to draw blood and clot through the aspirator port and into and from the aspiration cylinder, when a distal port of the aspiration catheter is in a patient blood vessel proximate clot.

In some instances, the fluidic aspirator is configured to deliver at least a positive pressure drive fluid.

In some instances, the fluidic aspirator is configured to deliver at least a negative pressure drive fluid.

In some instances, the fluidic aspirator is configured to deliver both a positive pressure drive fluid and a negative pressure drive fluid.

In some instances, the fluidic driver further includes a second fluid port configured to receive the pressurized drive fluid from the fluidic actuator, where the pressurized fluid delivered through the second port causes the driver displacement element to translate in a second direction and where the aspirator displacement element is coupled to travel in tandem in the second direction with the driver displacement element eject blood and clot from the aspirator port.

In other instances, the fluidic driver further includes a biasing spring coupled to one side of the driver displaceable element, wherein said biasing spring is configured to counteract translation of the displacement element caused by the pressurized fluid.

In some instances, the aspirator and the fluidic driver are arranged in parallel.

In other instances, the aspirator and the fluidic driver are arranged in tandem.

In some instances, the aspirator and the fluidic driver are disposed in a common housing. For example, the common housing may comprise a cylinder having an internal wall separating the aspirator and fluidic driver.

In some instances, the aspirator and fluidic driver comprise separate housings where the driver displacement element and the aspiration displacement element may be joined by a coupling member disposed between the separate housings.

In some instances, at least one of the displacement elements of the aspirator and the fluidic driver comprises a piston.

In some instances, the displacement elements of the aspirator and the fluidic driver each comprise a piston where the pistons may be configured to reciprocate in their respective cylinders with low friction.

In some instances, at least one of the displacement elements of the aspirator and the fluidic driver comprises a diaphragm.

In some instances, the displacement elements of the aspirator and the fluidic driver each comprise a diaphragm.

In some instances, the aspirator comprises a syringe.

In some instances, the aspiration devices may further comprise the fluidic actuator.

In some instances, the fluidic actuator may comprise an aspiration controller.

In some instances, the aspiration controller may be programmable.

In other instances, the aspiration controller may be configured to respond to real-time user input.

In some instances, the aspiration controller may be configured to modify a length of travel of the driver displacement element to control one or more of flow volume and flow rate.

In some instances, the aspiration controller may be configured to modify a reciprocation rate of the driver displacement element to control one or more of flow volume and flow rate.

In some instances, the aspiration controller may be configured to receive data from one or more sensors which measure pressure, electrical impedance, optical properties, flow rate, and/or flow volume. In such instances, the aspiration controller may be configured to control one or more of length of travel and reciprocation rate of the driver displacement element to control one or more of flow volume and flow rate in response to the received data.

In some instances, the fluidic driver and fluidic actuator may comprise a pneumatic driver and a pneumatic actuator.

In other instances, the fluidic driver and fluidic actuator may comprise a hydraulic driver and a hydraulic actuator.

In a second aspect, the disclosed technology provides a fluidic actuator configured to deliver a pressurized drive fluid to a fluidic driver coupled to an aspirator and an aspiration catheter. The fluidic actuator comprises a source of pressurized fluid and a valve arrangement or other means for selectively delivering the pressurized fluid to a first fluid port of the fluidic driver. The fluidic driver typically includes a driver displacement element and a driver cylinder, where the driver displacement element is coupled to an aspirator displacement element of the aspirator. Delivery of the pressurized fluid to the first fluid port causes the driver displacement element to move the aspirator displacement element in a first direction to draw blood and clot through an aspirator port, and delivery of the pressurized fluid to the second fluid port causes the driver displacement element to move the aspirator displacement element in a second direction to eject blood and clot through the aspirator port, respectively, when a distal port of the aspiration catheter is in a patient blood vessel proximate clot.

In some instances, the fluidic actuator comprises an aspiration controller. In such instances, the aspiration controller may be either programmable, configured to respond to real-time user input, or some combination thereof.

In some instances, the aspiration controller may be configured to modify a length of travel of the driver displacement element to control one or more of flow volume and flow rate.

In some instances, the aspiration controller may be configured to modify a reciprocation rate of the driver displacement element to control one or more of flow volume and flow rate.

In some instances, the aspiration controller may be configured to receive data from one or more sensors which measure pressure, electrical impedance, optical properties, flow rate, and/or flow volume. In such instances, the aspiration controller may. e configured to control one or more of length of travel and reciprocation rate of the driver displacement element to control one or more of flow volume and flow rate in response to the received data.

In a third aspect, the disclosed technology provides a method for aspirating clot from the vasculature of a patient. The method typically comprises positioning a distal port of an aspiration catheter proximate the clot in the patient's vasculature while a proximal end of an aspiration lumen of the aspiration catheter is attached to an aspirator port of an aspirator. The aspirator includes a displacement element and aspirator cylinder, arranged for example as a syringe, which is connected to a fluidic driver, and which can be used to generate a negative pressure (a full or partial vacuum) to draw blood and clot into the aspiration catheter. The fluidic driver comprises a driver displacement element and a driver cylinder, wherein the driver displacement element is coupled to the displacement element in the aspirator a pressurized drive fluid to can be delivered to a first port of the fluidic driver to cause both the fluidic driver and the aspirator displacement elements the to translate in a first direction which generates a negative pressure in the aspiration lumen to draw the clot and blood into the aspiration lumen through the distal port.

In some instances, the pressurized fluid is delivered from the fluidic aspirator to the first port without interruption until a predetermined amount of clot has been collected in the aspirator cylinder.

In other instances, the delivery of the pressurized fluid from the fluidic aspirator to the first port is interrupted to vary a negative pressure in the aspiration lumen and at the port to enhance clot fragmentation and collection.

In some instances, the disclosed methods further comprise delivering the pressurized fluid the fluidic aspirator to a second port of the fluidic driver to translate the driver displacement element and aspirator displacement element in a second direction to eject the clot and blood through the aspirator port. In some such instances, the clot and blood ejected through the aspirator port may be diverted to a collection receptacle. In other such instances, the clot and blood ejected through the aspirator port may be diverted to a filter to separate blood from clot and return the separated blood to the patent.

In some instances, the pressurized fluid may be delivered alternately to the first and second ports of the fluidic driver to oscillate a travel direction of the aspirator displacement element to cause a reversing flow pattern of clot and blood through the distal port of an aspiration catheter. In some such instances, the pressurized drive fluid is delivered to the first and/or the second ports of the fluidic driver in a pattern which creates a pulsatile pressure profile at the distal port of the aspiration catheter to enhance clot fragmentation and collection. Typically, the flow of pressurized fluid delivered to the first port is greater than the flow of pressurized fluid delivered to the second port so that there is a net accumulation of clot and blood in the aspirator cylinder.

In some instances, these methods may further comprise adjusting a translation length of the aspirator displacement element in the aspiration cylinder to vary a flow volume or rate of blood and clot into or from the distal port.

In some instances, these methods may further comprise adjusting a translation frequency of the aspirator displacement element in the aspiration cylinder to vary a flow volume or rate of blood and clot into or from the distal port.

In some instances, these methods may further comprise receiving data from one or more sensors which measure pressure, electrical impedance, optical properties, flow rate, and/or flow volume.

In some instances, these methods may further comprise controlling one or more of length of travel and reciprocation rate of the driver cylinder to control one or more of flow volume and flow rate in response to the received data.

In some instances, the pressurized fluid may comprise a gas.

In some instances, the pressurized fluid may comprise a liquid.

In a fourth aspect, the disclosed technology provides blood filtering apparatus comprising a filter chamber and a deaeration chamber. The filter chamber includes a filter element that divides the filter chamber into an upper portion having a blood inlet and a lower portion, and the filter element is configured to separate clot from a pressurized flow of blood and clot entering the upper portion through the blood inlet and pass blood substantially free from clot into the lower portion. The deaeration chamber includes a lower portion having a blood outlet and an upper portion including a gas vent, where the lower portion of the deaeration chamber is configured to receive filtered, pressurized blood from the lower portion of the filter chamber and to separate gas present in said filtered, pressurized blood and the upper portion is configured to allow the separated gas to pass out through the gas vent. A one-way valve is configured to allow pressurized, filtered blood in the lower portion of the filter chamber to flow the lower portion of the deaeration chamber and to prevent a reverse flow of blood from the deaeration chamber to the filter chamber.

In some instances, at least a portion of a top of the filter chamber is sufficiently transparent to allow viewing of clot collected on an upper surface of the filter. Typically, the top is removable to allow removal and return of at least an upper portion of the filter element to permit cleaning of the clot and a means for cleaning a lower surface of the top of the filter chamber, such as a rotatable wiper blade, may be provided to remove adherent clot and improve viewing.

In some instances, at least a portion of the filter element may be removable from the filter chamber to allow clot to be removed from an upper surface thereof. For example, the filter element may comprise an upper strainer component and a lower microporous filter component, where the upper strainer component may be removably positioned over the lower microporous filter component. In specific examples, the upper strainer component may be separable from the lower microporous filter component and the lower microporous filter component may be fixedly positioned within the filter chamber.

In some instances, the gas vent on the upper portion of the deaeration chamber may comprise a gas vent valve configured to close when the deaeration chamber fills with pressurized blood and to open when separated gas collects in the upper portion of the deaeration chamber. In specific instances, the gas vent valve may comprise a float valve which is buoyed by blood in the deaeration chamber and opened by gas collecting in the upper portion of the deaeration chamber above the float valve. For example, the float valve may comprise a resilient seal on an upper surface thereof, where the resilient seal engages a vent port on an upper wall of the deaeration chamber and the float valve may ride on rails disposed on an inner wall of the deaeration chamber.

In some instances, the apparatus may further comprise a vertical support tube having a deflector on an upper end thereof, where the vertical support tube may be configured to receive the pressurized, filtered blood entering the lower portion of the deaeration chamber and to pass the blood upwardly to engage a lower surface of the deflector which redirects the blood downwardly and allows gas to separate and rise upwardly into the upper portion of the deaeration chamber.

In some instances, the apparatus may further comprise a cutoff valve at the blood outlet of the deaeration chamber, wherein the cutoff valve is configured to close the blood outlet if blood in the deaeration chamber falls below a minimum level. For example, the cutoff valve may comprise a ball valve.

In some instances, the apparatus may further comprise a pressure source connectable to a proximal end of an aspiration catheter and to the lower portion of the filter chamber, wherein said pressure source is configured to generate a negative pressure to draw blood and clot from the aspiration catheter and to generate a positive pressure to transfer the drawn blood and clot to the lower portion of the filter chamber. In specific instances, the pressure source may comprise a piston pump configured to apply the negative pressure by retracting a piston and to apply the positive pressure by advancing the piston. For example, the pressure source may comprise a syringe configured to apply the negative pressure by retracting a plunger of the syringe and to apply the positive pressure by advancing the plunger of the syringe.

In a fifth aspect, the disclosed technology provides a method for filtering clot from blood. The method comprises pressurizing blood having entrained clot to cause the blood to sequentially flow through (a) a filter chamber wherein clot separates on an upper surface of a filter element and filtered blood substantially free from clot collects in a lower portion of the filter chamber, and (b) a deaeration chamber wherein gas present in said filtered blood separates and collects in an upper portion of the deaeration chamber and passes out through a gas vent.

In some instances, the blood having entrained clot may be pressurized with a piston pump, such as a syringe.

In other instances, the blood may be pressurized with a continuous pump, such as a diaphragm pump, a centrifugal pump, or the like.

In some instances, the methods may further comprise viewing clot which has collected on the upper surface of the filter element though a transparent top of the filter chamber and, optionally, stopping the blood pressurization, removing the transparent top, removing at least a portion of the filter element from the filter chamber, and cleaning clot from the removed at least a portion of the filter element. In such instances, an upper strainer portion of the filter element may be removed while a lower microporous filter component remains in the filter chamber to minimize the risk of clot falling into filtered blood in the lower portion of the filter chamber.

In some instances, the filtered blood may pass from the filter chamber to the deaeration chamber through a one-way valve that prevents backflow from the deaeration chamber to the filter chamber.

In some instances, the filtered blood may pass from the lower portion of the filter chamber to a lower portion of the deaeration chamber. For example, the filtered blood may flow upwardly from the lower portion of the deaeration chamber through a vertical tube and be released into the upper portion of the deaeration chamber allowing the gas to separate from the filtered blood and collect at the top of the deaeration chamber and the filtered blood to collect at the bottom of the deaeration chamber.

In some instances, gas flow through the vent valve may be controlled by a float valve.

In some instances, the filtered blood released from the vertical tube engages a lower surface of a deflector that directs the filtered blood flow downwardly and allows the separated gases to pass upwardly. For example, the float valve may be disposed over an upper surface of the deflector and rise to seal against a vent port when the deaeration chamber fills with blood and falls to open the vent port in response to gas collecting in the upper portion of the deaeration chamber.

In some instances, the blood and clot may be pressurized by aspirating the blood entrained with clot from a patient through an aspiration catheter using a pressure source to apply a negative pressure to the aspiration catheter and using the same pressure source to apply a positive pressure to pressurize the blood and entrained clot to cause the aspirated blood entrained with clot to flow into the filter chamber. For example, the pressure source may comprise a piston pump and applying the negative pressure may comprise retracting a piston of the piston pump and applying the positive pressure may comprise advancing the piston of the piston pump.

In some instances, pressure source may comprise a syringe and applying the negative pressure may comprise retracting a plunger of the syringe and applying the positive pressure comprises advancing the plunger of the syringe.

In some instances, the methods may further comprise returning filtered blood from the deaeration chamber to the patient. For example, the filtered blood may be returned to the patient through an access sheath used to introduce the aspiration catheter.

In a sixth aspect, the disclosed technology provides a system for use with an aspiration catheter and a blood return cannula. The system comprises a filter chamber, a first pressure source and a second pressure source. The filter chamber has an inlet, an outlet, and a filter element therein that divides the chamber into a clot collecting portion and a blood transfer portion. The first pressure source is configured to connect to a proximal end of the aspiration catheter and to the inlet of the filter chamber and to generate a negative pressure to draw clot and blood from the aspiration catheter and to generate a positive pressure to transfer the drawn blood and clot to the inlet of the filter chamber. The second pressure source is configured to fill with blood from the blood transfer portion of the filter chamber as the first pressure source generates positive pressure and to generate a positive pressure to deliver the filtered to the blood return cannula.

In some instances, the amount of clot collected in the clot collecting portion is externally visible.

In some instances, the filter is removable from the filter chamber to allow clot to be removed from the filter element when the chamber is not pressurized.

In some instances, the first pressure source comprises a syringe.

In some instances, the second pressure source comprises a syringe.

In some instances, the filter chamber has a vertical dimension, and the filter element is oriented horizontally.

In a seventh aspect, the disclosed technology provides a method for clot aspiration and blood return. The method comprises aspirating blood and clot from the vasculature of a patient into an inlet of a filter chamber having a filter element therein where the clot collects on a surface of the filter and the blood passes to a receptacle, The filtered blood in the receptacle may be separately pressurized to return the filtered blood to the patient.

In some instances, aspirating the blood and clot may comprise applying a negative pressure from a first pressure source to an aspiration catheter in the patient's vasculature and thereafter applying a positive pressure from the first pressure source to transfer the blood and clot to the filter chamber. For example, the first pressure source may comprise a first syringe and applying the negative pressure may comprise retracing a plunger of the syringe and applying the positive pressure may comprise advancing the plunger of the syringe.

In some instances, separately pressurizing the receptacle to return the filtered blood to the patient may comprise applying a positive pressure from a second pressure source to the receptacle. For example, the second pressure source may comprise a second syringe and applying the positive pressure may comprise advancing a plunger of the second syringe.

In an eighth aspect, the disclosed technology provides a system for use with an aspiration catheter and a blood return cannula. The system comprises a filter chamber and at least a first pressure source. The filter chamber includes an inlet, an outlet, and a filter element therein that divides the chamber into a clot collecting portion and a blood transfer portion. The at least a first pressure source is connectable to a proximal end of the aspiration catheter and to the inlet of the filter chamber, and the first pressure source is configured to generate a negative pressure to draw clot and blood from the aspiration catheter and to generate a positive pressure to transfer the drawn blood and clot to the inlet of the filter chamber. The blood transfer portion of the filter chamber is configured to return filtered blood to the blood return cannula; and the amount of clot collected in the clot collecting portion is externally visible and the filter is removable from the filter chamber to allow clot to be removed from the filter element when the chamber is not pressurized.

In some instances, the systems may further comprise a second pressure source configured to fill with blood from the blood transfer portion of the filter chamber as the first pressure source generates positive pressure, wherein the second pressure source is further configured to generate a positive pressure to deliver the filtered to the blood return cannula.

In some instances, the first pressure source may comprise a syringe.

In some instances, second pressure source may comprise a syringe.

In some instances, the filter chamber may have a vertical dimension and the filter element may be oriented horizontally, and a top of the filter chamber may be removable to allow the filter element to be lifted to remove accumulated clot while leaving the filtered blood in the blood transfer portion.

In a ninth aspect, the disclosed technology provides a method for clot aspiration and blood return. The method comprises aspirating blood and clot from the vasculature of a patient into an inlet of a filter chamber having a filter element therein, wherein the clot collects on a surface of the filter and the blood passes to a receptacle. The filter may be removed from the chamber and cleaned the removed filter when excess clot has collected on the filter surface.

In some instances, aspirating the blood and clot comprises applying a negative pressure from a first pressure source to an aspiration catheter in the patient's vasculature and thereafter applying a positive pressure from the first pressure source to transfer the blood and clot to the filter chamber. For example, the first pressure source may comprise a first syringe and applying the negative pressure may comprise retracting a plunger of the syringe and applying the positive pressure may comprise advancing the plunger of the syringe.

In some instances, separately pressurizing the receptacle to return the filtered blood to the patient may comprise applying a positive pressure from a second pressure source to the receptacle.

In a tenth aspect, the present technology provides method for clot aspiration which incorporate pressure sensing within a blood and clot aspiration pump assembly. The methods comprise translating a positive displacement element in a chamber to draw blood and clot through a lumen of an aspiration catheter having a distal opening located in a patient's vasculature into a receiving volume of the chamber. Pressure within the receiving volume of the chamber is monitored as the displacement element is being translated, and a rate or pattern of translating the positive displacement element is controlled to maintain a pressure in the receiving volume at a target value or within a target range.

In some instances, translating the positive displacement element in the chamber to draw blood and clot through a lumen of an aspiration catheter may comprise powered retraction of a plunger in a chamber comprising a syringe barrel, including, for example, fluidically or electrically powered retraction.

In some instances, monitoring pressure within the receiving volume of the chamber may comprise directly measuring the pressure with a pressure sensor located within the receiving volume.

In other instances, monitoring pressure within the receiving volume of the chamber may comprise indirectly measuring the pressure with a pressure or force sensor located externally of the receiving volume.

In some instances, controlling the rate of translating the positive displacement element may comprise maintaining a target pressure in the receiving volume in a range from −760 mmHg to −100 mmHg.

In some instances, the target pressure may be maintained above a vacuum level that would cause hemolysis.

In some instances, the vacuum level may be in a range from −760 mmHg to −100 mmHg.

In some instances, controlling the rate of translating the positive displacement element may comprise retracting the positive displacement element in a series of steps to cause a pressure hammer effect.

In an eleventh aspect, the present technology provides a system for use with an aspiration catheter where the system comprises a chamber and a positive displacement element translatably mounted in the chamber to draw blood and clot through a lumen of the aspiration catheter into a receiving volume of the chamber. A sensor is configured to measure pressure within the receiving volume of the chamber as the displacement element is being translated, and a controller is configured to receive an output of the sensor and to control a rate of translating positive displacement element to maintain a target pressure in the receiving volume at a target value or within a target range.

In some instances, the positive displacement element and the chamber may comprise a plunger in a syringe assembly.

In some instances, the systems may further comprise a powered driver coupled to the positive displacement element and controlled by the controller, for example being a fluidically powered driver or an electrically powered driver.

In some instances, the sensor may comprise a pressure sensor disposed within the receiving volume and configured to measure the pressure directly.

In other instances, the sensor may comprise a pressure or force sensor disposed externally of the receiving volume and configured to measure the pressure indirectly.

In some instances, the controller may be configured to control translation of the positive displacement element comprises at a rate selected to maintain a target pressure in the receiving volume in a range from −760 mmHg to −100 mmHg.

In some instances, the target pressure is maintained above a vacuum level that would cause hemolysis.

In some instances, the vacuum level may be in a range from −740 mmHg to −300 mmHg.

In some instances, controlling the rate of translating the positive displacement element may comprise retracting the positive displacement element in a series of steps to cause a pressure hammer effect.

In a tenth aspect, the present technology provides an aspiration device for use with (a) an aspiration catheter and (b) a fluidic actuator configured to deliver a pressurized drive fluid, said aspiration device comprises an aspirator including an aspirator displacement element, an aspirator cylinder, and an aspirator port. The aspirator port is configured to connect to an aspiration lumen of the aspiration catheter. A fluidic driver includes a driver displacement element, and a coupling element is configured to drive the aspirator displacement element in tandem in with the driver displacement element to draw portions of blood and clot through the aspirator port and into and from the aspiration cylinder when a distal port of the aspiration catheter is in a patient blood vessel proximate clot. A travel distance of the coupling element is adjustable to control the volume of blood and clot portions aspirated into the aspiration catheter.

In some instances, the aspiration device of claim 250, further comprising travel stops that limit the travel of the coupling elements. For example, the travel stops may comprise pins and ledges controlled by a knob.

In an eleventh aspect, the disclosed technology provides an alternative aspiration device for use with an aspiration catheter. The alternative aspiration device comprises a chamber having a pressure port, a blood inlet port, and a blood outlet port. A pump has a positive pressure port and a negative pressure port and is connected to the chamber by a valve. The valve is configured to selectively connect the positive and negative pressure ports of the pump to the pressure port of the chamber, and a controller is configured to control the valve to selectively apply negative and positive pressure from the pump to an interior of the chamber to draw blood into the chamber interior through the blood inlet port and to deliver blood from the interior through the blood outlet port.

In some instances, the pressure port is located on an upper region of the camber and the blood inlet and blood outlet ports are located on a lower region of the chamber.

In some instances, the blood inlet and blood outlet ports each comprise a one-way flow element to control fluid flow direction.

In some instances, the chamber pressure port comprises a float valve to prevent blood from being extracted by the pump.

In a twelfth aspect, the disclosed technology provides an aspiration system comprising an aspiration catheter and an aspirator. The aspirator is configured to connect to a proximal end of the aspiration catheter and to generate a negative pressure to aspirate blood and clot into an aspiration lumen of the aspiration catheter. A first pressure sensor is coupled to a proximal end of the aspiration catheter, and a second pressure sensor is coupled to an inlet of the aspirator. Control circuitry is configured to receive pressure measurements from the first and second pressure sensors and to detect clogging based upon the pressure measurements.

In some instances, a pressure detected by the first pressure sensor lower than expected indicates a blockage in the aspiration catheter.

In some instances, a pressure detected by the second pressure senor lower than expected indicates a blockage in a line connecting the aspiration catheter to the aspirator.

For example, described herein are methods of closed-loop clot removal and blood return that are configured to withdrawal blood from a body at a rate that does not depend on the rate that blood is returned to the body. For example any of these methods may include: applying aspiration through a first portion of a blood removal and return circuit to draw a clot material and blood into the blood removal and return circuit at a first flow rate; and applying positive pressure through the blood removal and return circuit to drive the clot material and blood into a clot collection chamber within a second portion of the blood removal and return circuit, wherein the clot collection chamber comprises a capacitive air reservoir that is configured to hold a minimum volume of air between a filter and a visualization window of the clot collection chamber; filtering the blood within the clot collection chamber and passing the filtered blood into a second chamber; and returning blood from the second chamber to the patient at a second flow rate.

Any of these methods may include compressing or expanding the capacitive air reservoir as blood passes through the clot collection chamber. In some cases, the method may include allowing flow from the clot collection chamber to the second chamber but not from the second chamber to the clot collection chamber, e.g., using one or more one-way valves.

Any of these methods may include removing the visualization window of the clot collection chamber to remove clot material from the clot collection chamber. For example, removing the visualization window may comprise removing the visualization window without breaking the blood removal and return circuit. The clot collection chamber may be isolated from the upstream and downstream components of the blood removal and return circuit by one or more valves, and in particular, one-way valves.

In some cases the minimum volume (e.g., of the capacitive air reservoir(s)) may be 10 cc or less (e.g., between 10 cc-5 cc, etc.).

The second flow rate may be less than or equal to the first flow rate. For example the first flow rate may be between 6 cc/sec and 400 cc/sec (e.g., between about 6 cc/sec-300 cc/sec, between about 6 cc/sec and 250 cc/sec, between about 6 cc/sec and 200 cc/sec, between about 6 cc/sec and 150 cc/sec, between about 6 cc/sec and 100 cc/sec, between about 6 cc/sec and 80 cc/sec, between about 6 cc/sec and 60 cc/sec, between about 6 cc/sec and 40 cc/sec, etc.). The second flow rate may be between about 6 cc/sec and 20 cc/sec, between about 6 cc/sec and 18 cc/sec, between about 6 cc/sec and 16 cc/sec, between about 6 cc/sec and 14 cc/sec, between about 6 cc/sec and 12 cc/sec, between about 6 cc/sec, etc.).

The second chamber may have a second capacitive air reservoir that is configured to vent air from the second chamber. In some examples the second chamber comprises a de-airing chamber.

In any of these examples, the clot collection chamber may be sealed to prevent air from outside of the clot collection chamber from entering the clot collection chamber. The clot collection chamber (e.g., the window) may be attached by a threaded member that may be opened/closed to allow the window portion to be opened/removed, the clot material to be removed from the filter, and the window portion to be replaced.

In any of these apparatuses, the clot collection chamber may include a wiper within the clot collection chamber configured to wipe the visualization window. The wiper may be actuated from outside of the chamber.

For example, a closed-loop clot removal and blood return system may include: an aspiration line configured to fluidically couple to an aspiration catheter to remove clot and blood from a patient; a pressure source configured to apply aspiration through the aspiration line; a positive pressure lumen in fluid communication with the aspiration line; a clot collection chamber coupled to the positive pressure lumen and configured to receive the clot and blood from the patient, wherein the clot collection chamber comprises a viewing window and a capacitive air reservoir between with viewing window and a filter that is configured to filter the clot material from the blood, an inlet above the filter, and an outlet below the filter, wherein the capacitive air reservoir is configured to hold a minimum volume of air between the viewing window and the filter; a second chamber having a second inlet that is fluidically coupled to the outlet of the clot collection chamber and a second outlet that is lower than the second inlet; a one-way valve between the outlet and the second inlet, configured to allow flow from the clot collection chamber to the second chamber but not from the second chamber to the clot collection chamber; and a blood return line fluidically coupled to the second outlet. The capacitive air reservoir may be configured to compress or expand as blood passes through the clot collection chamber.

In any of these apparatuses, the visualization window of the clot collection chamber may be removable to allow clot material to be removed from the clot collection chamber. The minimum volume of the capacitive air reservoir may be 10 cc or less (e.g., between about 2 cc and 50 cc, between about 5 cc and 40 cc, between about 5 cc and 25 cc, between about 5 cc and 20 cc, between about 5 cc and 15 cc, etc.). The size of the capacitive air reservoir may be determined as the space above the inlet, which may correspond to the visualization window. In some cases the second chamber has a second capacitive air reservoir that is configured to vent air from the second chamber. The second chamber may have a capacitive air reservoir that is the same size as, or larger than, the first capacitive air reservoir. In some cases the second chamber comprises a de-airing chamber.

The clot collection chamber may be sealed to prevent air from outside of the clot collection chamber from entering the clot collection chamber. The clot collection chamber may include a wiper within the clot collection chamber configured to wipe the visualization window. The pressure source may be configured to apply aspiration to the aspiration line at a first rate and to apply positive pressure to the positive pressure lumen at a second rate that is different from the first rate.

Examples of systems, methods and apparatuses that may benefit from the features described herein may be described in one or more of the following patents and publications: U.S. Pat. Nos. 6,059,745; 11,096,712; 11,400,255; 11,464,528; US2015/017782; US2015/0327875; US2017/0181760; US2017/0290598; US2019/0108540; US2019/0239910; US2022/0280171; US2022/0338887; and US2022/0379084.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically illustrates one example of an aspiration apparatus as described herein.

FIGS. 2A-2E schematically illustrate examples of fluidic drives that may be used with an aspiration apparatus as described herein.

FIG. 3 schematically illustrates one example of a pneumatically powered system.

FIG. 4 shows an example of a pneumatic internal operation diagram for control of syringe plunger.

FIG. 5 illustrates and describes details of controlling plunger position using pneumatics and a piston.

FIG. 6A shows an example of control of plunger position using pneumatics and a piston.

FIG. 6B illustrates a powered syringe with a volume selector in accordance with the disclosed technology.

FIGS. 6C and 6D illustrate the powered syringe of FIG. 6A set for 15 CC retraction where a retraction pin of the volume selector knob hits the closest ledge on the carriage.

FIGS. 6E and 6F illustrate the powered syringe of FIG. 6A set for 30 CC retraction where a retraction pin of the volume selector knob hits a middle ledge on the carriage.

FIGS. 6G and 6H illustrate the powered syringe of FIG. 6A set for 60 CC retraction where a retraction pin of the volume selector knob hits a final ledge on the carriage.

FIG. 61 illustrates a direct coupled aspiration syringe pneumatic system in accordance with the disclosed technology.

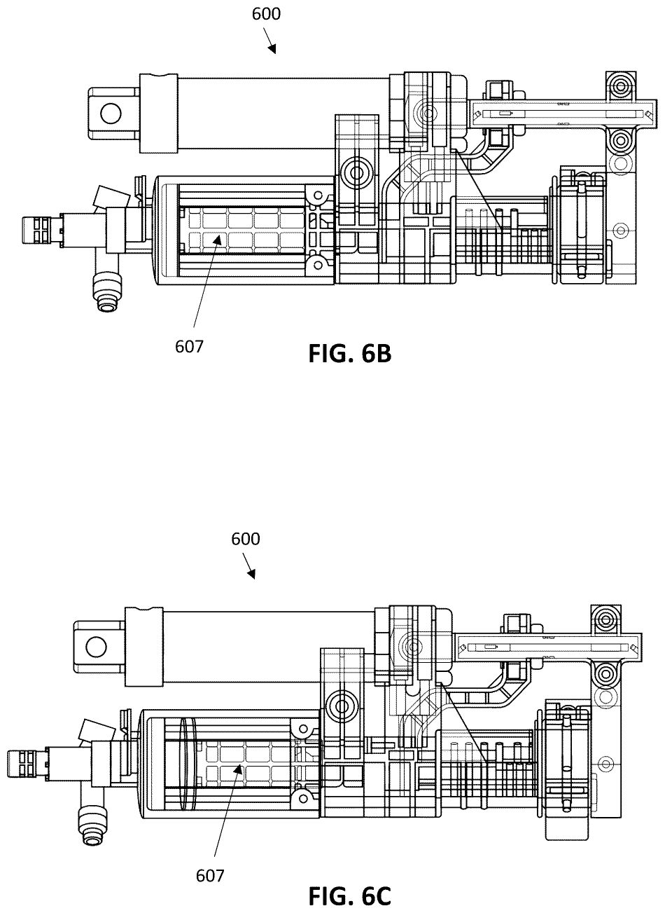

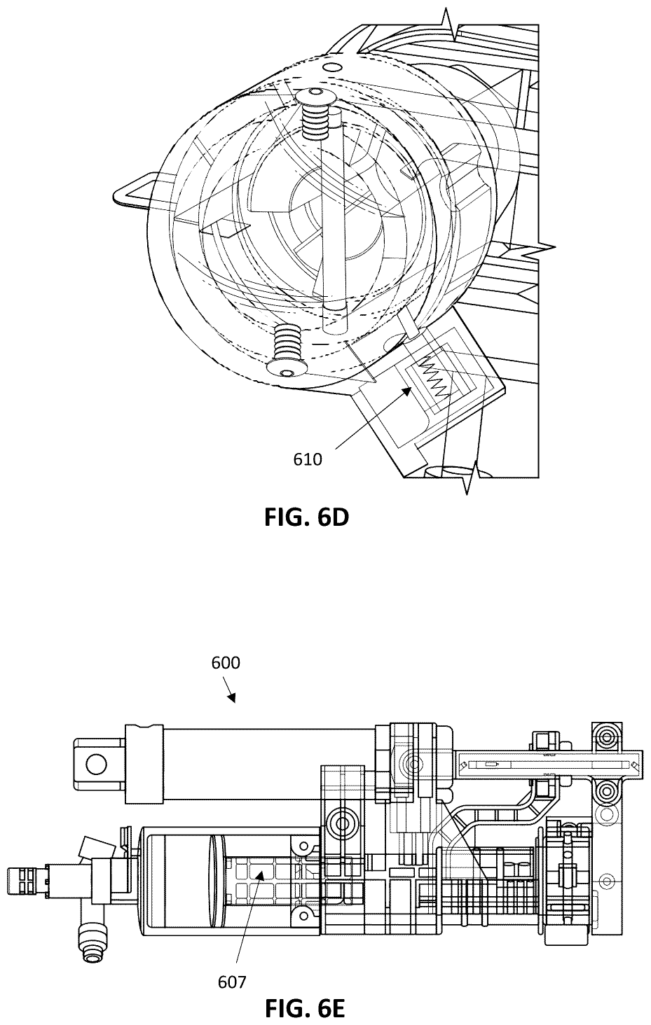

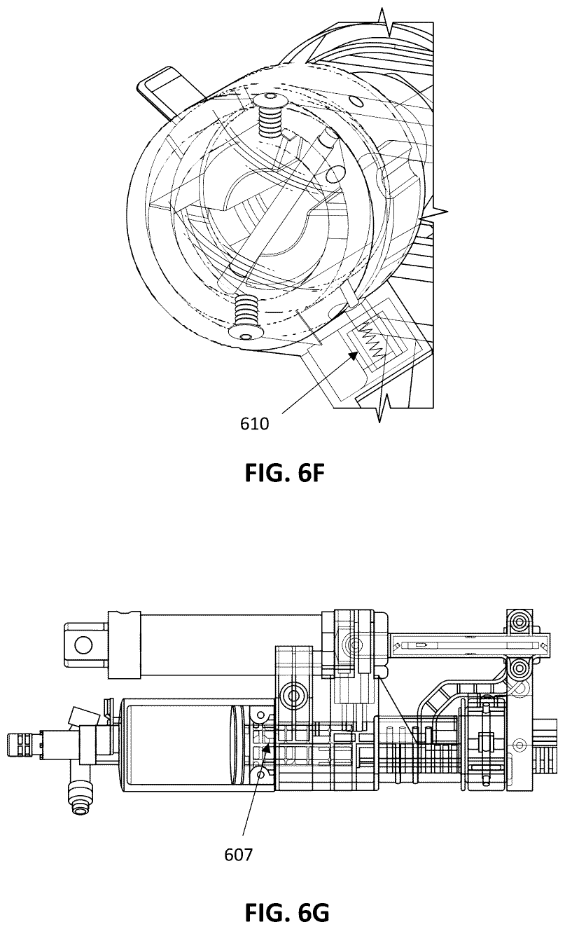

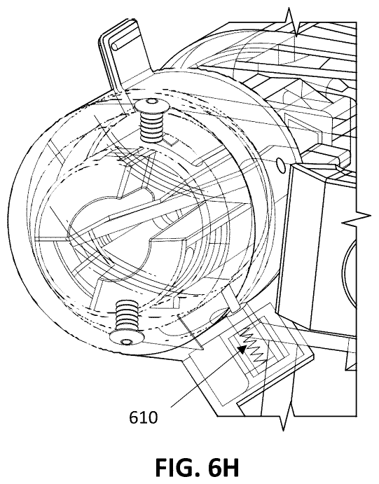

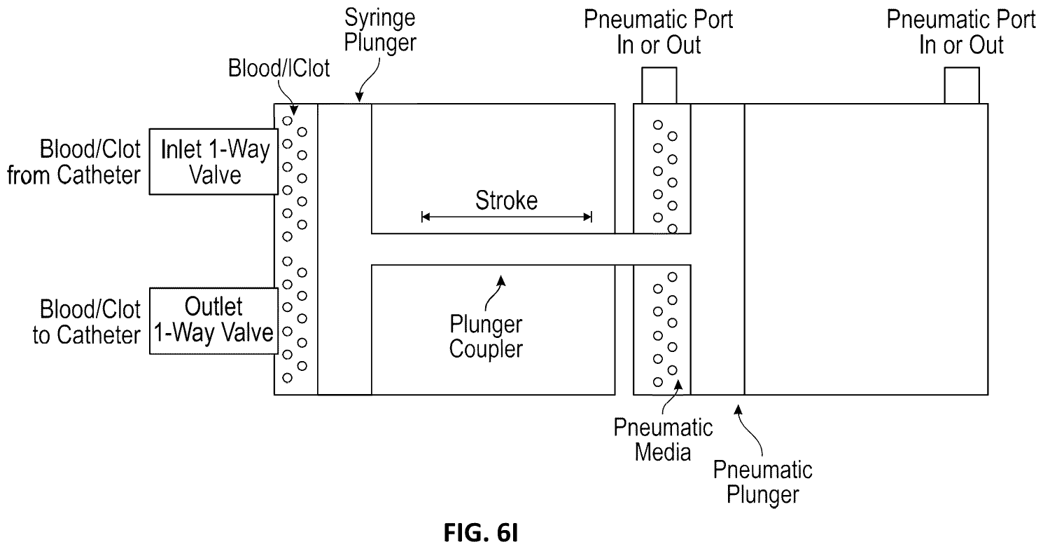

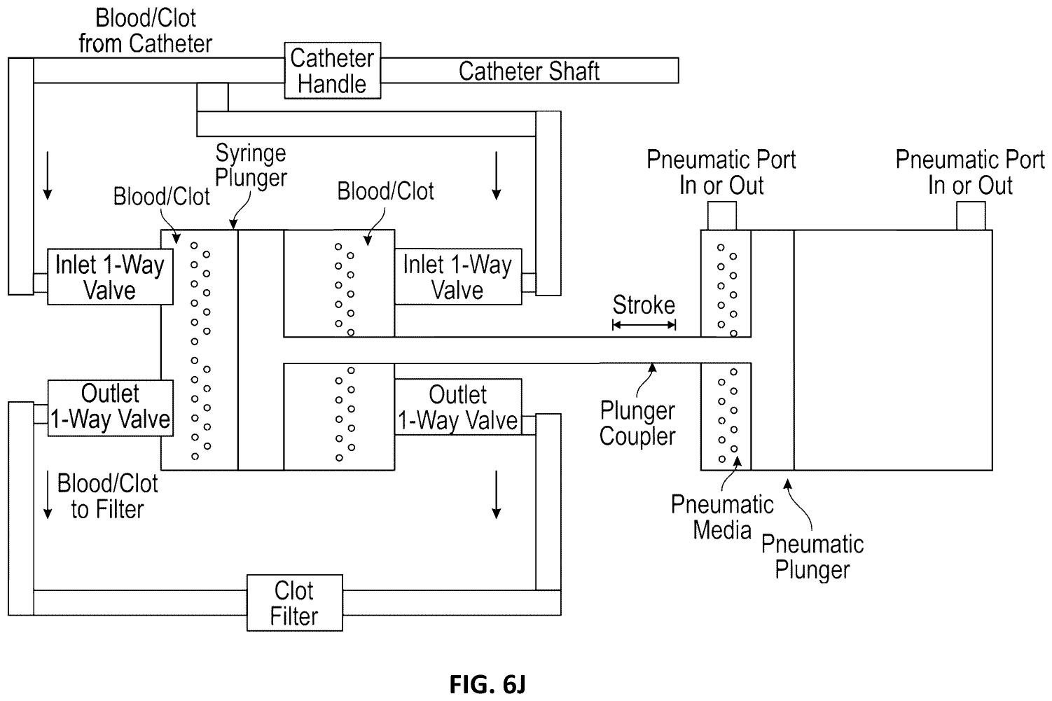

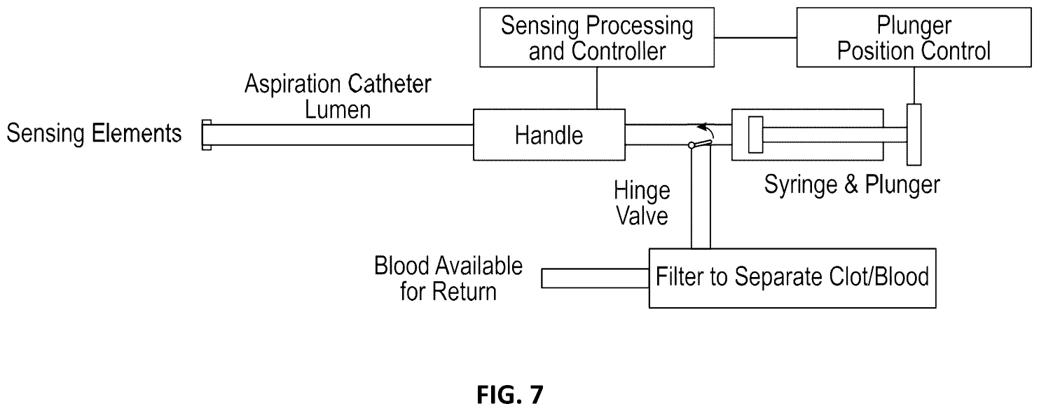

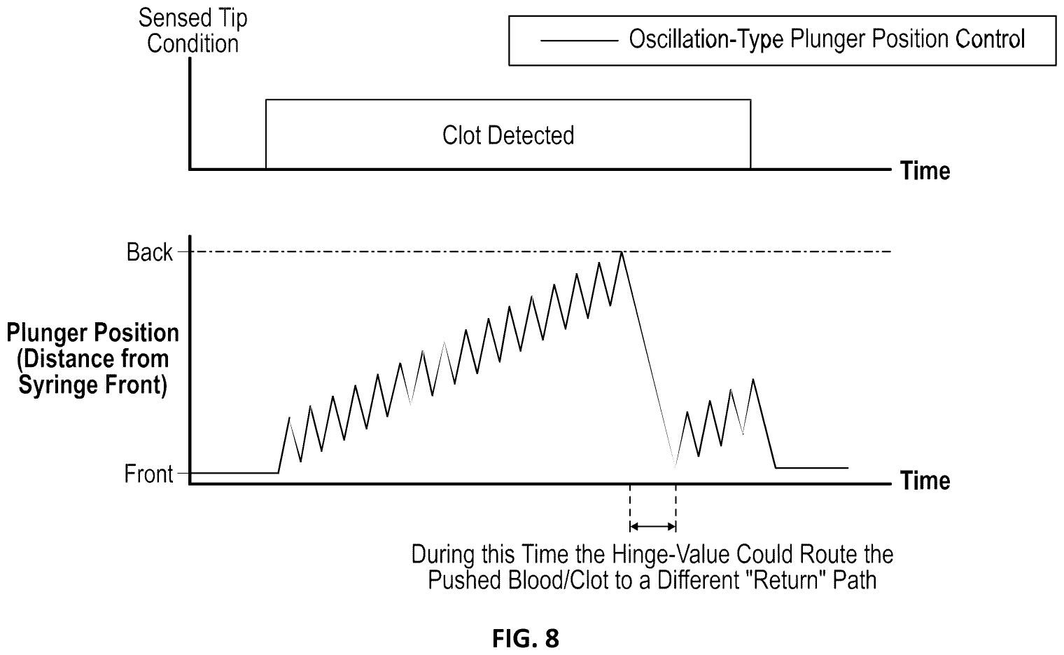

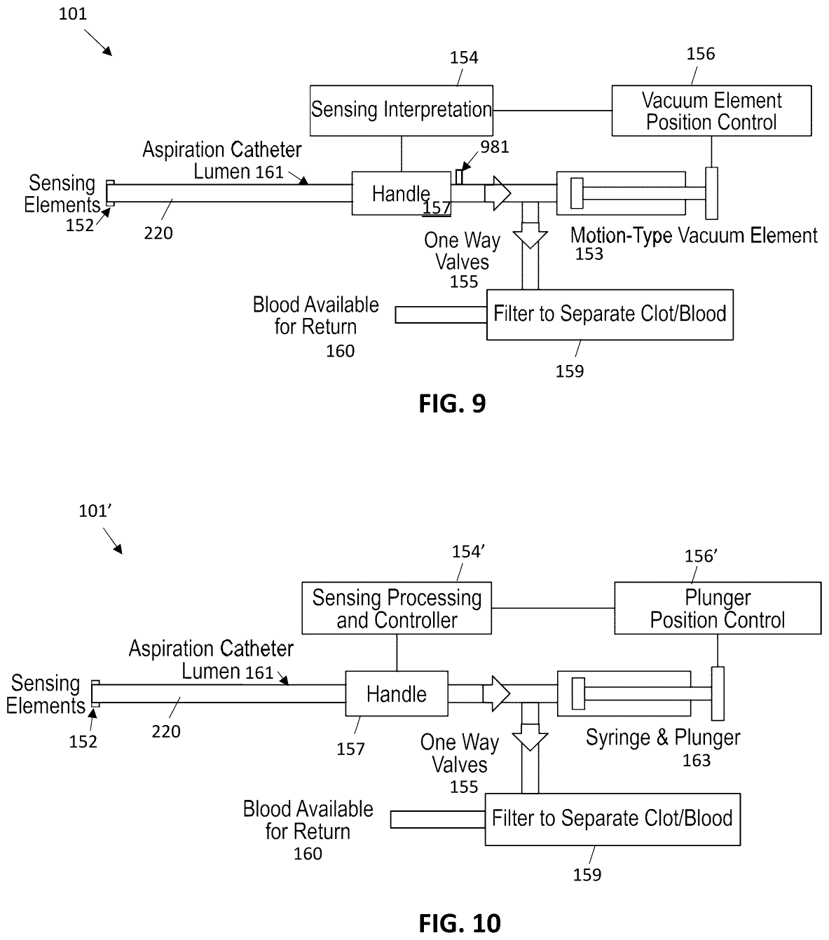

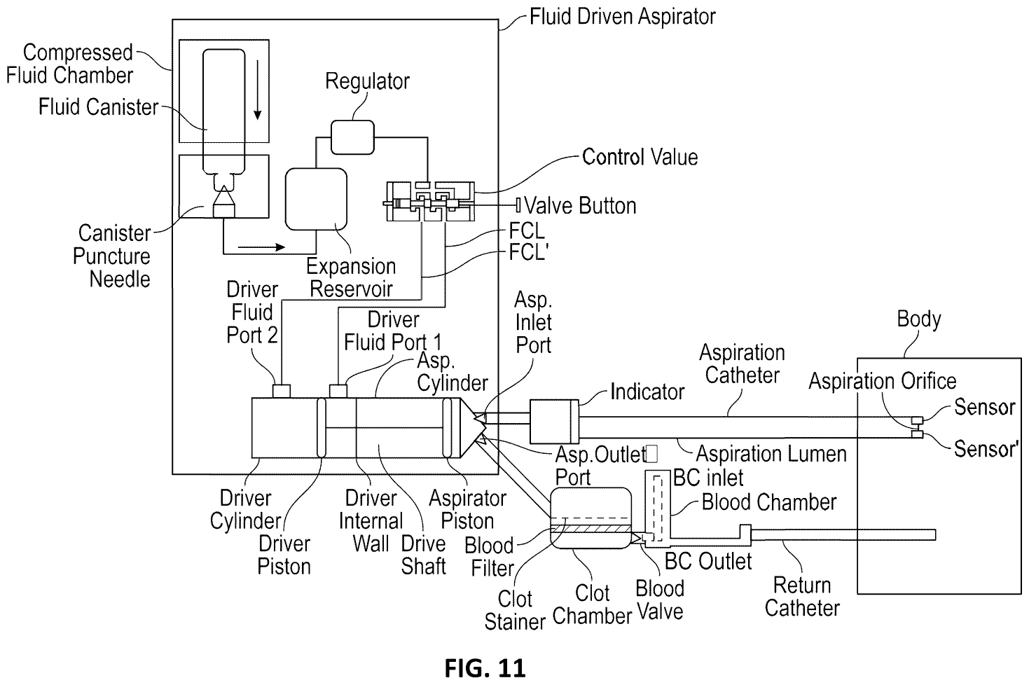

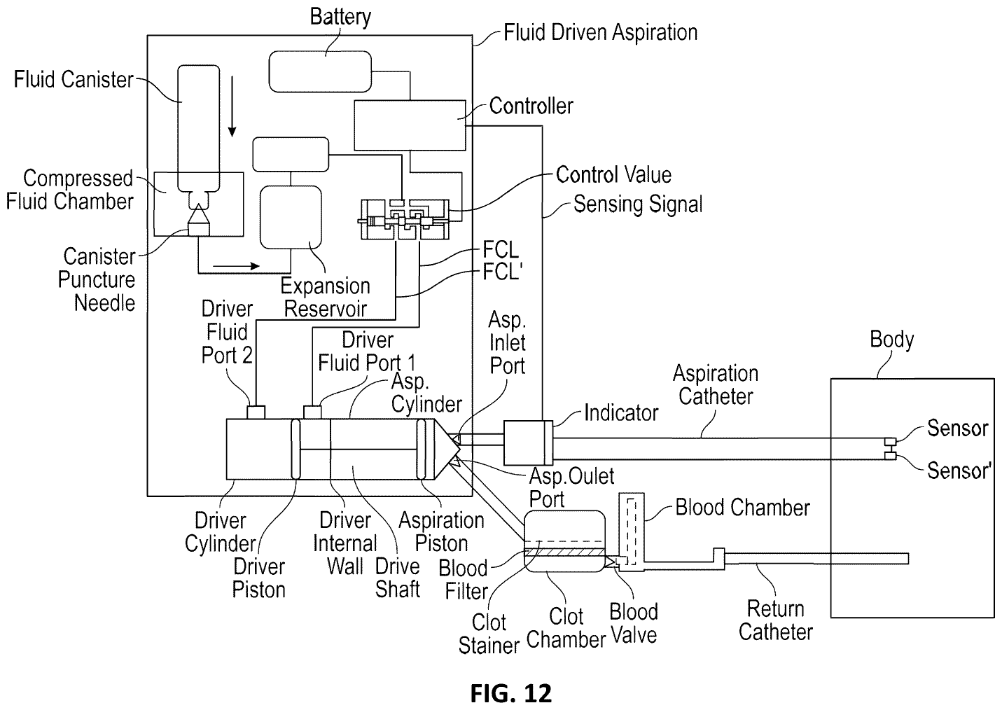

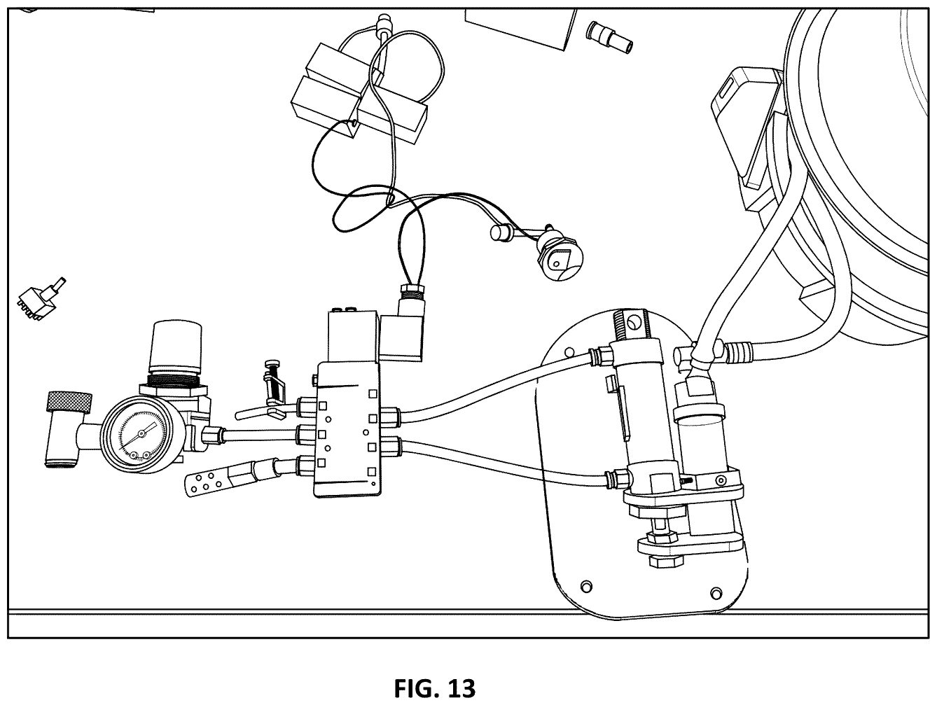

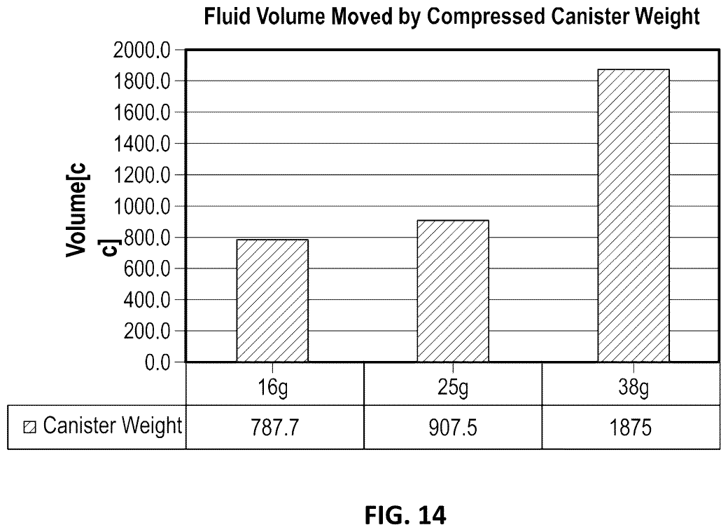

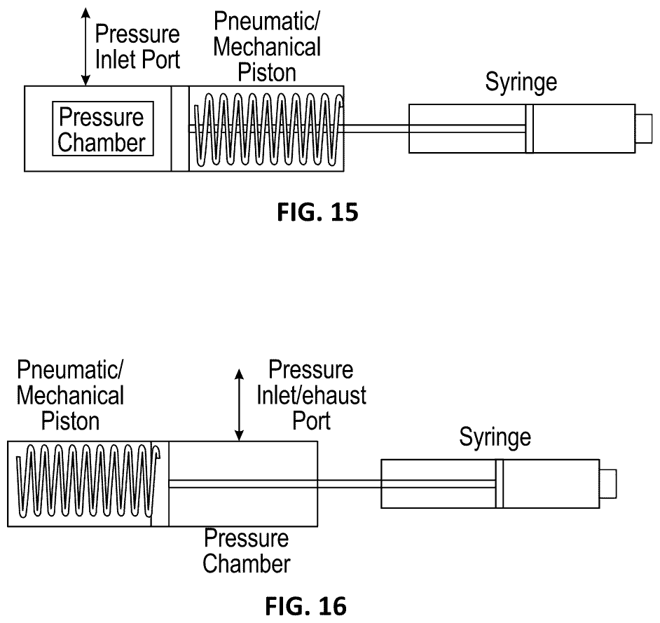

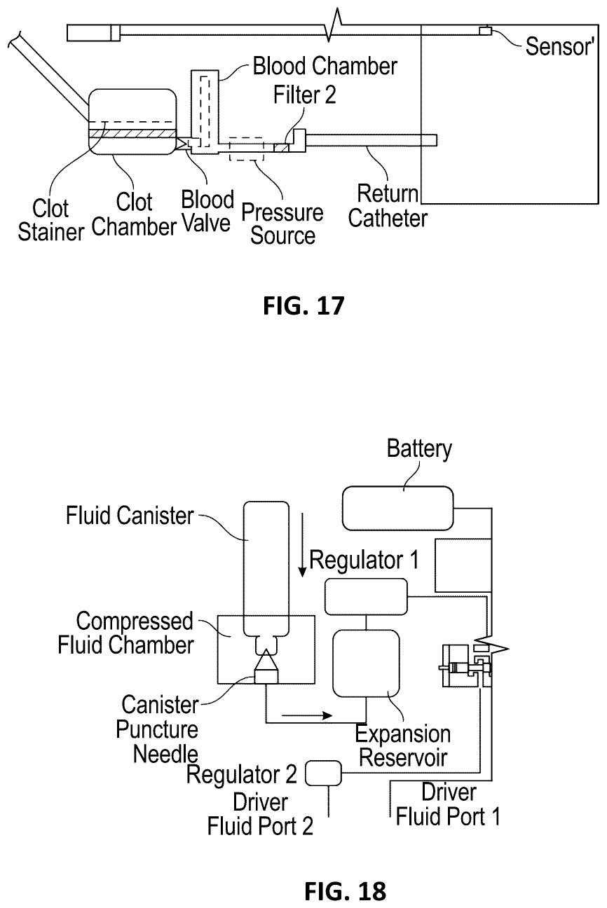

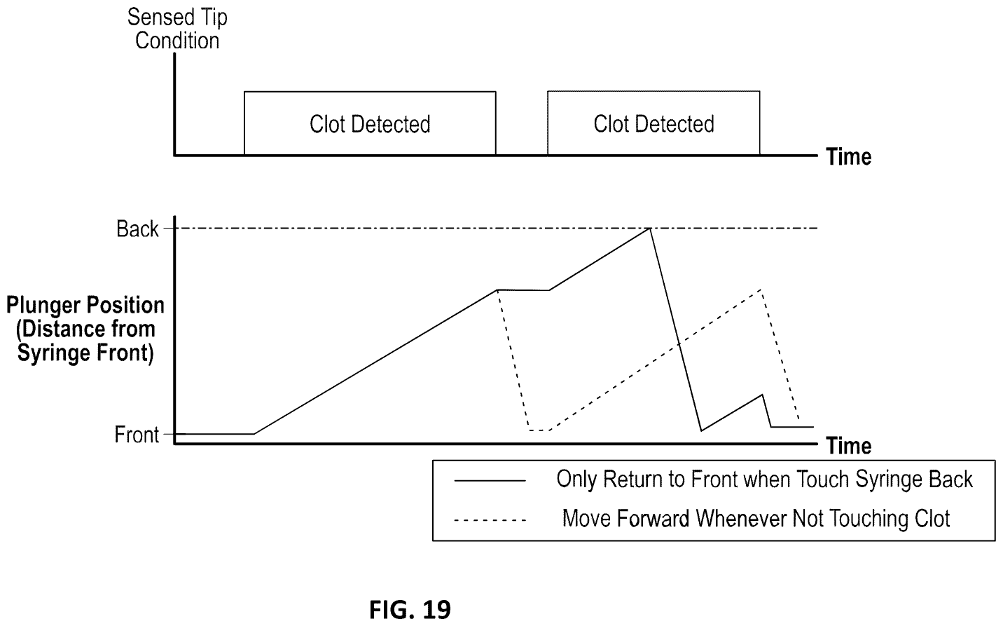

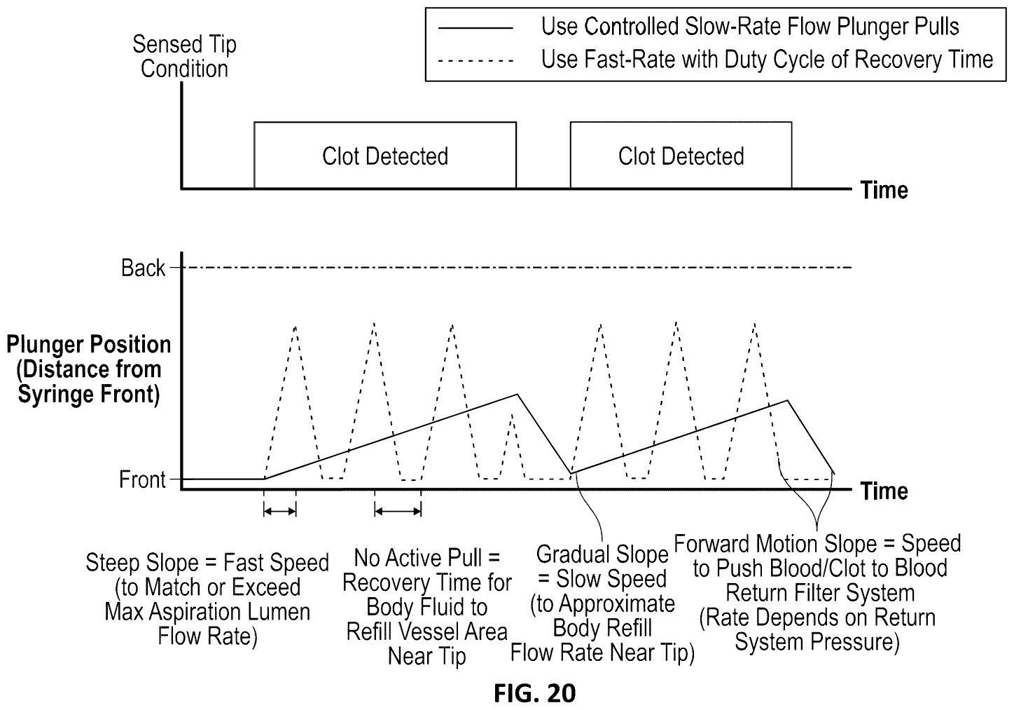

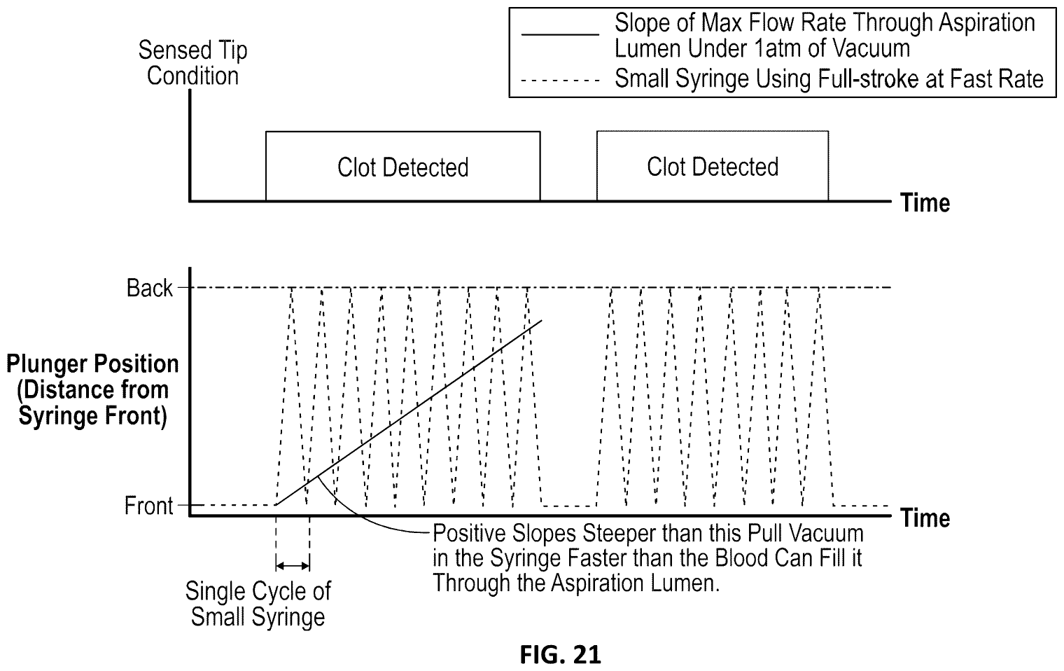

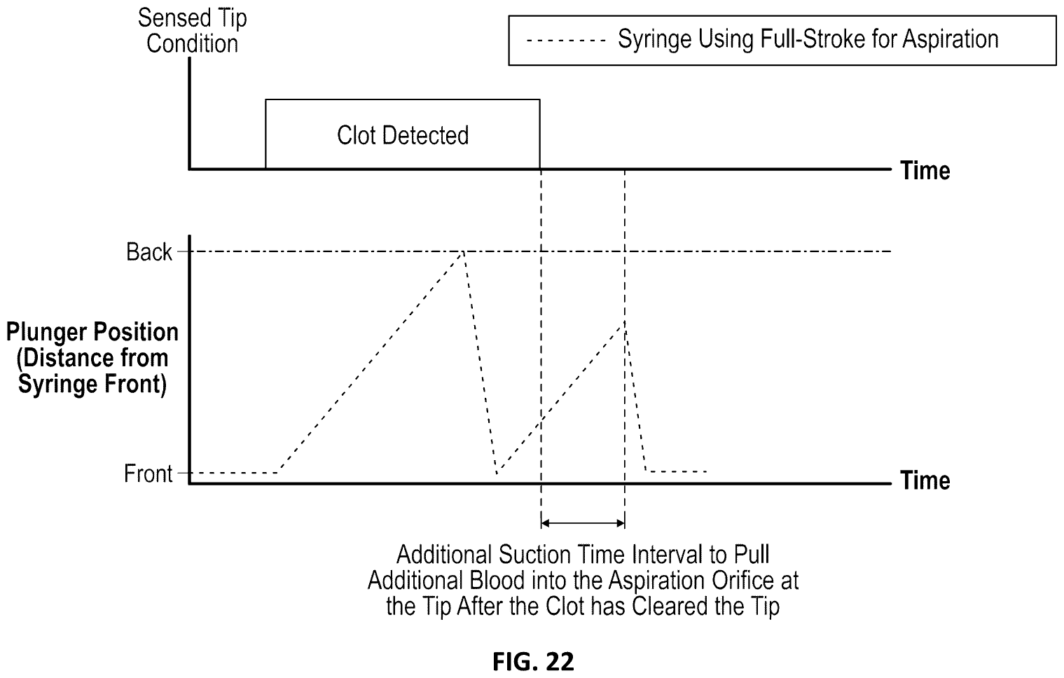

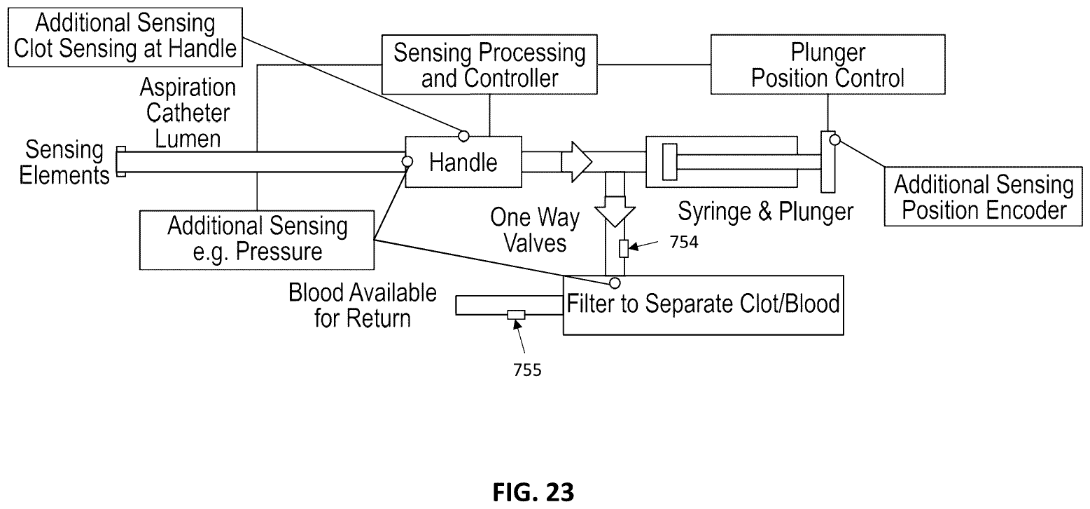

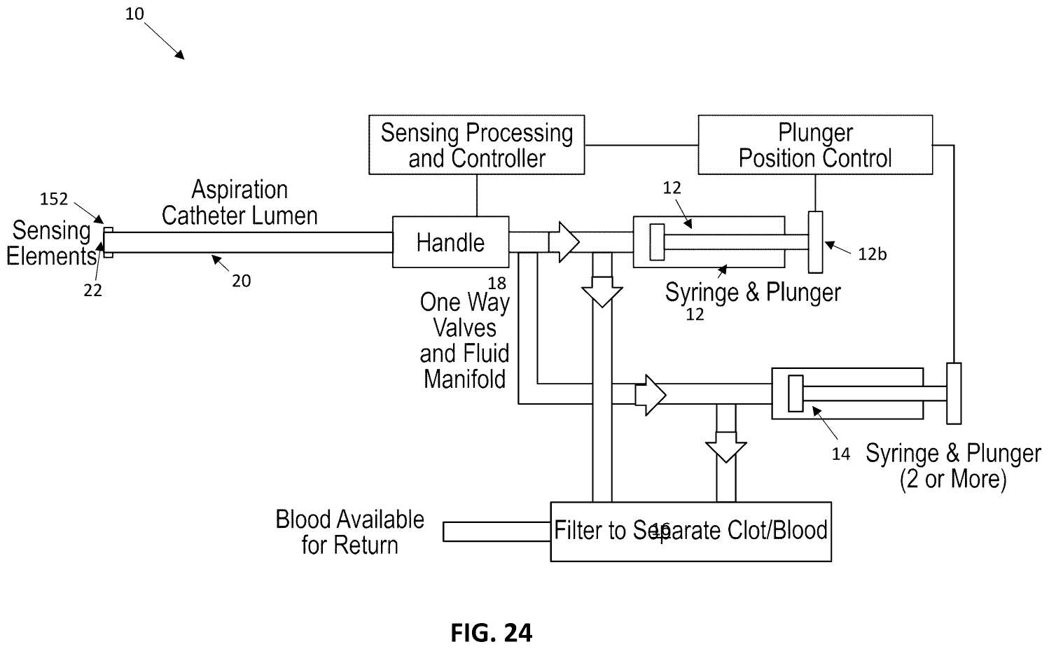

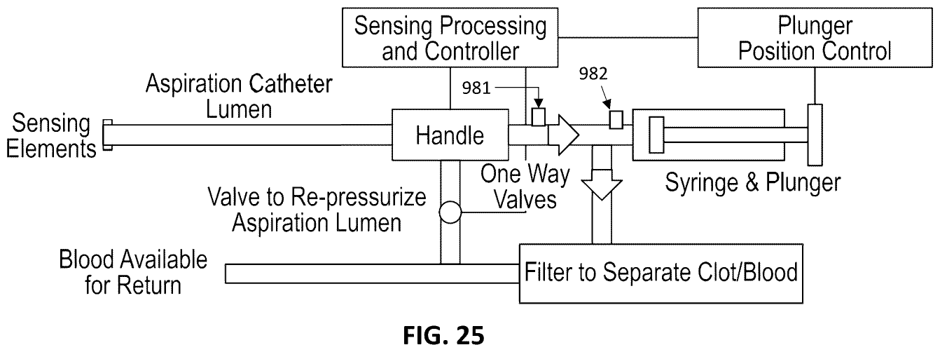

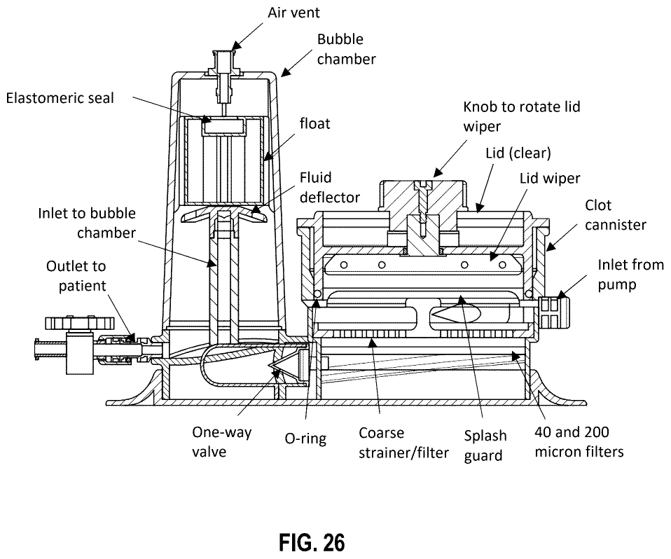

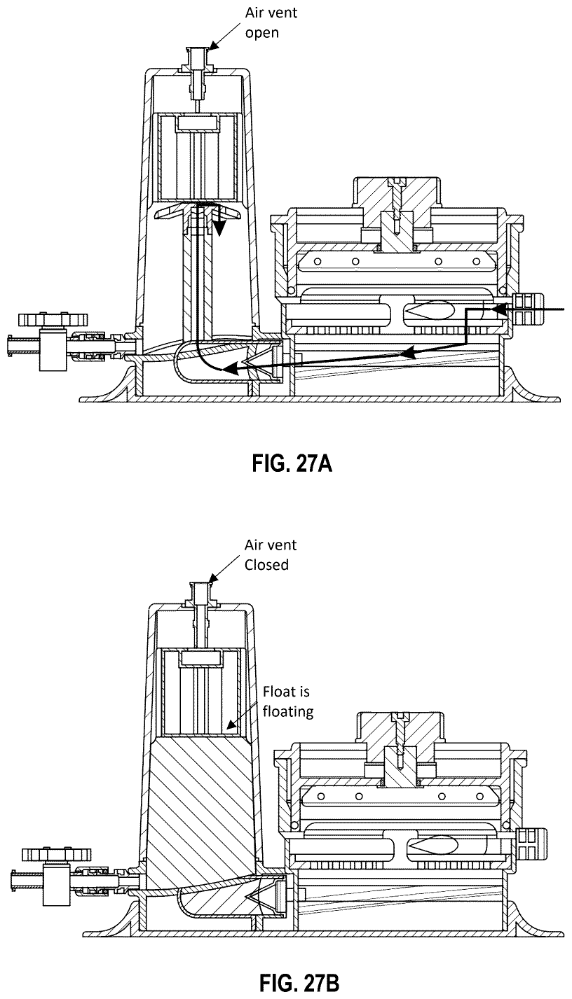

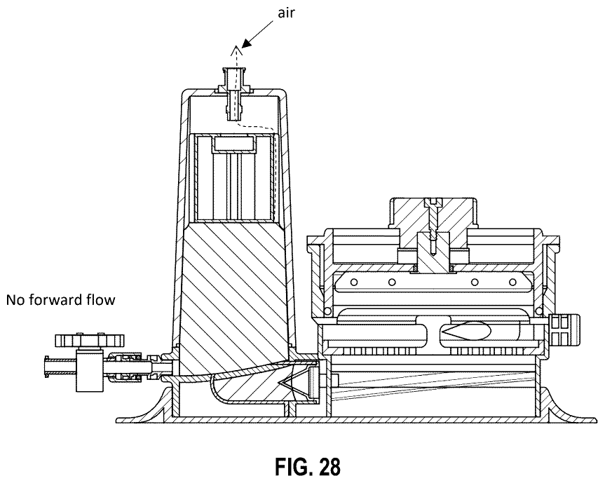

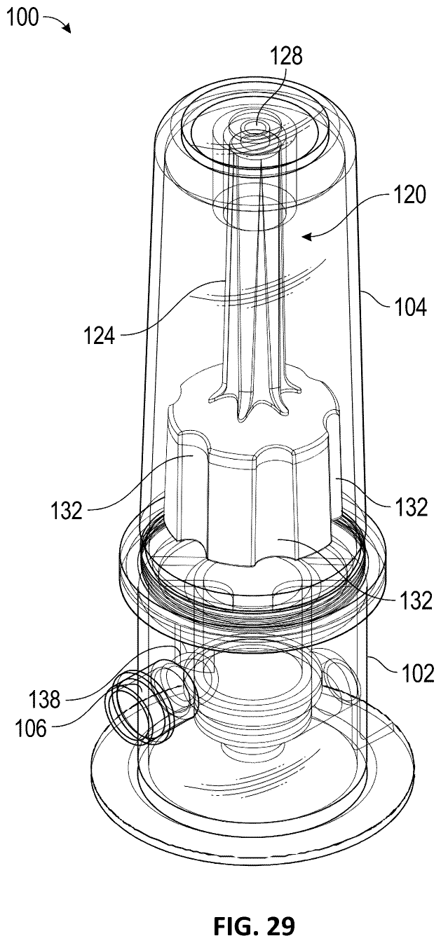

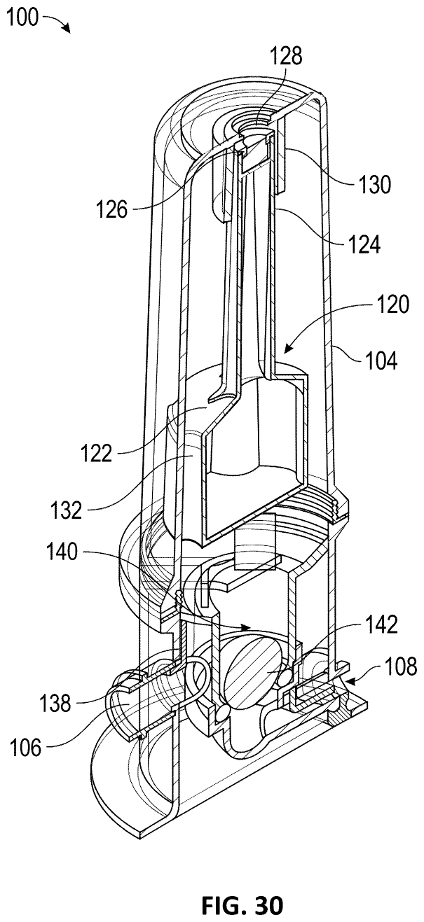

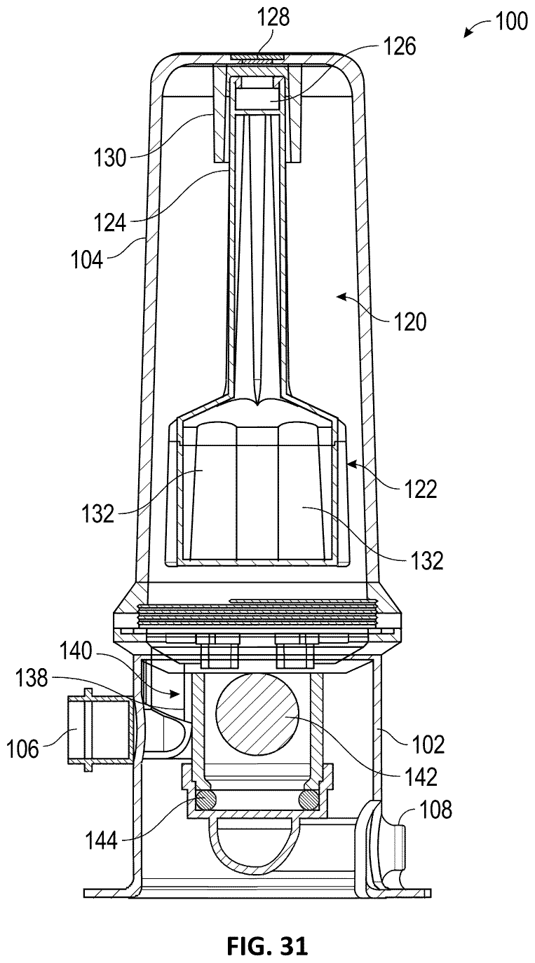

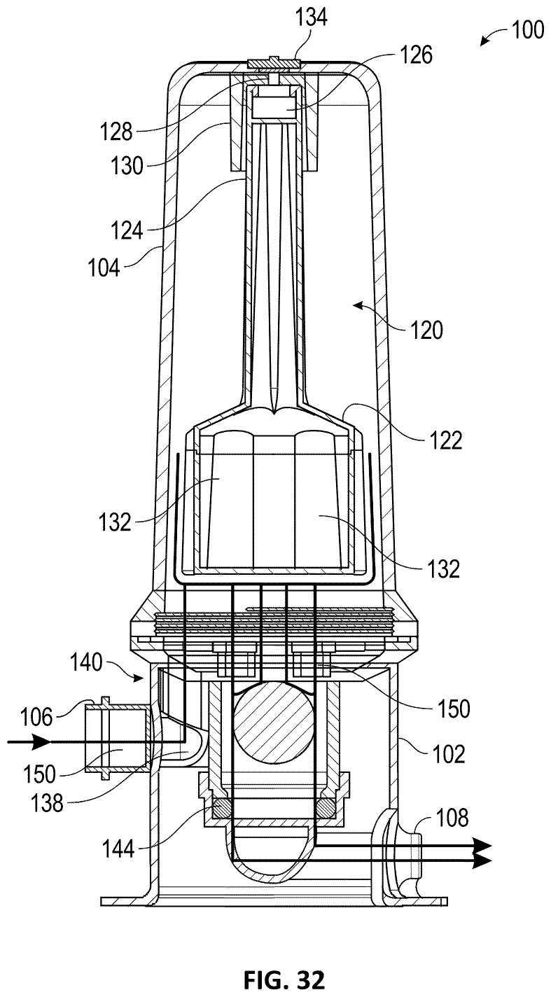

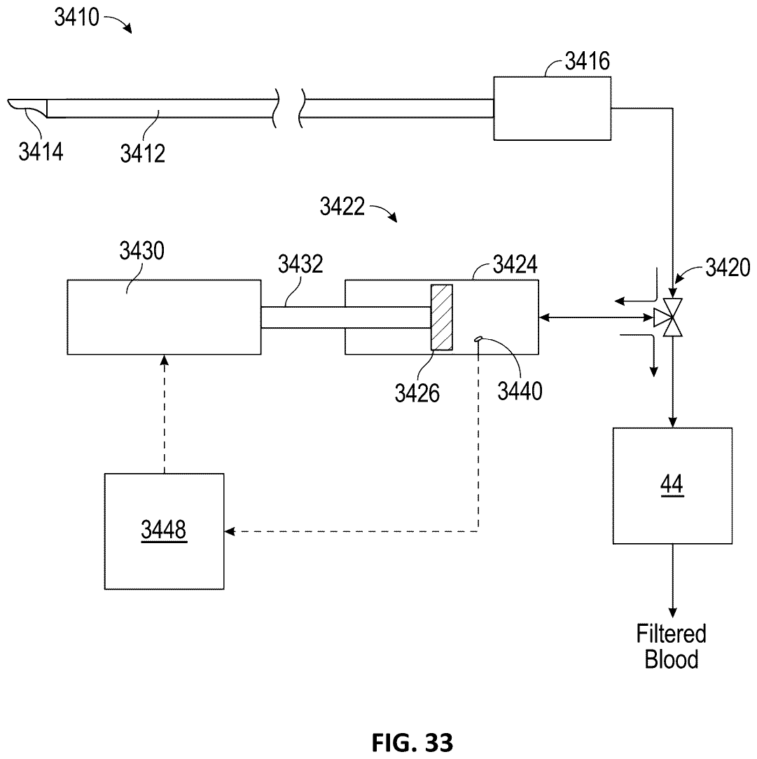

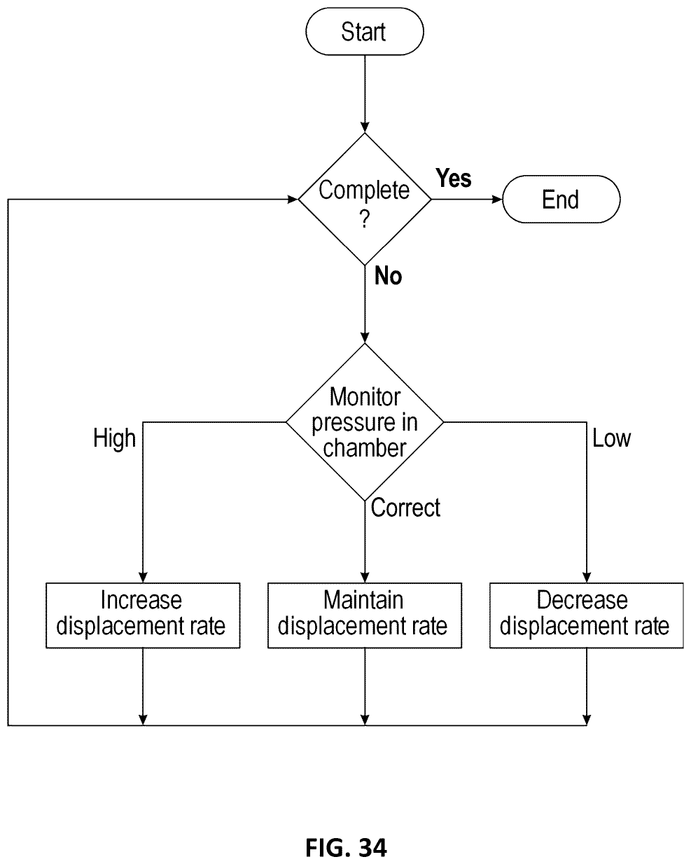

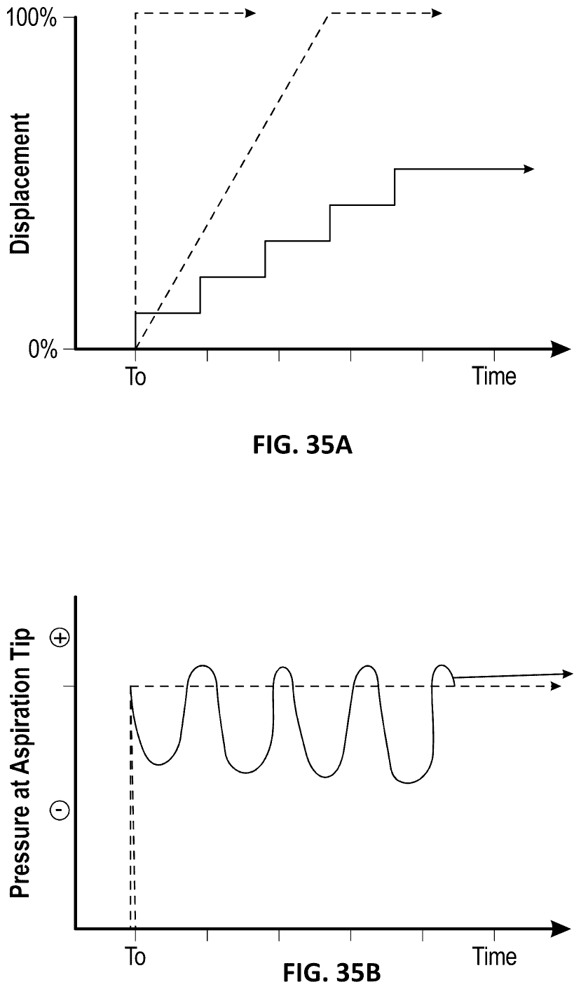

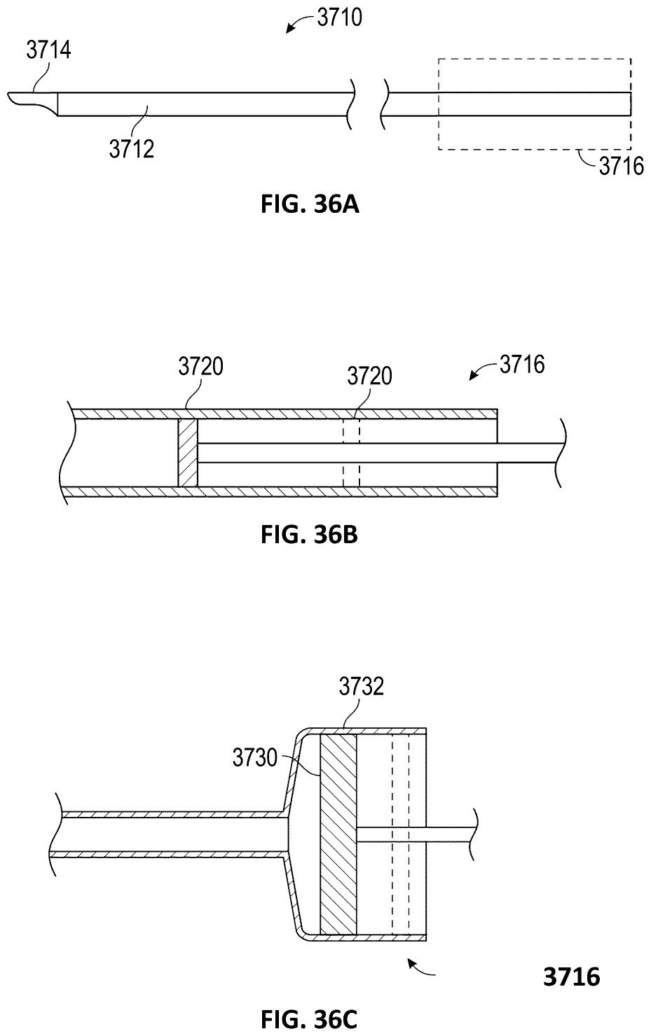

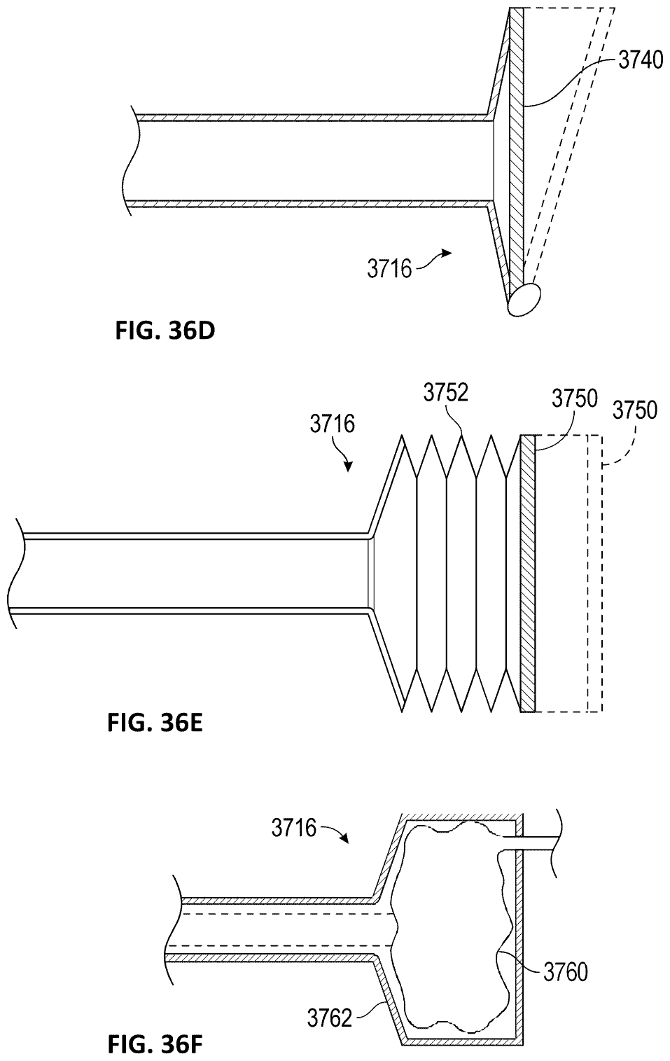

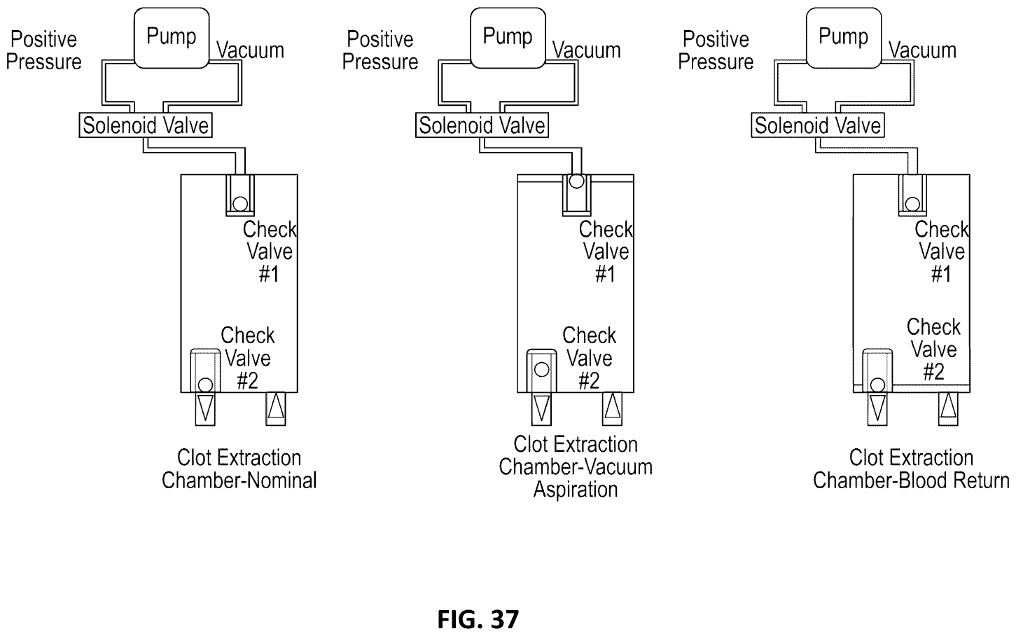

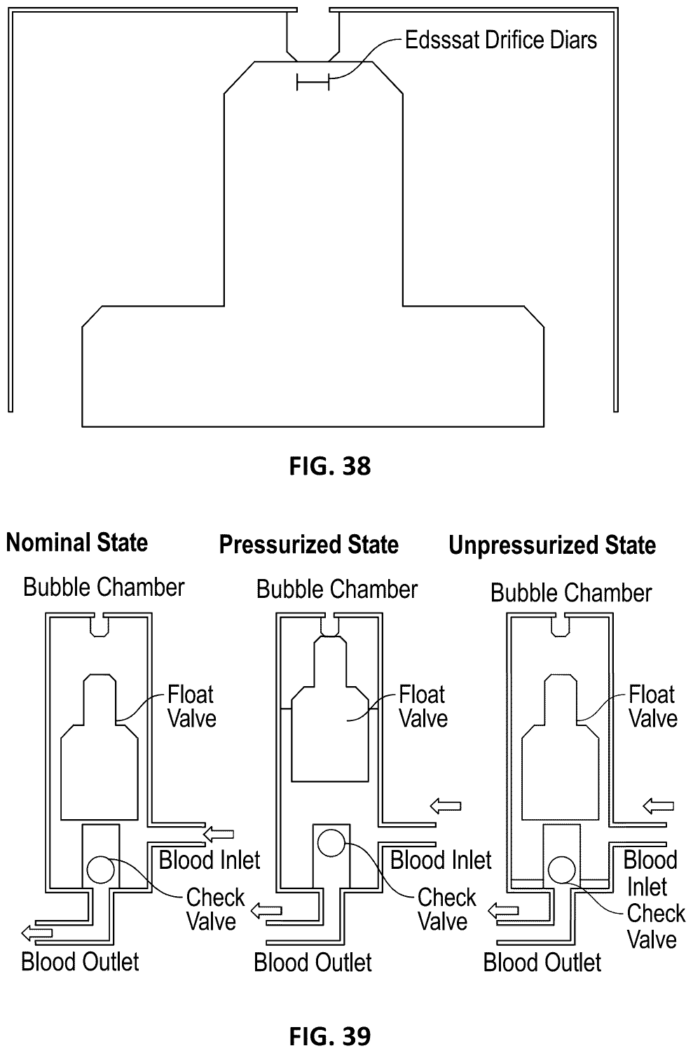

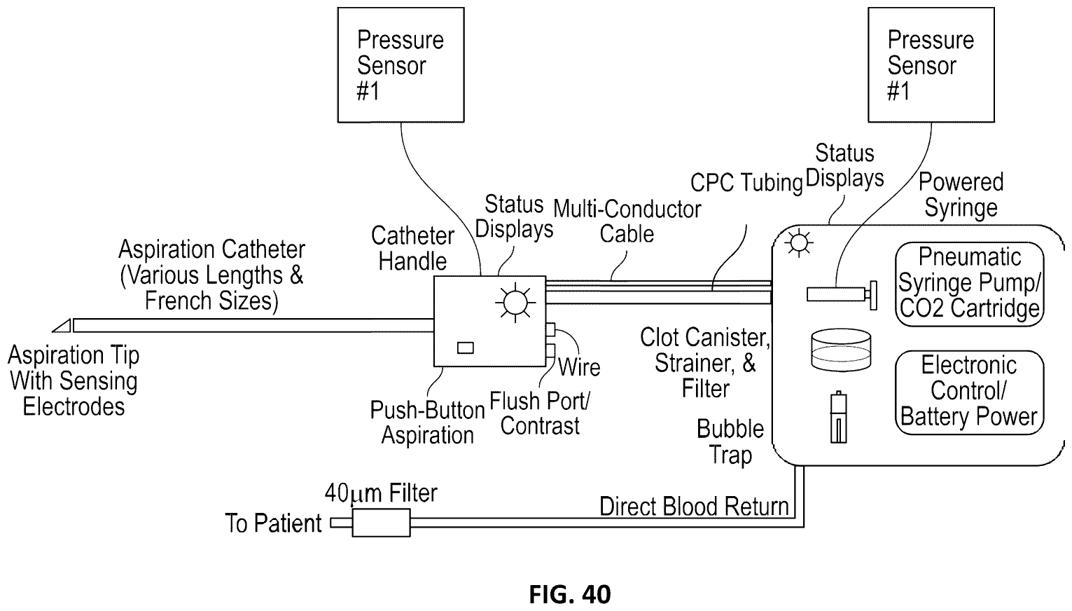

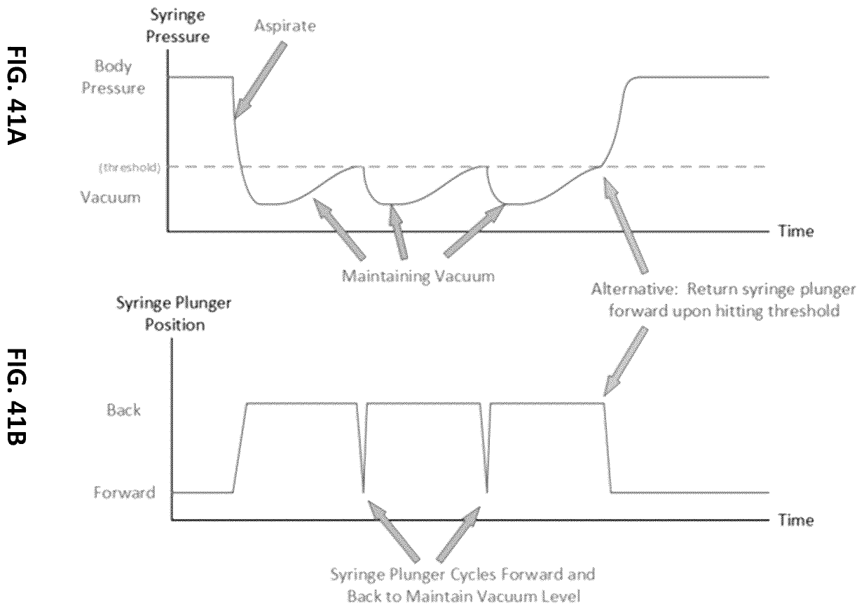

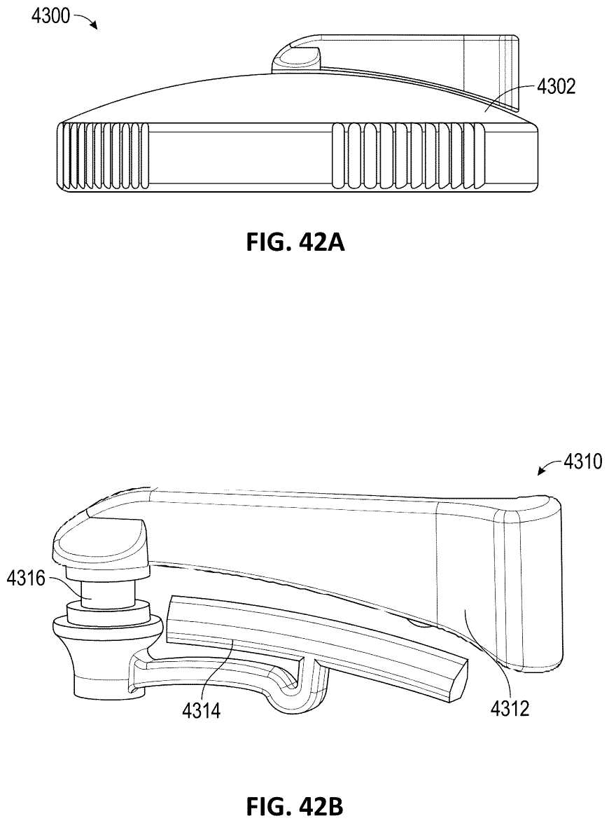

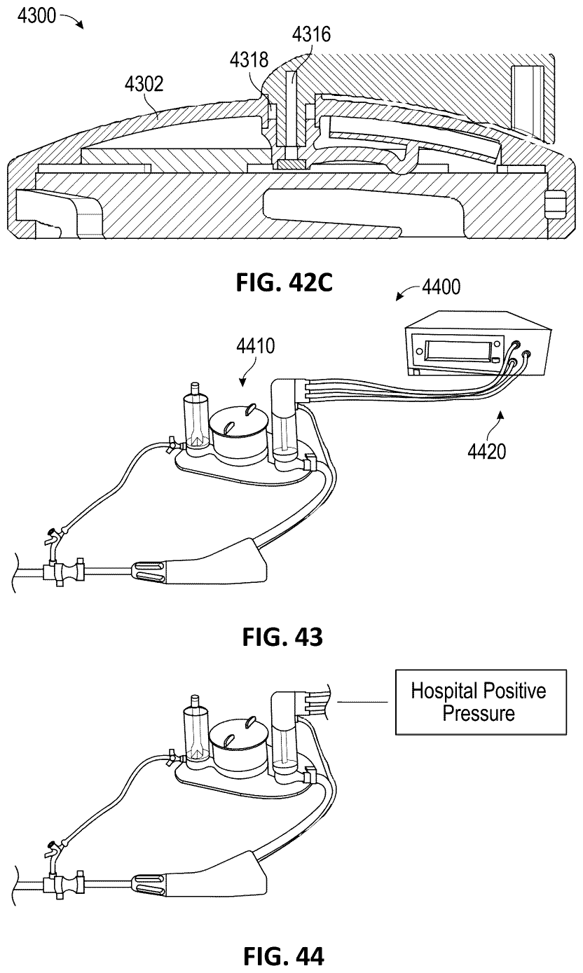

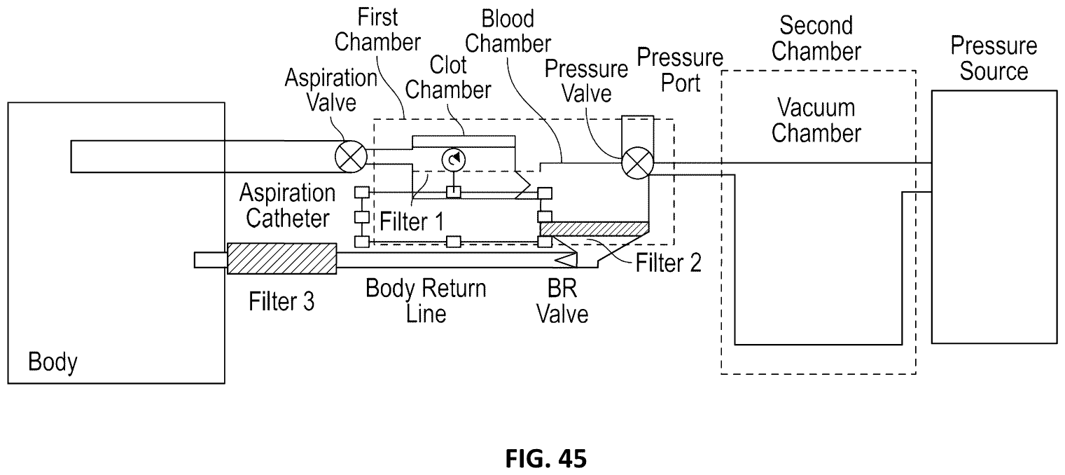

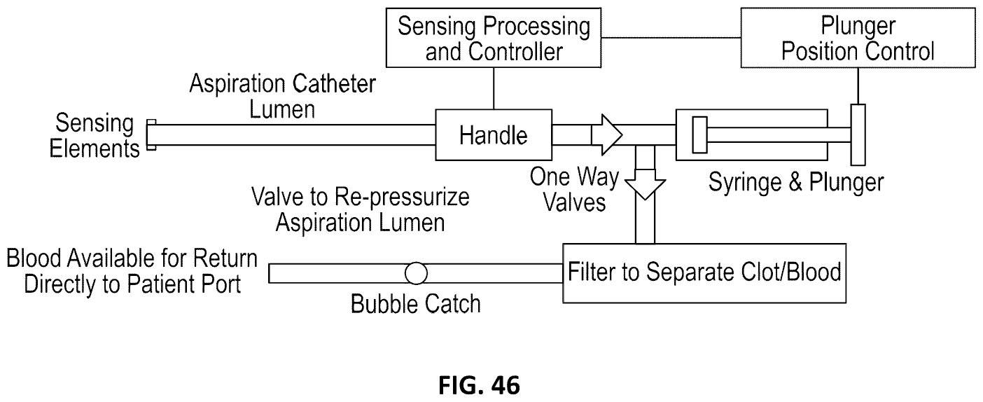

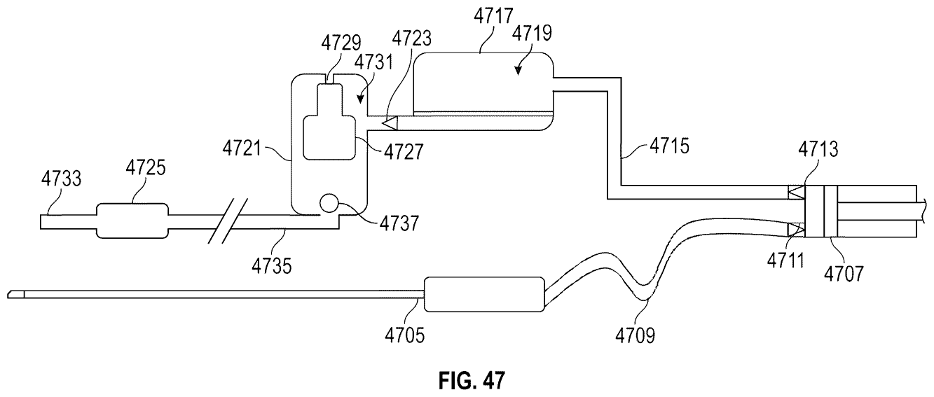

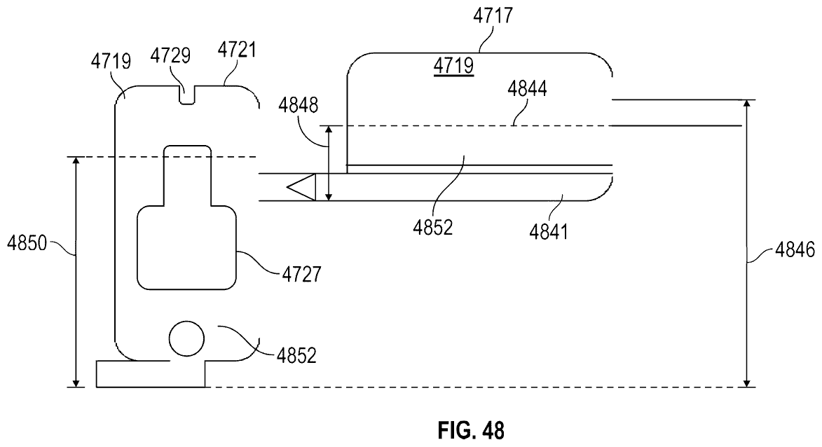

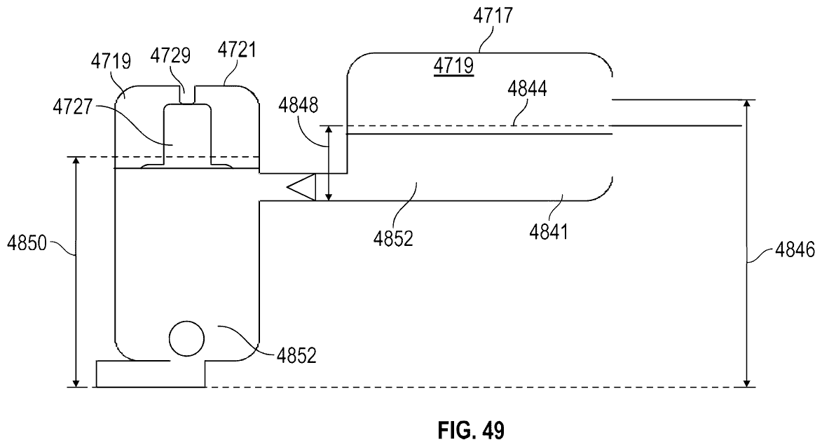

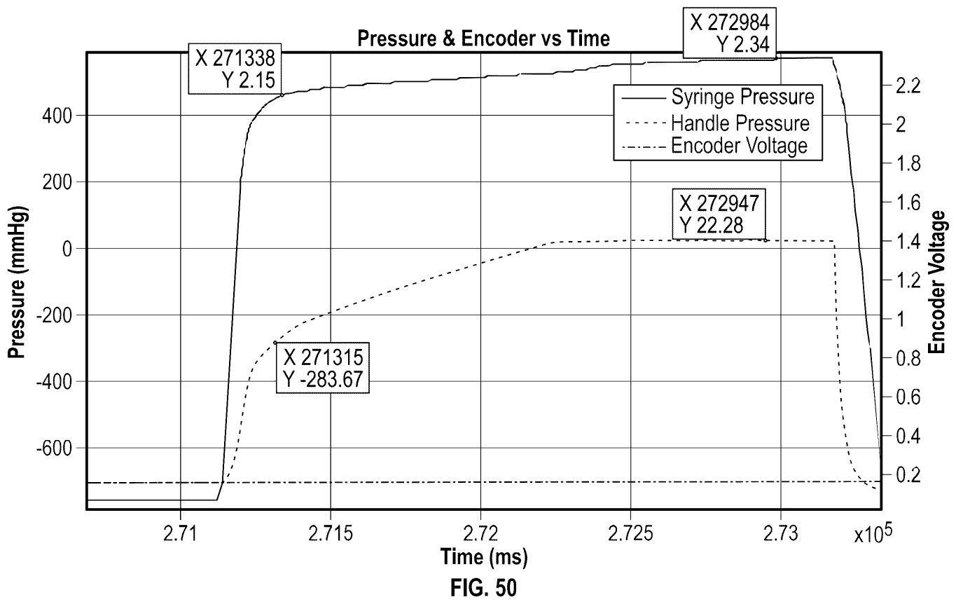

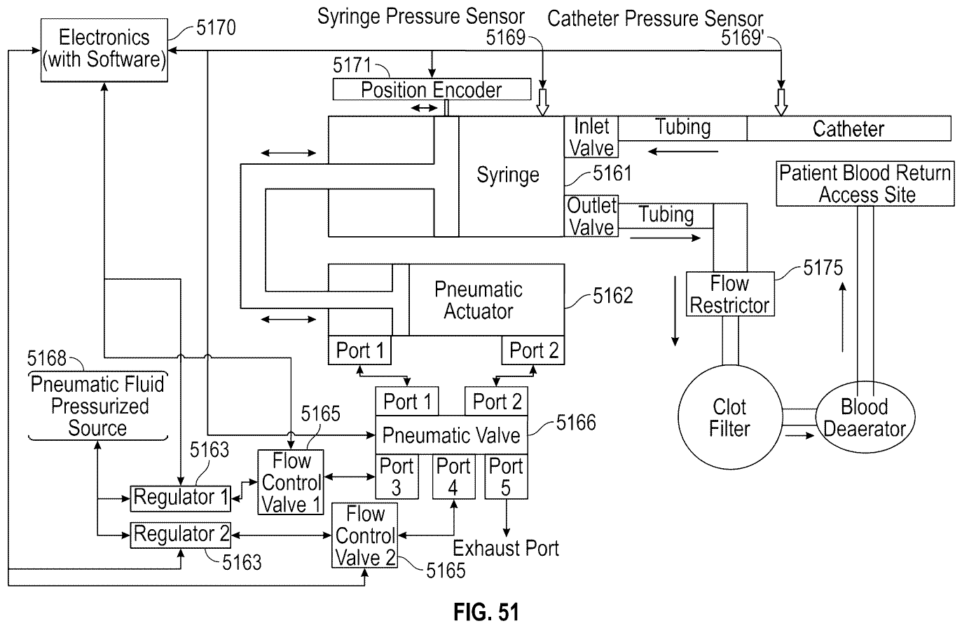

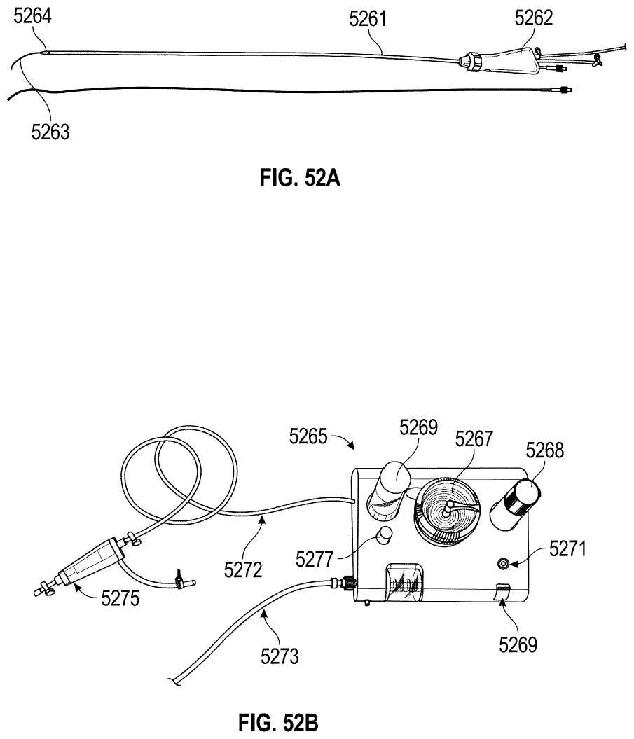

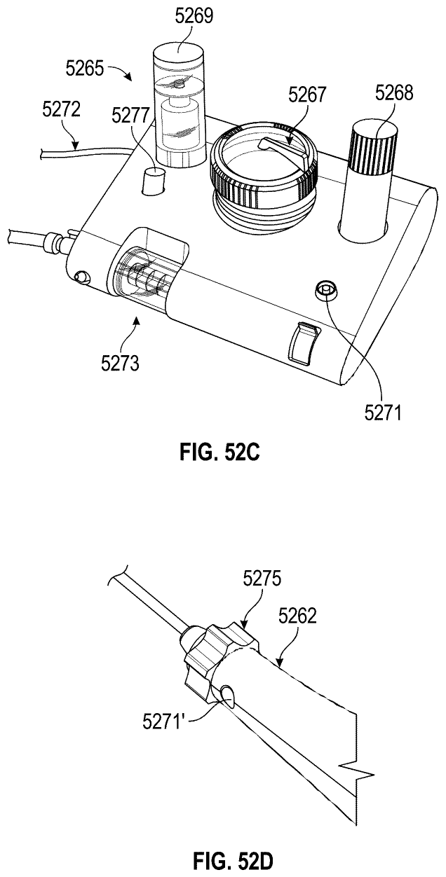

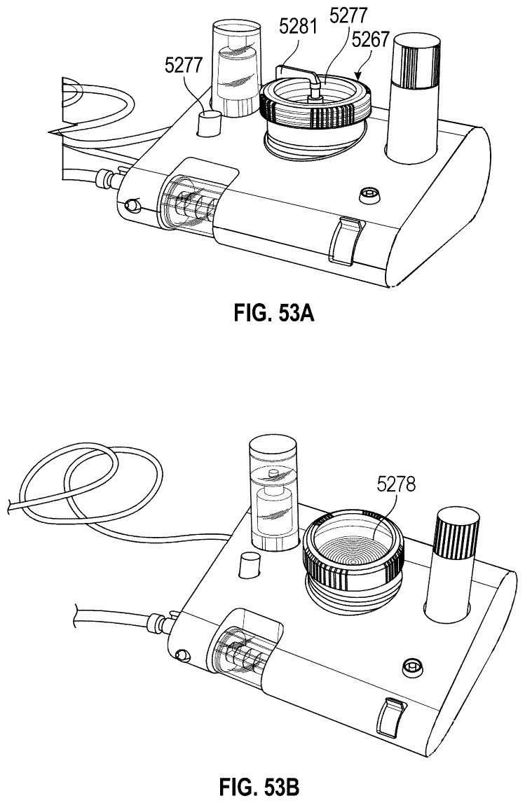

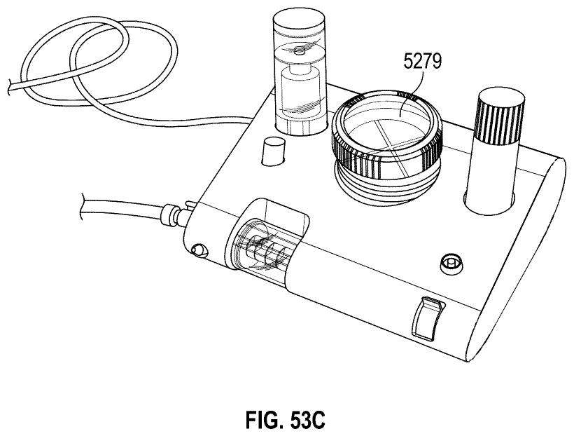

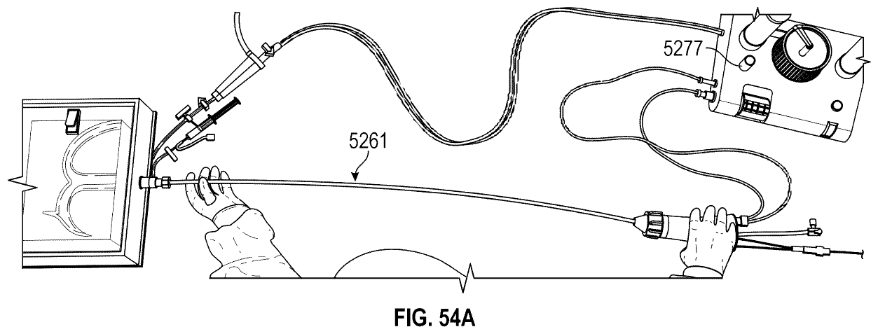

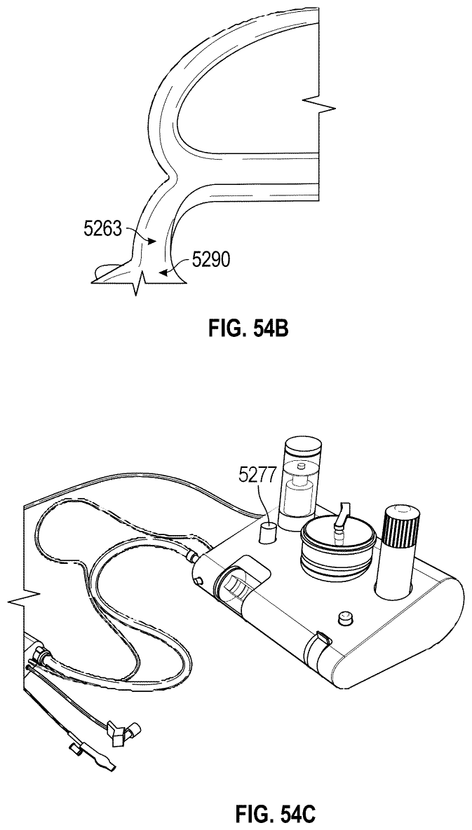

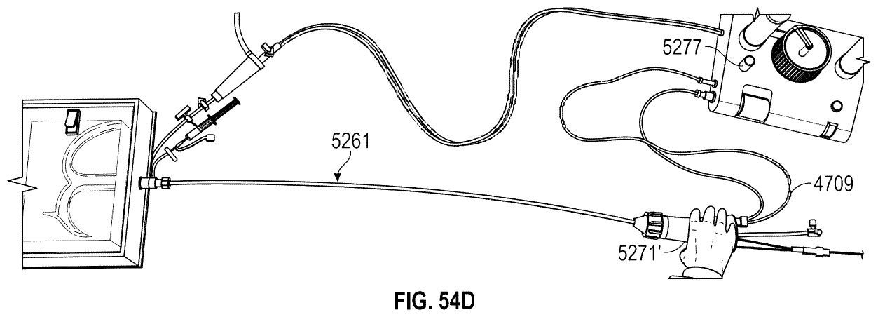

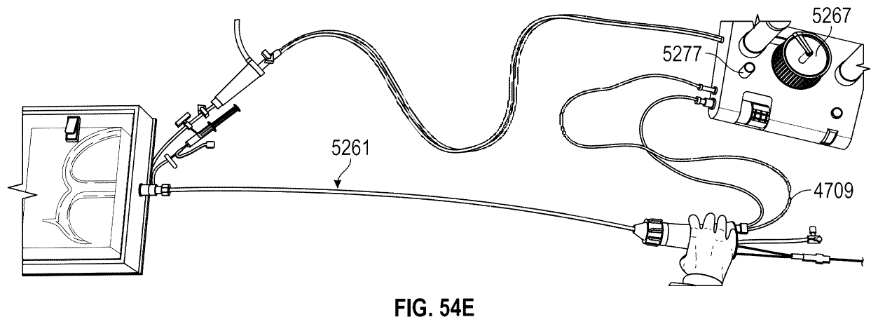

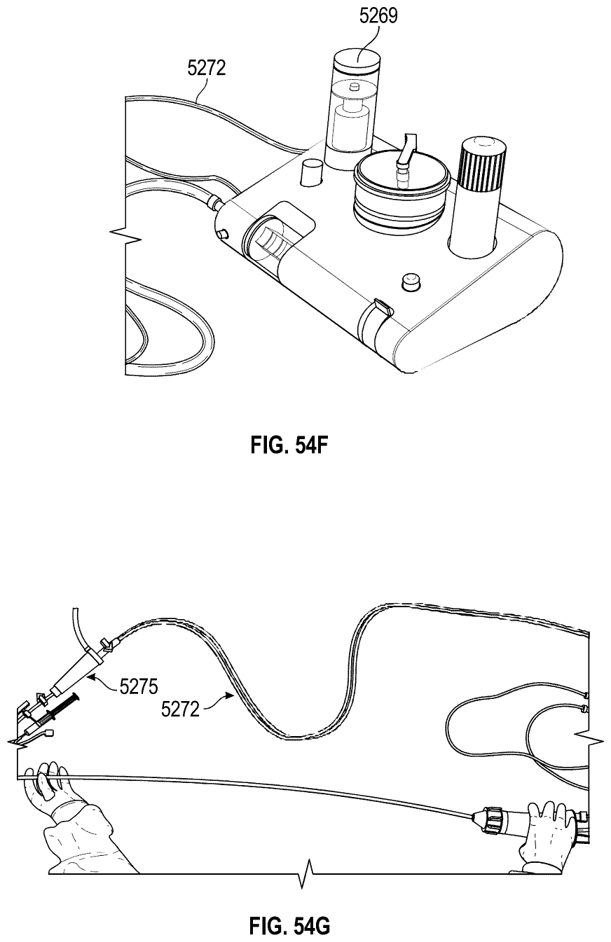

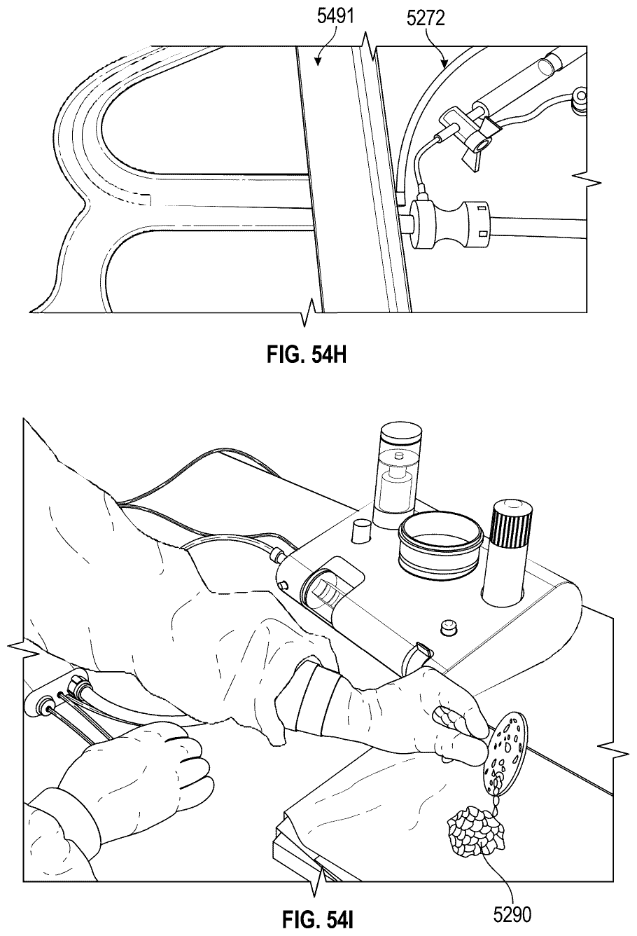

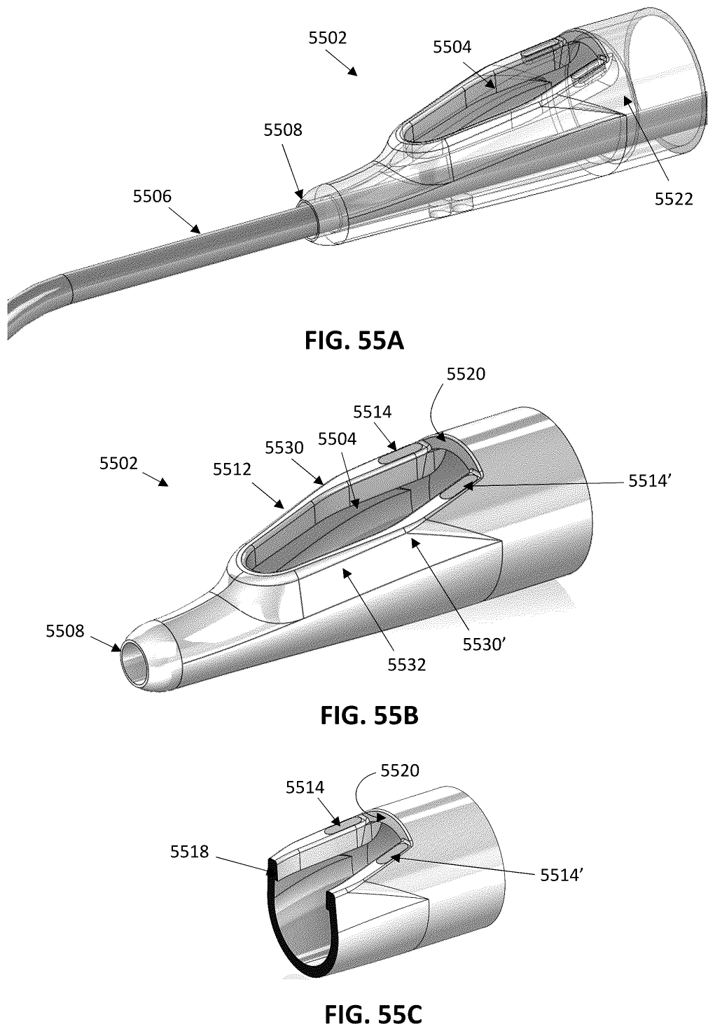

FIG. 6J illustrates a double action aspiration syringe with pneumatic motion driver system in accordance with the disclosed technology.

FIG. 7 schematically illustrates an example of a system including a hinge-valve in place of one-way valves for flow control, to allow for oscillation motion of the syringe plunger.

FIG. 8 illustrates the use of an oscillation-type motion of the syringe plunger in a system with a hinge-valve.

FIG. 9 schematically illustrates an example of a sensing thrombectomy system with blood return as described herein.

FIG. 10 shows an example of an automatic sensing thrombectomy system with blood return.

FIG. 11 schematically illustrates an example of a manual, disposable fluidic-driven aspirator. This example illustrates embodiments that utilize compressed gas and/or springs to drive an aspirator piston to aspirate blood, clot, and other obstructive materials from a patient's vasculature utilizing the disclosed technologies.

FIG. 12 schematically illustrates an example of a disposable fluidic-driven aspirator configured to include a sensing control valve.

FIG. 13 shows one example of a disposable fluidic-driven aspirator including a manual control (e.g., button), showing additional embodiments that utilize compressed gas and/or springs to drive an aspirator piston to aspirate blood, clot, and other obstructive materials from a patient's vasculature utilizing the disclosed technologies.

FIG. 14 is a graph illustrating fluid volume moved by compressed canister weight.

FIGS. 15 and 16 illustrate alternative fluidic driver constructions which have one fluidic drive compartment and one spring return compartment.

FIG. 17 shows an alternative blood return path for the system of FIG. 12.

FIG. 18 shows an alternative flow regulator placement for the system of FIG. 12.

FIG. 19 shows an example of a timing diagram of two simple plunger position algorithms.

FIG. 20 illustrates alternative plunger control algorithm timing diagrams.

FIG. 21 illustrates operation of a small syringe with full stroke, shown at a fast rate, exceeding the max fill rate dictated by the aspiration lumen and 1 atm of vacuum

FIG. 22 demonstrates the plunger continuing to aspirate after the completion of sensing the clot at the tip of the catheter.

FIG. 23 schematically illustrates one example of a system having additional sensing areas.

FIG. 24 schematically illustrates a multi-syringe arrangement.

FIG. 25 schematically illustrates an example of a system using blood return to re-pressurize an aspiration lumen.

FIGS. 26 to 32 illustrate embodiments of combined filtering and deaeration chambers which can be utilized to filter and de-aerate blood utilizing the disclosed technologies.

FIG. 33 schematically illustrates of a system for controlling aspiration pressure in accordance with the disclosed technology.

FIG. 34 is a flowchart illustrating exemplary method steps for controlling aspiration pressure using the system of FIG. X1 in accordance with the disclosed technology.

FIG. 35A is a graph of displacement element position vs. time for several representative operational protocols of the disclosed technology. Charged aspiration (Dashed line: ---------); Controlled aspiration (Broken line: _-_-_-_-); Stepped aspiration (Full line: ______).

FIG. 35B is a graph of chamber pressure vs. time for the operational protocols of FIG. 34.

FIGS. 36A to 36F illustrate alternative embodiments of the disclosed technologies incorporating an expansion chamber in an aspiration catheter.

FIG. 37 illustrates an alternative pressure control and filtering assembly that can be utilized in the disclosed technologies.

FIG. 38 illustrates a float valve that can be utilized in the deaerators of the disclosed technologies.

FIG. 39 illustrate operation of the float valve of FIG. 38 in a deaerator of the disclosed technologies.

FIG. 40 illustrates an automatic clot aspiration systems having two pressure sensors for detecting blockages and other flow conditions.

FIGS. 41A and 41B are comparison graphs plotting pressure and syringe position versus time.

FIGS. 42A to 42C illustrate an exemplary wiper configuration suitable for use in the blood filters of the disclosed technologies.

FIG. 43 illustrates an aspiration system according to the disclosed technology having reusable pump and electronic components.

FIG. 44 illustrates an aspiration system according to the disclosed technology configured to run from an external positive pressure supply.

FIG. 45 illustrates a further alternative embodiment of an aspiration system according to the disclosed technologies.

FIG. 46 schematically illustrates an example of a system with automatic blood return and a bubble catch component.

FIG. 47 schematically illustrates a closed-loop clot removal and blood return circuit that is configured to withdrawal blood from a patient's body at a rate that does not depend on the rate that blood is returned to the patient's body.

FIG. 48 schematically illustrates a portion of a blood removal and return circuit that includes a clot collection chamber with a capacitive air reservoir that is configured to hold a minimum volume of air. The schematic shown in FIG. 48 does not include blood.

FIG. 49 schematically illustrates the portion of a blood removal and return circuit of FIG. 48 with blood present.

FIG. 50 is a graph showing a relationship between pressure (in mmHg), encoder voltage and time (in milliseconds) for one example of a piston-type pump (e.g., syringe) as described herein.

FIG. 51 schematically illustrates one example of a system (including a blood removal and return circuit) that is configured as a smart fluidic drive system.

FIGS. 52A-52D illustrate one example of an apparatus (e.g., a system) including or forming a blood removal and return circuit.

FIGS. 53A-53C illustrate details of the clot collection chamber of the apparatus shown in FIGS. 52A-52D.

FIGS. 54A-54I illustrate operation of the apparatus of FIGS. 52A-52D.

FIGS. 55A-55E illustrate one example of distal end region of an aspiration catheter.

FIGS. 56A-56B illustrate one example of a proximal handle portion of an aspiration catheter.

### DETAILED DESCRIPTION

Described herein are thrombectomy apparatuses (e.g., devices, systems, etc. including hardware, software and/or firmware) for removal of clot material from the body. In particular, these apparatuses may be configured to return blood removed from the patient as part of the clot removal process, back into the patient. In some cases these apparatuses may be closed-loop apparatuses forming a “blood circuit,” in which blood and clot material is removed from the body, filtered and otherwise processed to separate out the clot material and to prepare the blood for re-introduction into the patient, and then returning the blood into the patient. Alternatively these systems may be configured just to remove clot material and blood from the patient. In some cases blood may be removed from the patient using all or some of these components described herein, and blood may be returned in a manual or semi-manual manner.

Any of the thrombectomy apparatuses described herein may include an aspiration device to apply negative pressure (e.g., suction) to remove clot material, and in some cases blood, from the patient. These aspiration devices may be included as part of a system, such as a blood return system, with any one or more of the thrombectomy apparatuses described herein, or may be provided on their own, and/or for use with off-the-shelf components, such as aspiration catheters, etc. For example, the aspiration devices describe herein are configured to apply suction, e.g., to an aspiration catheter, to remove clot material (and blood) from the patient through an aspiration catheter. In particular, described herein are aspiration devices that include a fluidic driver that is driven by positive pressure, and in particular stored positive pressure, to provide on-demand aspiration (e.g., negative pressure). The aspiration devices described herein may also be referred to as pumps, or as aspiration devices having a fluidic driver. These aspiration devices may provide relatively high immediate flow rates without requiring a stored vacuum. Further, these aspiration devices minimize damage to the blood, including minimizing exposure to vacuum, and prevent or reduce the introduction of bubbles into the blood.

Blood filtering devices are described herein. These devices may include one or more filters (filtering elements). A blood filtering device as described herein may be referred to equivalently as a blood filtering chamber or simply a filtration chamber. In particular, described herein are blood filtering chambers that are configured to provide enhanced visualization of, and in some cases may provide access to, clot material removed from the body. In some cases, these blood filtering chambers may be configured to operate without disrupting the blood circuit. For example, a blood filtering chamber may include one or more visualization windows that allow visualization of clot material. In some cases the visualization window(s) may be cleared, e.g., wiped, using an internal wiper without opening the chamber. The blood filtering devices described herein may be provided as part of a system, e.g., a blood return system, including for use with any one or more of the thrombectomy apparatuses described herein, or they may be used with off-the-shelf components.

Also described herein are deaeration (or “de-airing”) devices for removal air bubbles from blood within the blood circuit, e.g., prior to returning the blood to the patient. These deaeration devices may include one or more chambers (and may be referred to equivalently herein as deaeration chambers or de-airing chambers) or portions and may be vented to regulate the air pressure within the device. A deaeration device may be combined with or integrated with one or more blood filtering chambers. The deaeration devices described herein may include a capacitive air reservoir that is configured to hold a minimum volume of air between the viewing window and the filter. This capacitive air reservoir may provide a compressible region that enhances the operation of the device, including preventing retrograde flow within this region of the blood circuit and/or enhancing operation of the filtration within the filtration chamber when coupled to the deaeration device. The deaeration devices described herein may be used as part of a system including one or more of the thrombectomy apparatuses described herein (e.g., aspiration devices having a fluidic driver, blood filtering devices, etc.) and/or they may be provided on their own, and/or used with other components.

The apparatuses described herein may include one or more controllers for controlling and/or coordinating the operation of the devices or a system including one or more of these devices. A controller may include one or more processors and memory and may be configured to execute instructions (e.g., programs), receive input from a user (e.g., doctor, nurse, technician, etc.), and provide output to control operation of one or more components, such as an aspiration device, as well as to communicate operational parameter and/or statue with the user. In some cases the controller may include controls for regulating operation of flow through a blood circuit by regulating operation of an aspiration device. In some examples the controllers described herein may be an aspiration pump controller for controlling operation of an aspiration devices that includes a fluidic driver.

As mentioned, the apparatuses described herein may include one or more sensors, including but not limited to sensors for sensing blood flow, clot material, blood, etc. For example, any of these methods and apparatuses may include sensing or detecting the clot material and/or blood at one or more parts of the blood circuit, including one or more of: the aspiration catheter that is configured to remove the clot material and blood, the aspiration line removing clot material and blood, one or more filters, a blood collection chamber, a deaeration chamber, an aspiration device (e.g., pump), and a blood return line. Sensing may include, but is not limited to pressure sensing, electrical sensing (e.g., impedance sensing, capacitance sensing, etc.), optical sensing, etc.

Any of the components described herein, including the aspiration devices, blood filtering devices, deaeration devices, fluid lines, controllers, etc. may be included as part of an apparatus, e.g., system, for removing clot material and blood from a patient, removing clot material from the blood, and for returning blood to the patient. Any of these apparatuses may optionally include an aspiration catheter or may be configured for operation with an aspiration catheter. In general, these apparatuses may be configured to apply suction (e.g., negative pressure) to remove clot material and blood, and positive pressure to drive the clot material and/or blood thought the blood circuit (including the blood filtering device, deairing devices, etc.). For example, described herein are apparatuses including one or more aspiration lines (e.g., tubing), an aspiration device configured to apply aspiration, a clot collection chamber, a deaeration device, and a blood return line configured to return processed blood back into the patient.

### Aspiration Devices

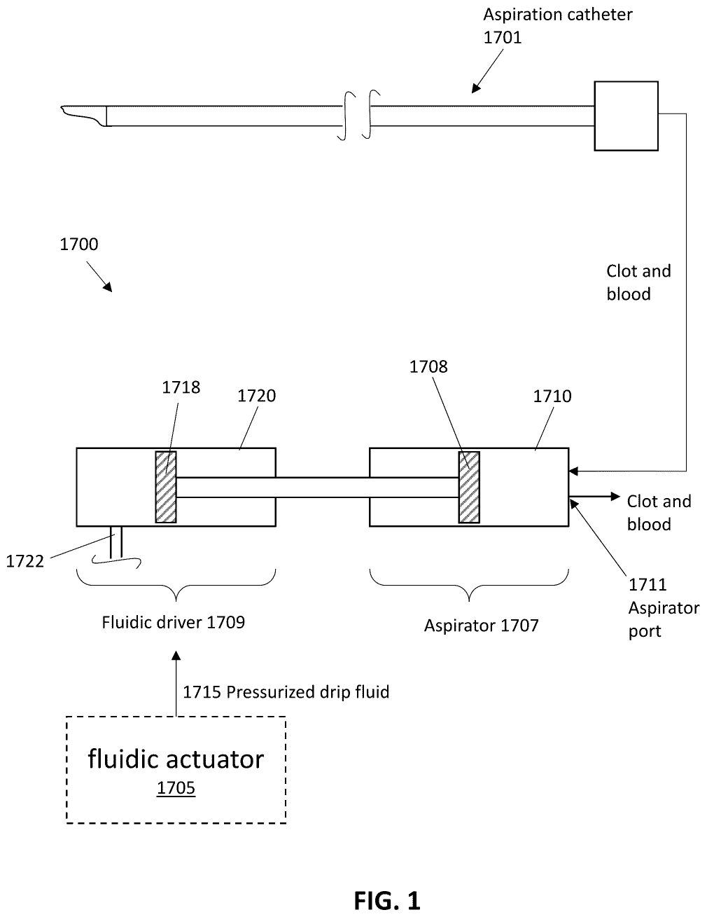

Any of the methods and apparatuses described herein may include one or more aspiration device, and in particular, aspiration devices including a fluidic actuator. For example, FIG. 1 schematically illustrates an example of an apparatus (e.g., system) as described herein. The aspiration device 1700 may be used with an aspiration catheter 1701 and a fluidic actuator 1705 configured to deliver a pressurized drive fluid. The apparatus (e.g., aspiration device) in this example includes an aspirator 1707 including an aspirator displacement element 1708, an aspirator chamber (e.g., aspirator cylinder) 1710, and an aspirator port 1711. The aspirator port is configured to connect to an aspiration lumen of the aspiration catheter 1701. The device also includes a fluidic driver 1709 including a driver displacement element 1718, a driver chamber (e.g., driver cylinder) 1720, and at least a first fluid port 1722 configured to receive the pressurized drive fluid from the fluidic actuator, wherein the pressurized fluid causes the driver displacement element to translate in a first direction and wherein the aspirator displacement element is coupled to travel in tandem in the first direction with the driver displacement element to draw blood and clot through the aspirator port and into and from the aspiration cylinder, when a distal port of the aspiration catheter is in a patient blood vessel proximate clot.

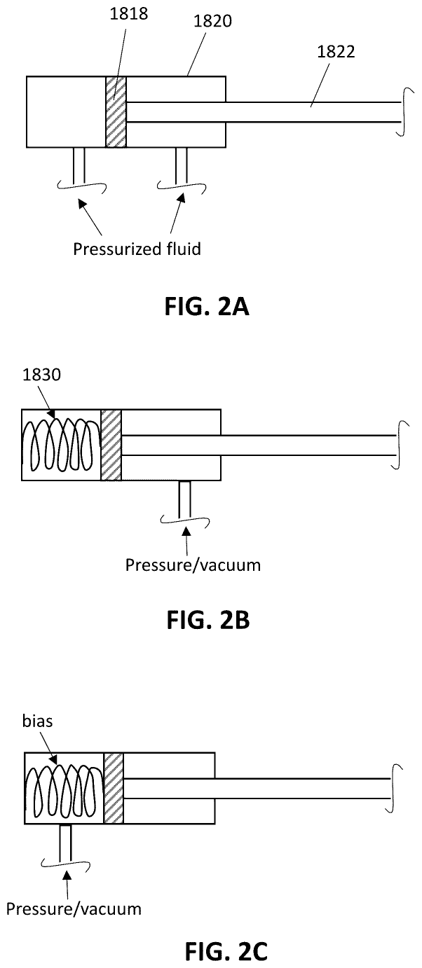

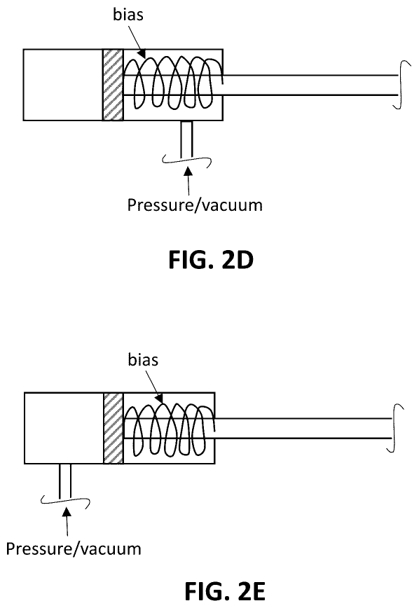

A variety of different fluidic drivers may be used, including, but not limited to, those shown in FIGS. 2A-2E. As shown in FIGS. 2A-2E, the fluidic driver may generally include a driver displacement element 1818, a driver chamber (e.g., driver cylinder) 1820; the drive displacement element (e.g., piston, surface, cylinder, disk, slider, etc.) may be coupled to a transmission (e.g., rod, shaft, etc.) 1822 for coupling to the aspirator (e.g., the aspirator displacement element (such as an aspirator piston, surface, cylinder, disk, slider, etc.). In FIG. 2A the fluidic driver includes a first chamber region having a variable volume on a first side of the drive displacement element, and a second chamber region having a variable volume on the second side of the drive displacement element. By changing the pressure within one or both of the first and second chambers, the drive displacement element may be controllably moved and positioned as described above. In FIG. 2A both the first chamber and the second chamber receive input from a pressurized fluid; the first and second chambers may be separately pressurized (with positive and/or negative pressure). Alternatively one chamber may include a pressure release (e.g., vent) while the pressure of the other chamber is adjusted.

In some examples either or both chambers may include a bias applying force to the drive displacement element. For example, in FIG. 2B the first chamber includes a bias 1830 (e.g., spring) and may be vented or may be separately pressurized; the second chamber may include an input for a pressure (e.g., pressurized fluid, positive and/or negative pressure, etc.). In FIG. 2C the same chamber that includes the bias also includes an input for the controlling pressure (e.g., pressurized fluid, and/or positive and/or negative pressure, etc.). The bias may be in the second chamber, as shown in FIGS. 2D and 2E.

The drive displacement element may be configured to have a very low friction, as described above. For example, either the drive displacement element and/or the inner surface of the drive chamber may have a lubricious surface, providing a very low static friction between the drive displacement element and the chamber. For example, the coefficient of static friction between the drive displacement element and the chamber may be selected to be less than 0.15 (e.g., 0.1 or less, 0.9 or less, 0.8 or less, 0.7 or less, 0.6 or less, 0.5 or less, 0.4 or less, 0.3 or less, 0.2 or less, 0.1 or less, etc.).

In general, there are many ways in which mechanical forces can be applied to move the aspirator plunger to achieve the features of this invention. Key features may include: 1) moving the plunger at a rate that generates a pressure differential between the plunger face and the distal aspiration opening of an aspiration lumen at least greater than 10 mmHg and preferably greater than 600 mmHg, 2) the ability to finely control this pressure differential to achieve maximum flowrates through the aspiration lumen when desired and minimize fluid removal when desired, 3) the ability to know and control precisely the plunger position to enable controlled fluid movement, such as 1, 5, 10, 15, 20, 30, 60 cc of fluid movement at a time. These incremental plunger movements can be user defined or controlled by the controller of the system based on at least one measured output from the system or the procedure. In any of the ways in which the plunger is controlled, it may be beneficial for the system to have independent knowledge of the current position, such as through a position encoder, resistive sensor, or other means. Another consideration when providing mechanical power for plunger positioning is the sterile barrier in a Cath Lab, which should be maintained, and which can be burdensome to handle in some implementations.

One way to provide the mechanical force to move the plunger is a linear stage electric motor (either a stepper motor or brushless DC motor) with a rotating drive shaft that crosses the sterile barrier. The drive shaft may either be a long and flexible torque cable, or a rigid member. The rotational motion may be converted to the linear plunger motion using leadscrew, rack and pinion, or other rotational to linear translation arrangements.

Another way to control the plunger is by compressed air/pneumatics. Compressed air may be used to push the plunger backwards to generate vacuum and forward to generate a positive pressure. The piston volumes may be chosen to be equal to the max stroke volume desired in the syringe use, or a larger piston may be used, and programmable-position stops may be implemented to adjust the end-positions with more precise control than just bursts of compressed air. Pressures, compressed fluid flowrates, pneumatic piston dimensions, syringe barrel dimensions, and travel distance of the plunger can all be chosen to meet or exceed the key features described above. For the use of aspirating blood and clot, the syringe barrel inner-diameters shall be larger than the inner diameter of the aspiration lumen. An example of a syringe barrel range may be about 0.6-5 cm, which may ensure the obstructive can flow into the barrel while requiring a reasonable force to move the plunger.

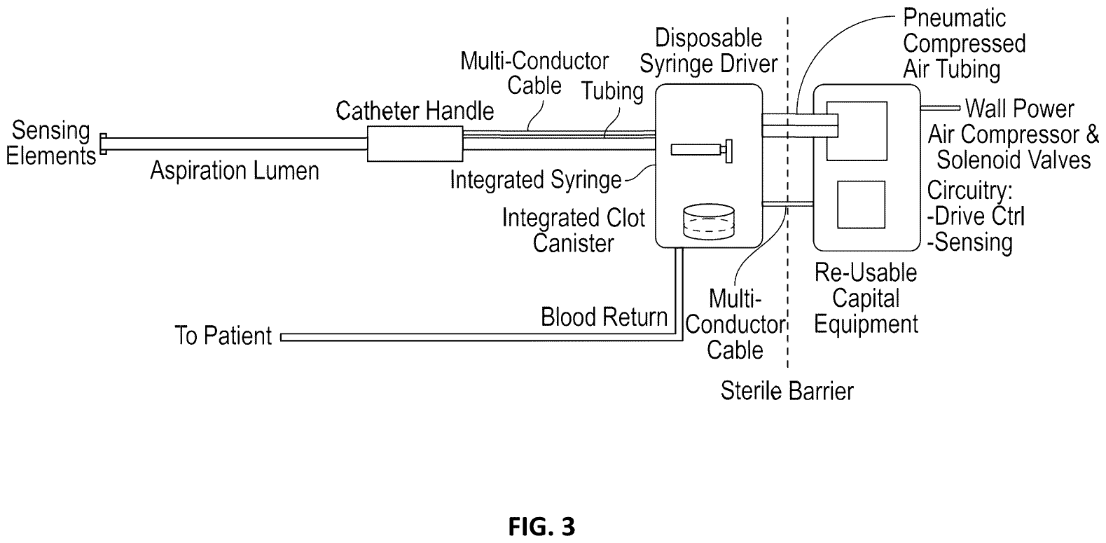

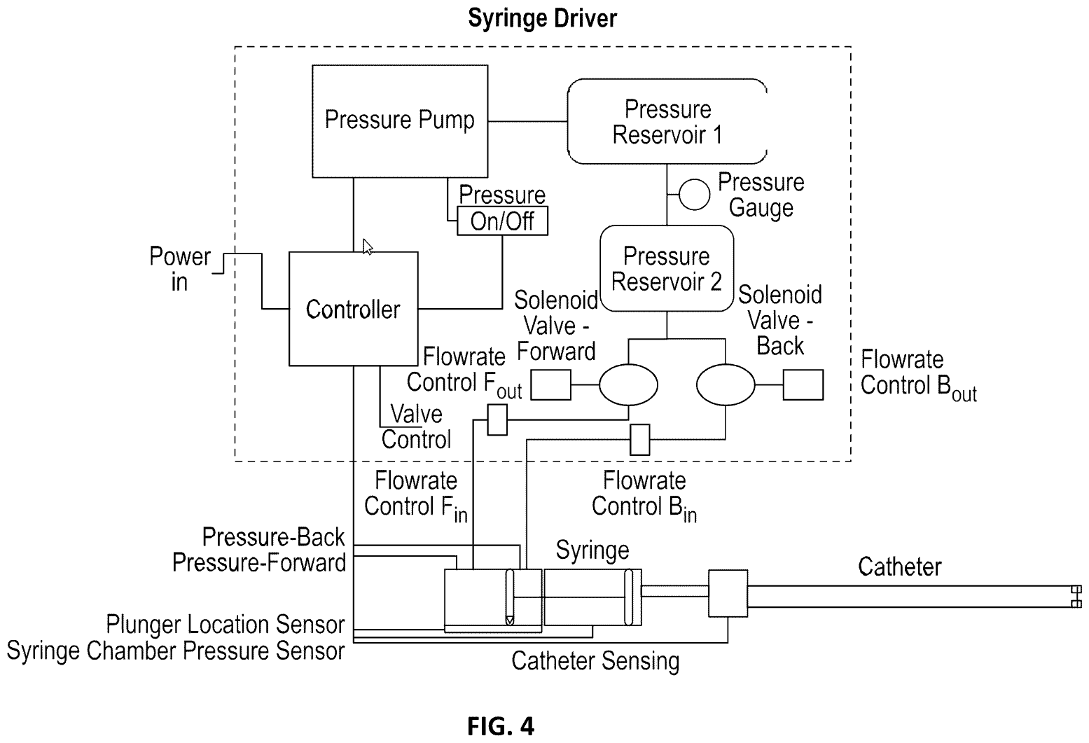

Another option is to intentionally meter-down the applied pressure using needle valves or similar, to slow down the motion and intentionally limit the flowrate to values less than the max achievable through the aspiration lumen, in either the aspiration direction or in the fluid return direction. This is useful to help prevent vessel collapse, vessel wall latch, as well as reduce hemolysis of the blood as it passes through the system which can occur when the blood is exposed to high velocities and turbulent action. One benefit of a fluidic driven syringe is that the sterile barrier is crossed simply by two air-tubes and a wire for sensing. System design diagrams are shown in FIGS. 3 and 4. FIG. 3 schematically illustrates one example of a pneumatically powered system showing the sterile barrier. FIG. 4 shows an example of a pneumatic internal operation diagram for control of syringe plunger.

Note also that the endpoints of plunger position under pneumatic control need not be just the ends of the syringe size. Programmable stop points may be implemented which can either physically stop the plunger at several different programmable locations for good accuracy (such as a leadscrew stop), or known bursts of air could be used to perform less-than full piston motion. The air flow can either be implemented as the ‘single-pole-double-throw’ type of operation where one and only one side of the piston is pressurized at a time, or independent control may be used which gives much more flexibility regarding arbitrary position control.

In any of the pneumatically controlled plunger position implementations, it is highly advantageous to utilize a fluidic piston as well as a syringe plunger with very low frictional forces to minimize the static forces required to overcome to initiate moving of the plunger. A small static friction force may allow for quick responsiveness of the system and to allow fine-motion control.

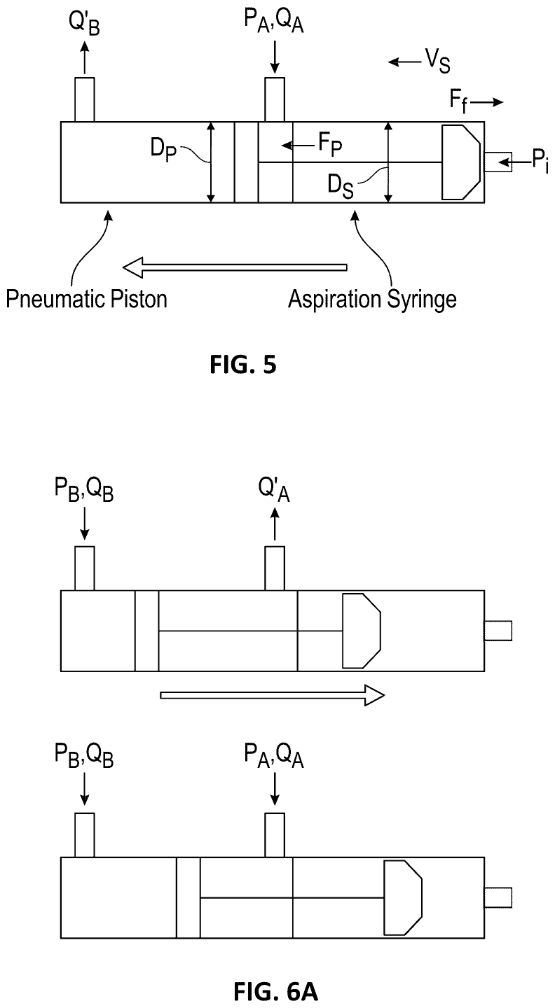

More details about how a syringe plunger position may be controlled using pneumatics and a mechanically coupled piston are shown in FIGS. 5 and 6A-6J. The pressures required to cause piston motion may be implemented using positive pressure (such as compressed air), the piston motion may also be accomplished using vacuum applied to one side of the piston or the other, on its own or in combination with positive pressure. Overall, a differential pressure across the two sides of the piston may cause the motion. With regards to FIG. 5, to achieve a certain pressure Pi with a syringe with a barrel diameter Ds, a force Fp must be applied by the pneumatic piston:

$${F}_{P} = ( {P}_{i} / ( π * {( {D}_{s} )}^{2} ) ) + {F}_{f}$$

wherein F_{f} is the frictional force of the syringe barrel and plunger. In addition, it is important for the pneumatic piston to be able to move the syringe plunger at a certain velocity (Vₛ) and Force (Fₚ) to achieve the desired flow rate within the catheter. The velocity of the pneumatic piston is a function of volumetric flow rate of air (QA) from the compressor as well as the piston area (a function of Dₚ). The force the piston applies is a function of the pressure applied by the compressor (P_{A}) and area (a function of Dₚ) of the piston. Simply put:

$${Q}_{C} = f ( {F}_{f} , {D}_{S} , {D}_{P} , {P}_{A} , {Q}_{A} )$$

wherein Q_{c} is the catheter flow rate; Fr is the frictional force between the plunger and syringe barrel; Dₛ is the inner diameter of the syringe; Dₚ is the inner diameter of the piston; P_{A} is the pressure applied to the piston; and Qₐ is the flow rate into piston. In addition, Q′_{B} is the flow rate out of the B side of the pneumatic piston (venting). Restriction Q′B will decrease the velocity at which the piston can retract. With regards to FIG. 6A (first position), to limit hemolysis, it may be desirable to reduce the flow rate of blood out of the syringe. To do so, either P_{B} or Q_{A} can be limited, or the flow rate out Q′_{A} from chamber A of the pneumatic can be restricted. Doing either of these will slow the forward motion of the piston. With regards to FIG. 6 (second position), it may be desirable to stop the piston mid stroke. To achieve this P_{A} and P_{B} must be equal. The rate at which they achieve equilibrium will be a function of Q_{A} and Q_{B}.

Having independent control of the valve rates (not just on/off but control of flow rates) into and out of each of the sides of the drive piston allows for arbitrary position control of the piston and therefore the syringe. This rate control may be useful to help reduce hemolysis of the blood which is damaged when exposed to vigorous physical conditions. The controlled flow rates may be static or dynamic based on one measured output from the system or the procedure such as pressure, flowrate, piston location, and/or material at distal opening and/or within of the aspiration lumen. Complicated algorithms which involve not just simple back and forth motion, but which incorporate oscillation as well as overall suction, may be implemented in this way. A different valving system may be used in this case, to allow for the oscillation to take effect through the whole fluid column of the aspiration lumen, while still allowing for a separate return path. A ‘hinge’ type valve may be used in this case, which flips to the return position when the syringe is being pushed forward. See FIGS. 7 and 8. FIG. 7 shows an example of a system including a hinge-valve in place of one-way valves for flow control, to allow for oscillation motion of the syringe plunger. illustrates the use of an oscillation-type motion of the syringe plunger in a system with a hinge-valve.

An additional benefit of using pneumatics for piston position control is that the compressed air line may be used for other functions within the sterile field. Actions such as a valve controlling/metering blood return to the patient may be powered using the pneumatic capabilities. Also, other actions such as physically manipulating/macerating the clot may be powered in this way (although maceration of the blood/clot may render the blood non-returnable). Similarly, the pneumatics could control a contrast-injection/power-injector, which could be enabled by a button-press by the user.

Another way to control the plunger motion is through hydraulics. With the same signal inputs as a pneumatic system as described above to determine when to drive the movement of the plunger, the pump to move the hydraulic fluid may be in the durable (reusable) equipment out of the sterile field, with a set of hydraulic lines coming into the sterile field in a sterile bag. The other layout could be to have the lines be in the disposable and sterilized portion and may be connected to the durable out of the sterile field at the time of prep, with a prescribed de-airing procedure. The advantages of hydraulics over pneumatics are that the motion can be much more precisely controlled and can be controlled much faster, due to the incompressible nature of the hydraulic fluid instead of the compressible air.

Another way to control the plunger motion is through user-input force such as manually pulling the syringe plunger or through a pedal that would be depressed by the user. In both of these scenarios, the force and linear plunger retraction rates would be user controlled based on the user interface information on the handle or other part of the system indicating the state of the tip sensors. If the user is informed that a clot is present at the tip of the catheter, they may act by either pulling the plunger or depressing a pedal which would drive the retraction of syringe plunger. This method may give the user more freedom over how much, how fast, and how frequently to aspirate in conjunction with the sensing information provided.

Additional options for using human-power includes a ratcheting syringe, which stops at known sip sizes, and is charged up by manual pulls. Mechanical advantage to help lower the manual force requirements may be implemented using cams, levers and other mechanisms to increase distance and lower force.

### Powered Syringe Shot Size Selector

In aspiration based venous thrombectomy clot and blood are removed from the body using a vacuum source. Certain systems may use the retracting motion of a large bore syringe to create negative pressure and remove blood and clot. It is desirable that the volume of aspiration be as small as possible to effectively remove the clot and minimize blood loss. However, a large range of aspirations volumes may be required if targeting clots in the pulmonary arteries (60 cc or greater) vs the peripheral leg veins (<15 cc). If an aspiration with too small of a volume is used in a large vein or against a large clot, it may be ineffective. If an aspiration with too large of a volume is used in a small vein, it may collapse the vessel and be ineffective as well. Therefore, a mechanism is needed in which the user can adjust the volume of blood and clot being aspirated during the procedure.

The mechanism shown in FIGS. 6B, 6C, 6D, 6E, 6F, 6G, and 6H which show examples of a 60 cc syringe 600 with a 1.5″ diameter barrel, powered by a pneumatic piston. However, the syringe size could vary anywhere from 10 cc to 100 cc or greater. Additionally, the power source for the syringe could be motor, hydraulic, or manually driven. In this mechanism, the plunger 607 of the syringe is attached to a carriage. This carriage is attached to the pneumatic piston which powers the syringe. The carriage also has several ledges. When the pneumatic piston retracts the syringe plunger, a ledge of the carriage will hit a pin attached to a knob. This creates a retraction hard stop. If the user rotates the knob a specified distance, the pin will land on a different ledge. These ledges are spaced such that correspond to different volumes. In this embodiment, the ledges correspond to 60 cc, 30 cc, and 15 cc retractions. However, one skilled in the art could see how increasing the number of ledges would increase the resolution of volume selection.

The knob itself has a detent (a ball and spring 610 in this embodiment), which clicks into place at the specified volume selections. This gives the user feedback that the selection has been correctly made. Alternatively, the knob could be mechanically linked to a rack and pinion type mechanism such that the user slides a control to change the volume.

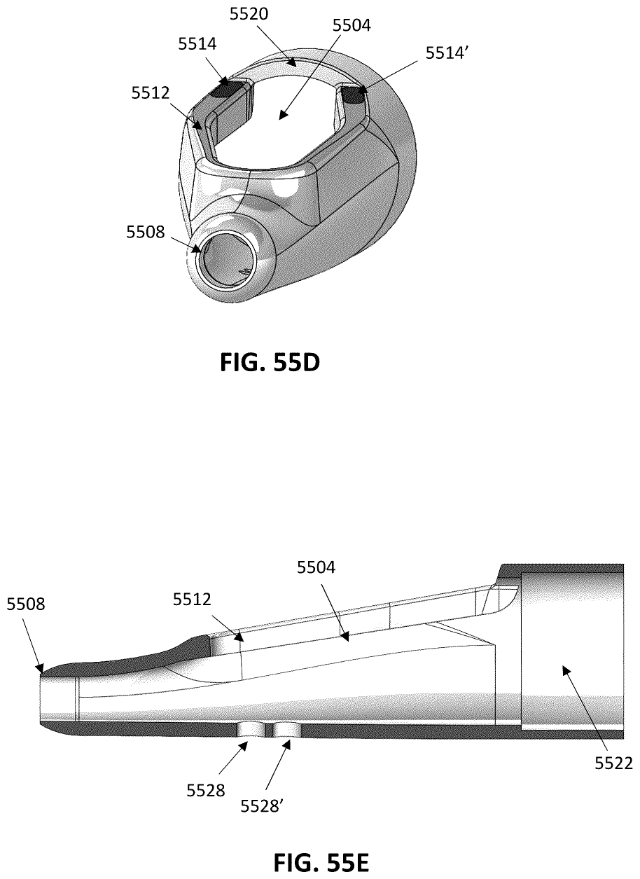

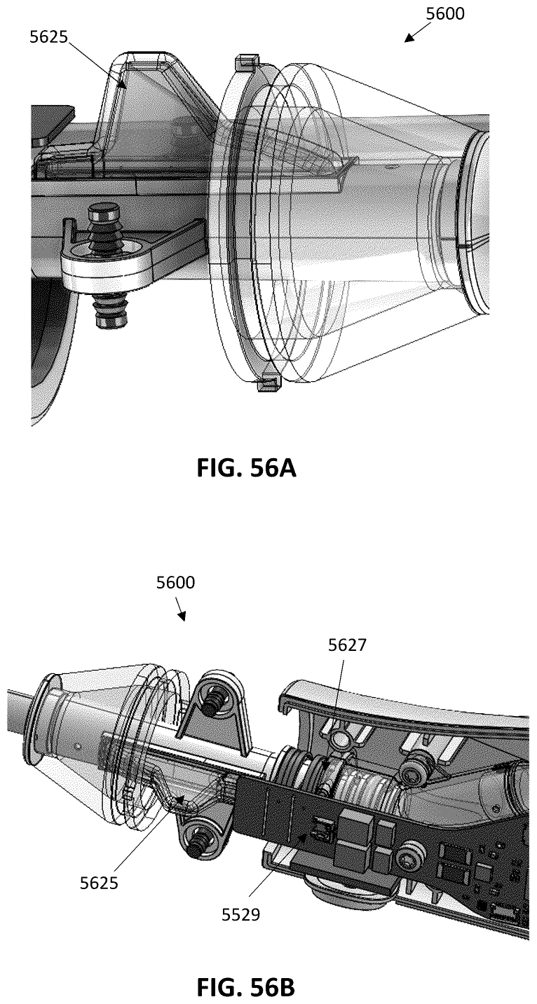

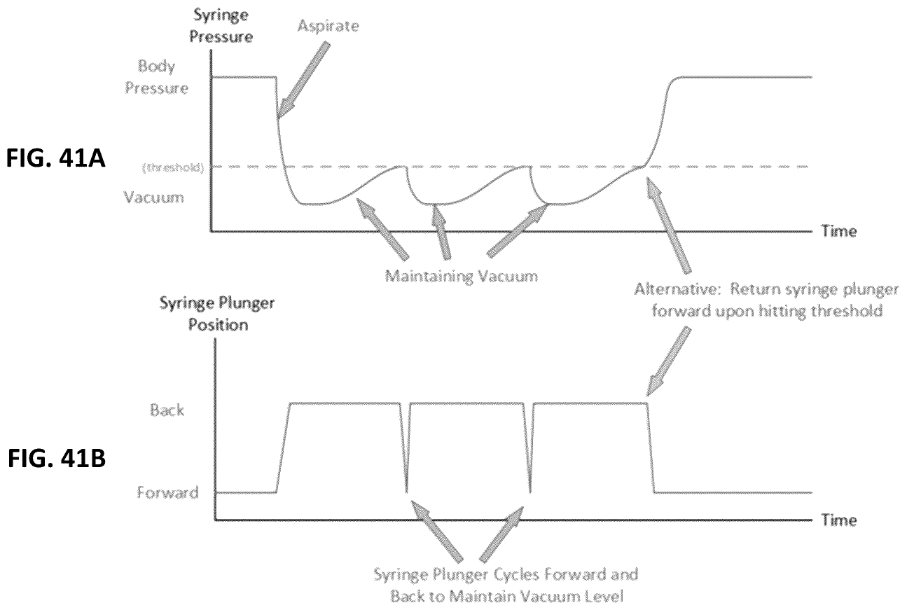

Although in this embodiment, the user manually turns the knob, this knob could be rotated by a stepper or servo motor. This would allow for electronic control of volume by the user. Alternatively, this volume control could be integrated into the algorithm for clot sensing. If the catheter sensed a large clot, it could automatically set the syringe to a larger volume, and vice versa. FIGS. 61 and 6J illustrates a double action aspiration syringe with pneumatic motion driver system in accordance with the disclosed technology.

### Powered Syringe Size Variants

As previously described above, there is desire for the user and/or system to have different shot sizes for different aspiration scenarios. In the figures and embodiments above, the 1.5″ syringe barrel was used to demonstrate the 15 cc, 30 cc, and 60 cc shot size examples. Each of those volumes represents a different stroke length of 0.52″, 2.62″, and 5.24″, respectively. From both performance and/or user-experience perspectives, there may be a need for a different syringe barrel and stroke length relationship to accommodate more or less shot size volume in a given system. The advantages of changing the stroke length and barrel diameter relationship may allow for different systems to be optimized for different needs, which may include, but are not limited to the following:
shot size volume,
shot size resolution,
overall form factor,
force required to retract and/or return the syringe plunger,
force required to achieve a certain syringe plunger retraction and/or advancing rate,
efficiency, and
power required to achieve different aspiration rates and/or volumes.

One example may utilize a 1″ syringe barrel internal diameter, which would equate to stroke lengths of 1.16″, 2.32″, and 4.64″ for the same 15 cc, 30 cc, and 60 cc volumes, respectively. The barrel diameter may be decreased or increased, and the stroke length of the plunger would therefore increase and decrease respectively to achieve the same volumes. However, in adjusting the syringe barrel diameter the forces needed to move the syringe plunger and therefore the forces exerted on the aspirated fluid differ.

Using the relationship that pressure is proportional to force and area, and that the maximum achievable vacuum in a syringe-based aspiration system is 1 atmosphere (˜14.7 PSI), then the force required to generate 1 atm of vacuum by a syringe is proportional to the surface are of the syringe. In the case of a 1.5″ syringe, the surface area is 1.77 square-inches, and therefor assuming 1 atm is 14.7 pounds per square-in, then the force required to generate 1 atm of vacuum with a 1.5″ barrel should be 25.9 pounds. If the syringe diameter is changed to 1″ or 2″, that force changes to 11.5 pounds and 46.2 pounds, respectively. The syringe barrel diameter and the required forces needed to move that syringe barrel, may have a significant impact on the type of syringe motion driver that is selected, such as, but not limited to: the use of a magnetic solenoid, coupled or decoupled electric motor, and/or a pneumatic air cylinder. In addition to the force needed to move the syringe plunger, there may be requirements for the clinical performance, that a certain minimum aspiration rate be achieved from the aspiration syringe.

In a syringe-based aspiration thrombectomy system, the maximum instantaneous and average aspiration flowrates of the system are based on the ability of that system to deliver vacuum to the tip of the catheter and on how much resistance to flow that system has. Given that power is directly proportional to pressure by the following relationship: Power=Pressure×Flowrate, and that the maximum achievable vacuum to aspirate is a fixed pressure; the main contributor to that system's aspiration power is the rate at which that vacuum pressure can be delivered (i.e., Flowrate) to the tip of the catheter. Using the understanding that pressure equals force over area, and that in order to move fluid a pressure differential must be present; therefore, for a fixed diameter the more force that is applied to that fluid, the more that fluid will accelerate, and higher flowrates can be achieved. The higher the flowrates that can be achieved, the more powerful the aspiration system, assuming that aspiration system is able to generate vacuum as its power source. For a syringe-based aspiration source that develops vacuum as it retracts, the ability of that syringe plunger to exert a force on the fluid with which it is interfacing is directly proportional to the force driving that syringe plunger motion. For a syringe with a smaller diameter, the force needed to move the fluid into that syringe at a given flowrate will be lower than the force needed to move fluid into a larger diameter syringe at the same flowrate. Alternatively, if the same force is applied to two syringes; one of a smaller diameter than the other, than the flowrate of the fluid into the syringes will be different: the smaller syringe will have a higher flowrate.

In a system using a pneumatic air cylinder as a source of power to drive syringe motion, the air cylinder may be a different and/or the same diameter as the syringe barrel in order to tune the pneumatic pressure required to operate that system. In the example shown in FIG. 1, the air cylinder is a smaller diameter than the syringe barrel, so the operational pneumatic pressure is proportionately higher than the vacuum pressure. Depending on the design of the system and its components, the operational pneumatic pressure may be limited in operational pressures including, but not limited to the following reasons: cost, complexity, reliability, efficiency, material properties, safety, and/or a combination of those factors. In the case that the air cylinder is the same diameter as the syringe barrel, then in a frictionless system, the pneumatic pressure of the air cylinder would be the same as the vacuum force (˜14.7 PSI) to move the syringe plunger. To account for the frictions in the system, the air cylinder diameter may be increased, or the pneumatic pressure increased to account for that frictional increase in forces.

In the case of a syringe-based aspiration system that desires maximum aspiration power, then the aspiration source must provide as much and as fast as possible the vacuum force to the catheter tip. In that case, then the rate at which the syringe retracts as it is generating vacuum, and the syringe diameter are important contributors to the ability of that system to generate maximal aspiration power. However, the more the syringe diameter and syringe retraction rate are increased, the more power will be needed to generate that motion. In the case of the pneumatic air cylinder motion driver-based system, the rate and pressure at which the air, CO2, Nitrogen, and/or not limited to any other gas type used to drive the air cylinder will be directly proportional to the ability of that system to drive syringe plunger rates and forces. In order to maintain rapid air cylinder rates, large diameter fittings and pneumatic lines should be used, as well as fast switching pneumatic controls, in order to further increase performance.

### Direct Coupled Aspiration Syringe Pneumatic System

In the event that a syringe-based aspiration thrombectomy system is desired, as described above there are various ways to generate vacuum and fluid motion, however in one embodiment, the syringe plunger may be directly coupled to the pneumatic air cylinder. This allows for separation between the patient fluid contacting components and the pneumatic media contacting components, while optimizing the complexity, number of components, and reducing frictional forces within the system as shown in FIG. 6I.

Double Action Aspiration Syringe with Pneumatic Motion Driver System

In the event that a syringe-based aspiration thrombectomy system is desired, as described above there are various ways to generate vacuum and fluid motion, however in one embodiment, the syringe plunger may be directly coupled to the pneumatic air cylinder and drive fluid motion in both directions of stroke travel. This allows for separation between the patient fluid contacting components and the pneumatic media contacting components, while optimizing the duty cycle of the system to achieve an aspiration and a return on every stroke. As pneumatics pressurizes one side of the pneumatics cylinder, it drives motion of the plunger coupler and due to the inlet flow control element on one side of the syringe barrel, blood/clot are aspirated through the catheter, while the other side of the syringe is being pushed out the outlet flow control element to expel previously aspirated clot/blood to the clot filter. This system is shown in the diagram in FIG. 6J.

### Inclusion of Positive Pressure or Venting

In some cases, the physical characteristics of the clot in the vessel may require additional maneuvers and forces to coax it into the aspiration orifice for effective extraction. One such method is the inclusion of positive pressure alternating with the application of vacuum, to create a jack-hammer action at the catheter tip. There are several ways in which this positive pressure may be achieved, including:

The “oscillation” type system mentioned above, using advanced plunger position algorithms to both oscillate the fluid column and perform aspiration, and use a hinge-valve to allow for blood return in a separate path.

A “leaky” one-way valve coming from the aspiration lumen to the syringe, such that a small amount of fluid will push back into the lumen before sealing. This is essentially designing in hysteresis in the valve. This way, each time the plunger is pushed forward we get a positive pressure wave out at the tip of the catheter.

The amount of fluid that is pushed each cycle (see hysteresis above) may be programmable and based on system measurements such as pressures.

A system of valves may be used to connect the aspiration lumen to a positive pressure of fluid, or to outside atmospheric pressure.

Positive pressure may also come from the blood return line, which could split to have a line back towards the aspiration lumen. This may prevent the need for additional saline fluid introduction and prevents air entry into the system which reduces subsequent vacuum performance. The positive push of the plunger and pressurization of the blood-return portion of the system could provide a short positive pressure back to the aspiration lumen to help with the jack-hammer effect of oscillating pressure for improved clot extraction.

Plunger-positioning algorithms that pull the plunger back to perform aspiration include small oscillation motion back and forth along the way, to create the pulsing effect.

Systems with two syringes, one main syringe to perform the vacuum extraction and another smaller syringe may be used to oscillate a small amount of fluid to create the pulsing effect.

A system which takes advantage of the self-resonance of the fluid system, which when exposed to a step function in pressure may self-oscillate as the pressure wave travels through the system at a frequency determined as a function of the capacitive (chamber sizes) and inductive (fluid inertia) properties of the design. The resonance may be utilized by timing the opening/closing of valves, or pulling syringes with prescribed sip sizes and timing, such that a positive oscillation pressure wave may be exerted at the tip even without explicitly causing a positive pressure in another fashion.

### Disposable Fluidically Driven Aspirator

The minimally invasive removal of unwanted material from the body may be performed anywhere in the body. In many of the procedures where unwanted material is removed there is a need for preserving the fluid (e.g., blood) that is also removed, e.g., from within that part of the body. An example of this is thrombectomy, as discussed above. Thrombectomy is the removal of thrombus (blood clots) from various parts of the human vasculature. Several types of systems are currently in use, including manual aspiration with a syringe, aspiration via vacuum-pump with and without computerized valve control and/or with and without clot maceration, and physical scraping/catching of clot from the vessel and vessel walls. Existing systems have shown that mechanically removing of clot either via aspiration or physical force is effective; however, there are safety concerns, and the systems are inefficient from a blood loss, user cognitive and physical exertion, and procedure time.

Systems which include a vacuum chamber and a vacuum-valve to connect the aspiration lumen to the vacuum chamber suffer from hemolysis of the blood, which renders it unreturnable to the patient. This occurs when the blood is exposed to open vacuum/air for extended periods, which also causes the blood to foam and de-gas. Procedures with these types of systems tend to incur excessive blood loss and may have to prematurely stop the procedure due to blood loss concerns. These systems also require a capital piece of equipment which makes setup more difficult and removes controls from the user in the sterile field as those controls must be placed out of the sterile field.

Systems which require manual operation for clot extraction are ill-suited to peripheral thrombectomy procedures such as deep-vein thrombosis (DVT) because the clot burden needed to be removed could be quite large. Using these systems require many repetitive steps to sufficiently remove the clot burden. This includes both manual aspiration systems as well as systems which physically pull the clot from the vessel. For manual aspiration systems though burdensome due to the repetitive steps, the aspiration syringe design does achieve the highest pressure differentials while reducing the time the blood is exposed to the vacuum pressure which enables effective clot burden removal while maintaining the quality of the blood to allow the blood to be returned.

The methods and apparatuses described herein may address these shortcomings and may provide an improved way for generating negative pressure and positive pressure within a cylinder for the movement of fluid and unwanted material from the body and/or into a collection chamber. In some embodiments of the invention, the fluid removed is filtered and able to be returned back to the body. In further embodiments, the filter fluid is returned directly back to the body using the positive pressure generated by the invention. The improvements described herein may include: reduced procedure time, reduced number of required operators, and reduced effort needed by the user to generate negative pressure by eliminating the need to connect and disconnect the pressure source or having to exert a force on the aspiration source, such as pulling and pushing on a syringe. These methods and apparatuses may also eliminate the need for capital equipment. These methods and apparatuses may also provide for auto-activation of the pressure source based on procedure feedback. In some cases these methods and apparatuses may enable on-demand user activation of pressure source that resides all in the sterile field. These methods and apparatuses may also increase control of the whole system pressure source and put the controls in the sterile field to enable the user easy access. These methods and apparatuses may also eliminate procedure steps when moving fluid and material from and to the body and may provide features that can sense and determine what is at the aspiration opening of the aspiration catheter. In some cases, these methods and apparatuses may also provide sensing for monitoring for air and clot within the return fluid going back to the patient.

These improvements may be achieved by using mechanical forces to move an aspirator piston within a cylinder at a rate to generate a pressure differential that can adequately move fluid and unwanted material. The methods and apparatuses may be configured to fluidly connect to an aspiration lumen of a catheter. This may allow control of the rate at which the system generates a pressure differential between the aspirator piston face and the distal aspiration opening of an aspiration lumen at least greater than 10 mmHg and preferably greater than 600 mmHg as well as controlling the rate of the aspirator piston forward. The driving forces in this invention utilizes the energy created from a compressed fluid such as a canister of CO₂, from a disposable battery powered motor or pump, and/or springs. The energy used may be sufficient to generate enough force to withstand a pressure range of −14.7 to 30 psi. The energy source may also be able to supply the forces within milliseconds of activation and be able to supply the desired forces repeatability with minimal delay between previous supply of force. The energy source may also be able to have enough power to supply the forces through a given medical procedure. For example, a DVT thrombectomy procedure in which a physician removes clot from the venous system may, in a worst case, have a clot burden caused by an IVC filter where the majority of the Internal Vena Cava (IVC), and the entire venous track from iliac to popliteal is occluded in both legs. In this instance, the amount of clot to be removed may be the volume of the vasculature. This could be ˜150-cc of clot based on the anatomical study conducted by Dr. Santilli et al., as described in “Superficial Femoral popliteal vein: An Anatomic Study” (2000), which is herein incorporated by reference in its entirety. It is also desired that the energy source can move the aspirator piston at a speed to maximize the flowrate within the given aspiration catheter that the fluidic driven is attached to. Maximizing the flowrate at least momentarily such that the aspiration flowrate through the catheter is faster than the blood flow within the vessel causes a reversal of blood flow which can pull downstream obstructive material towards the aspiration orifice of the catheter and/or at least partially through the aspiration lumen. Examples of a desired aspiration flowrates are ≥60 cc/sec through an aspiration lumen having a cross-sectional area of 0.159 cm2 and ≥150 cc/sec through an aspiration lumen having a cross-sectional area of 0.323 cm2. Using an aspirator piston with a diameter of 2.5 cm, the piston velocity would have to be ≥12 cm/sec and force applied would need to be ≥15 lbs. to overcome the dynamic and static forces of the syringe. Pressurized fluid such as CO2 is an ideal source of energy because it is packaged in the liquid state which creates a relatively small form factor for a large volume of gas and can achieve high pressures due to the phase change of the fluid to gas. An example of the form factor is a 25-gram CO2 canister is only 2.5 cm in diameter and 11 cm long and contains 13,500-cc of gas which is 4700 Joules of energy.

FIGS. 11-13 illustrate examples of the apparatuses described herein that utilize compressed gas and/or springs to drive the aspirator piston to aspirate fluid and obstructive material from a body and returns the fluid back to the body. For example, FIG. 11 illustrates a fully disposable fluidically driven aspirator system for clot extraction that requires only a compressed canister and a battery. This embodiment, referred to herein as a manual disposable fluidic driven aspirator, may be a fully disposable fluidic driven aspirator that contains the same features as described above but this embodiment can be sterilized and be used completely in a sterile field and thrown away at the end of the procedure. This may be desired for emergent procedures that don't have time to a complex setup time. In some examples the apparatus utilizes pressurized fluid, such as from a compressed air or CO2 canister, that are opened at time of use. In some examples the embodiment will be battery powered, however it should be understood that battery power is not required. The canister can be opened by the user at the start of the procedure by inserting the fluid canister into the compressed fluid chamber and puncturing the canister on the canister puncture needle, allowing the fluid within the canister to convert to gas and pressurize the system. The system may then control the delivery and dosing of the compressed gas to enable the user or the controller of the system to apply vacuum or positive pressure to the aspiration lumen of the catheter that is attached the aspirator port of the aspiration device. This embodiment comprises of the following: compressed fluid chamber, canister puncture needle, expansion reservoir, a regulator, at least one control valve, fluid control lines (FCL, FCL′), driver cylinder, driver piston, driver fluid port 1, driver fluid port 2, drive shaft, aspirator piston, aspirator cylinder, an aspirator inlet port, and an aspirator outlet port. Also shown in this embodiment, the aspiration inlet port is fluidically connected to the aspiration lumen of an aspiration catheter and the aspiration outlet port is fluidically connected to a blood and clot management system. Additionally in this embodiment, the blood and clot management system is fluidically connected to a return catheter. In use, the user inserts the fluid canister into the compressed fluid chamber. The user may then tighten the top of the compressed fluid chamber to the bottom of the compressed fluid chamber enclosing the fluid canister and force the puncture needle into the bladder of the fluid canister. In some embodiments, the fluid canister nipple is threaded so the compressed fluid chamber would only have a bottom where the canister puncture needle is positioned and would have female threads that match the threads on the fluid canister nipple. Once the fluid canister is punctured, the compressed fluid flows out of the canister through a tube into the expansion reservoir and into the regulator all while the fluid is converting to gas and pressurizing the system. The compressed gas then passes through the regulator until a desired pressure is reached between the regulator, the valve, the FCL′, and the driver cylinder volume connected to driver fluid port 2. When the valve is activated, the pressurized gas is allowed to vent out of driver fluid port 2 and pressurized gas fills the volume of the driver cylinder that is connected to driver fluid port 1. The fluid entering driver fluid port 1 drives the driver piston, driver shaft, and aspirator piston in the first direction. The rate at which the pistons move is controlled by the pressure and flowrate of the gas into driver fluid port 1 and the rate at which the gas vents out of driver fluid port 2. In some embodiments, the pressure of the gas entering the driver fluid port 1 is maintained greater than 30-psi. This action generates a vacuum pressure between the aspirator piston and the aspiration orifice causing the aspiration inlet port to open and the aspiration outlet port to close and for fluid and obstructive material proximate to the aspiration orifice to enter into the Aspiration Lumen when aspiration catheter is inside the body. When the valve is released or deactivated, the control valve internals shift and the gas vents out of driver fluid port 1 and gas fills the volume of the Driver Cylinder that is connected to Driver Fluid Port 2. This action creates a positive pressure within the Aspiration Cylinder that closes the Aspiration Inlet port, opens Aspiration Outlet Port, and pushes the fluid and obstructive material out of the Aspiration cylinder into the Clot Chamber. Using the same positive pressure the obstructive material is strained from the fluid and the fluid is pushed through a filter media, the one-way Blood Valve and into the Blood Chamber. The fluid enters the Blood Chamber above the BC Outlet through the BC Inlet filling the Blood Chamber. As the Blood Chamber fills, the Fluid continues into the Return Catheter and back into the Body using the positive pressure from the Fluidic Driven Aspirator.

FIG. 12 illustrates another fully disposable fluidically driven aspirator that has the ability to activate the pressure source autonomously based on at least one measured parameter from the procedure. This embodiment comprises of the same fluidic construction as FIG. 11 except for the Control Valve is a battery powered 5/2 solenoid valve and the power to the solenoid valve for actuation is controlled and monitored by a Controller that is electrically connected to Sensors on the Aspiration Catheter.

A functional prototype of this invention is shown in FIG. 13. This embodiment comprises of a Compressed Fluid Chamber made of aluminum with female threads concentrically around the Canister Puncture Needle. The Compressed Fluid Chamber also has an on/off valve that controls the flow of the Compressed fluid. The Compressed Fluid Chamber also has a polymeric O-ring around the Canister Puncture Needle to aid in sealing. The Compressed Fluid Chamber has then connected to a Regulator with a Gauge with a small volume of tubing that allows for fluid to convert to gas prior to the Regulator. The Regulator is then connected to a 24-volt 5/2 solenoid valve. The Solenoid is then electrically connected to a Button and 3 9-volt batteries in series. The solenoid is then pneumatic connected to the Driver Cylinder, air cylinder, and the exhaust ports of the solenoid are restricted to achieve desired stroke rate on of the Driver Piston. The Driver Fluid Port 1 vent is restricted more than the Driver Fluid Port 2 vent to have the positive pressure stroke rate be slower than the negative pressure stroke. The Driver Cylinder and Aspiration Cylinder are positioned side by side. The Aspiration Cylinder is constructed of a rigid translucent material such as polycarbonate. The Drive Shaft connecting the Driver Piston and the Aspirator Piston is made of a rigid plastic such as Nylon but could be made of metal. The Aspiration Cylinder has an inner diameter of 2.5 cm and has a total volume of 15-cc. On the Distal end of the Aspiration Cylinder, the Aspiration Inlet and Outlet Port Assemblies is affixed creating an airtight seal between the Cylinder and Port assembly. In the Port Assembly, there are two one-way duckbill valves made of silicone positioned to allow fluid in one Port while closing the second Port when the Aspiration Cylinder moves in the first direction and then the Port positions change when the Aspiration Cylinder moves in the second direction. Attached to the Ports are silicone tubes that are placed in a bucket of water. The prototypes regulator was set to 40-50 psi. The canister is screwed into the Compressed Fluid Chamber until the canister was punctured. The valve was then actuated until pressure in the regulator went below 30 psi.

FIG. 14 shows a graph showing the volume of fluid, blood, that can be moved in and out of the Aspiration Cylinder using Compressed Gas.

FIGS. 15 and 16 illustrate alternative fluidic driver constructions which have one fluidic drive compartment and one spring return compartment. Instead of applying positive and/or negative pressure on both sides of a fluidic drive piston, as previously describe, fluidic driver embodiments can utilize a coil or other compression/extension spring placed in one fluidic compartment to provide or enhance a return displacement of the fluidic driver piston. In such embodiments, the fluidic drive piston can be initially displaced in one direction by pressurization of a first compartment and returned in the opposite direction by the spring when pressurization id stopped or decreased.

Disposable Fluidically Driven Aspirator for Clot Extraction from a Vessel

The minimally invasive removal of unwanted material from the body is done all over the body. In most of the procedures where unwanted material is removed there is a need for preserving the fluid that is also within that part of the body. An example of this is thrombectomy. Thrombectomy is the removal of thrombus (blood clots) from various parts of the human vasculature. The current state of the art in thrombectomy includes several types of systems, including manual aspiration with a syringe, aspiration via vacuum-pump with and without computerized valve control and/or with and without clot maceration, and physical scraping/catching of clot from the vessel and vessel walls. The current systems have shown that mechanically removing of clot either via aspiration or physical force is effective; however, there are safety concerns, and the systems are inefficient from a blood loss, user cognitive and physical exertion, and procedure time.

Systems which include a vacuum chamber and a vacuum-valve to connect the aspiration lumen to the vacuum chamber suffer from hemolysis of the blood, which renders it unreturnable to the patient. This occurs when the blood is exposed to open vacuum/air for extended periods, which also causes the blood to foam and degas. Procedures with these types of systems tend to incur excessive blood loss and may have to prematurely stop the procedure due to blood loss concerns. These systems also require a capital piece of equipment which makes setup more difficult and removes controls from the user in the sterile field as those controls must be placed out of the sterile field.

Systems which require manual operation for clot extraction are ill-suited to peripheral thrombectomy procedures such as deep-vein thrombosis (DVT) because the clot burden needed to be removed could be quite large. Using these systems require many repetitive steps to sufficiently remove the clot burden. This includes both manual aspiration systems as well as systems which physically pull the clot from the vessel. For manual aspiration systems though burdensome due to the repetitive steps, the aspiration syringe design does achieve the highest pressure differentials while reducing the time the blood is exposed to the vacuum pressure which enables effective clot burden removal while maintaining the quality of the blood to allow the blood to be returned.

This invention addresses these shortcomings and describes an improved way for generating negative pressure and positive pressure within a cylinder for the movement of fluid and unwanted material from the body and/or into a collection chamber. In some embodiments of the invention, the fluid removed is filtered and able to be returned back to the body. In further embodiments, the filter fluid is returned directly back to the body using the positive pressure generated by the invention. The improvements of this invention include: reduced procedure time, reduced number of required operators, and user effort to generate negative pressure by eliminating the need to connect and disconnect the pressure source or having to exert a force on the aspiration source, such as pulling and pushing on a syringe; eliminates the need for capital equipment; auto activation of pressure source based on procedure feedback; enables on-demand user activation of pressure source that resides all in the sterile field; increased control of the whole system pressure source and puts the controls in the sterile field to enable the user easy access; eliminates procedure steps when moving fluid and material from and to the body; provides features that can sense and determine what is at the aspiration opening of the aspiration catheter; and/or provides sensing for monitoring for air and clot within the return fluid going back to the patient.

These improvements are achieved by using mechanical forces to move an aspirator piston within a cylinder at a rate to generate a pressure differential that can adequately move fluid and unwanted material. The invention is designed to fluidly connect to an aspiration lumen of a catheter. This invention has the ability to control the rate at which the system generates a pressure differential between the aspirator piston face and the distal aspiration opening of an aspiration lumen at least greater than 10 mmHg and preferably greater than 600 mmHg as well as controlling the rate of the aspirator piston forward. The driving forces in this invention utilizes the energy created from a compressed fluid such as a canister of CO₂, from a disposable battery powered motor or pump, and/or springs. It is important that the energy used is sufficient to generate enough force to withstand a pressure range of −14.7 to 30 psi. The energy source needs to also be able to supply the forces within milliseconds of activation and be able to supply the desired forces repeatability with minimal delay between previous supply of force. The energy source needs to also be able to have enough power to supply the forces through a given medical procedure. For example, a DVT thrombectomy procedure is where a physician removes clot from the venous system. In a worst case, the clot burden could be caused by an IVC filter where the majority of the Internal Vena Cava (IVC), and the entire venous track from iliac to popliteal is occluded in both legs. In this instance, the amount of clot to be removed would be the volume of the vasculature. This would be ˜150-cc of clot (e.g., based on the anatomical study conducted by Dr. Santilli et al., in their Superficial Femoral popliteal vein: An Anatomic Study published in 2000, herein incorporated by reference). It is also desired that the energy source can move the aspirator piston at a speed to maximize the flowrate within the given aspiration catheter that the fluidic driven is attached to. Maximizing the flowrate at least momentarily such that the aspiration flowrate through the catheter is faster than the blood flow within the vessel causes a reversal of blood flow which can pull downstream obstructive material towards the aspiration orifice of the catheter and/or at least partially through the aspiration lumen. Examples of a desired aspiration flowrates are ≥60 cc/sec through an aspiration lumen having a cross-sectional area of 0.159 cm2 and ≥150 cc/sec through an aspiration lumen having a cross-sectional area of 0.323 cm2. Using an aspirator piston with a diameter of 2.5 cm, the piston velocity would have to be ≥12 cm/sec and force applied would need to be ≥15 lbs. to overcome the dynamic and static forces of the syringe. Pressurized fluid such as CO₂ is an ideal source of energy because it is packaged in the liquid state which creates a relatively small form factor for a large volume of gas and can achieve high pressures due to the phase change of the fluid to gas. An example of the form factor is a 25-gram CO₂ canister is only 2.5 cm in diameter and 11 cm long and contains 13,500-cc of gas which is 4700 Joules of energy.

FIGS. 11 to 13 illustrate embodiments of the present invention that utilizes compressed gas and/or springs to drive the aspirator piston to aspirate fluid and obstructive material from a body and returns the fluid back to the body.

FIG. 11 illustrates a fully disposable fluidically driven aspirator system for clot extraction that requires only a compressed canister and a battery. This embodiment, the manual disposable fluidic driven aspirator, is a fully disposable fluidic driven aspirator that contains the same features as described above previous embodiments, but this embodiment can be sterilized and be used completely in a sterile field and thrown away at the end of the procedure. This is very desirable for emergent procedures that don't have time to a complex setup time. The current embodiment utilizes pressurized fluid, such as compressed air or CO₂ canister, that are opened at time of use. In configurations, the embodiment will be battery powered but battery power isn't required. The canister can be opened by the user at the start of the procedure by inserting the fluid canister into the compressed fluid chamber and puncturing the canister on the canister puncture needle allowing the fluid within the canister to convert to gas and pressurize the system. the system then has means of controlling the delivery and dosing of the compressed gas to enable the user or the controller of the system to apply vacuum or positive pressure to the aspiration lumen of the catheter that is attached the aspirator port of the aspiration device. This embodiment comprises of the following: compressed fluid chamber, canister puncture needle, expansion reservoir, a regulator, at least one control valve, fluid control lines (FCL, FCL′). Driver cylinder, driver piston, driver fluid port 1, driver fluid port 2, drive shaft, aspirator piston, aspirator cylinder, an aspirator inlet port, and an aspirator outlet port. Also shown in this embodiment, the aspiration inlet port is fluidically connected to the aspiration lumen of an aspiration catheter and the aspiration outlet port is fluidically connected to a blood and clot management system. Additionally in this embodiment, the blood and clot management system is fluidically connected to a return catheter. in use, the user inserts the fluid canister into the compressed fluid chamber. The user would then tighten the top of the compressed fluid chamber to the bottom of the compressed fluid chamber enclosing the fluid canister and forcing the puncture needle into the bladder of the fluid canister. in some embodiments, the fluid canister nipple is threaded so the compressed fluid chamber would only have a bottom where the canister puncture needle is positioned and would have female threads that match the threads on the fluid canister nipple. Once the fluid canister is punctured, the compressed fluid flows out of the canister through a tube into the expansion reservoir and into the regulator all while the fluid is converting to gas and pressurizing the system. The compressed gas then passes through the regulator until a desired pressure is reached between the regulator, the valve, the FCL′, and the driver cylinder volume connected to driver fluid port 2. When the valve is activated, the pressurized gas is allowed to vent out of driver fluid port 2 and pressurized gas fills the volume of the driver cylinder that is connected to driver fluid port 1. The fluid entering driver fluid port 1 drives the driver piston, driver shaft, and aspirator piston in the first direction. The rate at which the pistons move is controlled by the pressure and flowrate of the gas into driver fluid port 1 and the rate at which the gas vents out of driver fluid port 2. In some embodiments, the pressure of the gas entering the driver fluid port 1 is maintained greater than 30-psi. This action generates a vacuum pressure between the aspirator piston and the aspiration orifice causing the aspiration inlet port to open and the aspiration outlet port to close and for fluid and obstructive material proximate to the aspiration orifice to enter into the aspiration lumen when aspiration catheter is inside the body. When the valve is released or deactivated, the control valve internals shift and the gas vents out of driver fluid port 1 and gas fills the volume of the driver cylinder that is connected to driver fluid port 2. This action creates a positive pressure within the aspiration cylinder that closes the aspiration inlet port, opens aspiration outlet port, and pushes the fluid and obstructive material out of the aspiration cylinder into the clot chamber. Using the same positive pressure the obstructive material is strained from the fluid and the fluid is pushed through a filter media, the one-way blood valve and into the blood chamber. The fluid enters the blood chamber above the BC outlet through the BC inlet filling the blood chamber. As the blood chamber fills, the fluid continues into the return catheter and back into the body using the positive pressure from the fluidic driven aspirator.

FIG. 12 illustrates another fully disposable fluidically driven aspirator that has the ability to activate the pressure source autonomously based on at least one measured parameter from the procedure. This embodiment comprises of the same fluidic construction as FIG. 11 except for the control valve is a battery powered 5/2 solenoid valve and the power to the solenoid valve for actuation is controlled and monitored by a controller that is electrically connected to sensors on the aspiration catheter.

As shown in FIG. 17, a secondary pressure source may be added to the system of FIG. 12 to generate a positive pressure greater than that venous pressure and the pressure resistance needed to be overcome to reinfuse the filtered blood back to the patient. The positive pressures needed to be generated is between 10-1300 mmHg (0.2-25 psi) to generate blood return flowrates greater than 0.2 cc/sec. In some embodiments the pressure source and system resistance would generate a flowrate 0.5-30 cc/sec. The pressure source can be incorporated anymore within the blood return fluid path. In some in embodiments, the pressure source would be positioned between the blood chamber and the return catheter. Pressure source could be a peristaltic pump (roller pump) or a manual syringe having a two one-valve manifold or a stopcock that allowed the user to manually pull blood from the blood chamber and push the blood from the syringe barrel into the patient. This optional secondary pressure source would allow the returning of the blood to be done independent to the aspiration function of the powered system to ensure blood return doesn't impede the procedure time. This secondary pressure source also allows the physician to run the blood return independent of the thrombectomy procedure and/or have another clinician operate and maintain that portion of the system.

As also shown in FIG. 17, an additional filter (Filter 2) can be placed between the blood chamber and the return catheter to ensure blood coming from the blood return fluid path is filtered just before being returned to patient. This is important to ensure blood is filtered right before it enters back to the patient. This would prevent if blood outside of the body starts to clot. This filter 2 will have filtering media having a porosity of 30-200 micron.

As shown in FIG. 18, FCL′ and FCL could have 2 independent regulators (regulator 1 & regulator 2) to enable the driver fluidic port 2 to have a different pressure than the pressure set for driver fluid driver 1. This allows for tailoring the backward motion and forward motion speed of the aspiration piston to generate different flowrates for blood coming into the syringe barrel (aspiration flowrate) and for blood being pushed out of the syringe barrel (blood return flowrate). This flowrate can also be varied by throttling the exhaust flowrate of the other side of the driver cylinder or control valve via a flow restrictor. In preferred embodiments, regulator for the aspiration flowrate will be set higher than the pressure of the regulator for blood return. In preferred embodiments, the aspiration flowrate will be greater than blood return flowrate.

A functional prototype of this invention is shown in FIG. 13. This embodiment comprises of a compressed fluid chamber made of aluminum with female threads concentrically around the canister puncture needle. The compressed fluid chamber also has an on/off valve that controls the flow of the compressed fluid. The compressed fluid chamber also has a polymeric o-ring around the canister puncture needle to aid in sealing. The compressed fluid chamber was then connected to a regulator with a gauge with a small volume of tubing that allows for fluid to convert to gas prior to the regulator. The regulator is then connected to a 24-volt 5/2 solenoid valve. The solenoid is then electrically connected to a button and 3 9-volt batteries in series. The solenoid is then pneumatic connected to the driver cylinder, air cylinder, and the exhaust ports of the solenoid are restricted to achieve desired stroke rate on of the driver piston. The driver fluid port 1 vent is restricted more than the driver fluid port 2 vent to have the positive pressure stroke rate be slower than the negative pressure stroke. The driver cylinder and aspiration cylinder are positioned side by side. The aspiration cylinder is constructed of a rigid translucent material such as polycarbonate. The drive shaft connecting the driver piston and the aspirator piston is made of a rigid plastic such as Nylon but could be made of metal. The aspiration cylinder has an inner diameter of 2.5 cm and has a total volume of 15-cc. On the distal end of the aspiration cylinder, the aspiration inlet and outlet port assemblies is affixed creating an airtight seal between the cylinder and port assembly. In the port assembly, there are two one-way duckbill valves made of silicone positioned to allow fluid in one port while closing the second port when the aspiration cylinder moves in the first direction and then the port positions change when the aspiration cylinder moves in the second direction. Attached to the ports are silicone tubes that are placed in a bucket of water. The prototypes regulator was set to 40-50 psi. The canister is screwed into the compressed fluid chamber until the canister was punctured. The valve was then actuated until the pressure in the regulator went below 30 psi.

FIG. 14 shows a graph showing the volume of fluid, blood, that can be moved in and out of the aspiration cylinder using compressed gas.

### Plunger Position Control Algorithms:

The section above described a very simple plunger position control algorithm, which simply pulled backwards whenever clot was actively being detected at the tip, stopped or pushed forwards whenever clot was not being detected at the tip, and pushed all the way to the very front if the syringe plunger touched the back of the syringe. Other more sophisticated control algorithm options are described here.

The key aspects to consider when devising a plunger position control algorithm include not only handling the endpoints of the syringe travel, but also the likelihood of causing vessel collapse, or causing the aspiration orifice to latch onto the vessel wall, and the desire to break up and extract bites of the clot for improved aspiration effectiveness.

To prevent vessel-collapse during aspiration, the system may be configured to prevent removal of more fluid volume than can be locally replaced by the body. Long time intervals of full-vacuum aspirations can easily cause vessel collapse and system lock-up. To prevent this, either the system can implement slow-flow-rate controlled plunger pulls or periodic fast-pull intervals with recovery time between them as part of a prescribed duty cycle. Either way the intent is to meter the overall extraction flow and prevent more volume extraction than the rate at which the body is able to locally replace that fluid in the area of the catheter tip—or if the extraction rate is higher, it is only slightly higher. Ideally the vessel can locally constrict as clot and blood are extracted through the aspiration lumen, but not to the point of vessel collapse and lock-up.

The same argument about flow rates above applies for preventing or reducing the chances of vessel-wall latch with the aspiration orifice. Slower extraction flow rates, or a duty cycle of applied full-flow-rate sips between which the plunger is no longer being actively pulled backwards, are both capable of reducing the propensity of the system to latch onto the vessel wall. It has been found that full-vacuum rate syringe pulls in a 16F catheter that last for >500 ms are much more likely to latch onto the vessel wall. Sip durations of 50 ms-200 ms are best for wall-latch prevention as well as vessel-collapse prevention.

The desire to also use aspiration power to help break apart the clot into manageable size chunks for easier aspiration leads one to preferentially use a method of duty-cycled full-speed plunger pulls with recovery time, rather than a slow-rate pull method. This also helps ensure the clot engages with the aspiration orifice rim with the most force, helping to rip it apart for improved aspiration efficiency.

Example diagrams showing how plunger position and speed can be prescribed to help prevent vessel collapse, prevent vessel-wall latch, and help break up clot into manageable pieces for aspiration are shown in FIGS. 19-22, which shows alternative plunger control algorithm timing diagrams that may be used to prevent vessel collapse and vessel wall latch. The duty-cycled algorithm (dotted line) also has the added benefit of ripping chunks of clot off at a time for improved aspiration efficiency. FIG. 20 (top panel) shows sensed tip condition as a function of time. Clot detection is indicated by the boxes. FIG. 20 (bottom panel) is a graph showing plunger position (distance from syringe front, from front to back) on the Y-axis as a function of time. The solid line shows Use controlled slow-rate flow plunger pulls. The dotted line shows Use fast-rate with duty cycle of recovery time. (a) shows a region of the graph with a steep slope (fast speed) to match or exceed max aspiration lumen flow rate. (b) shows a region of the graph with no active pull and recovery time for body fluid to refill vessel area near tip. (c) shows a gradual slope: a slow speed (to approximate body refill flow rate near tip. (d) and (c) show forward motion slope: speed to push blood/clot to blood return filter system (rate dependent on return system pressure).

One aspect not described above is that back of the syringe location may be used as a stop for an implementation that uses fast-pull motion in a duty-cycle fashion. The entire syringe stroke-length may be utilized in a fast-frequency motion to achieve the above-mentioned benefits. That way precise position control within mid-stroke of the syringe is not required. This is shown in FIG. 21. FIG. 21 (top panel) shows sensed tip condition as a function of time. Clot detection is indicated by the boxes. FIG. 21 (bottom panel) is a graph showing plunger position (distance from syringe front, from front to back) on the Y-axis as a function of time. The solid line shows Slope of max flow rate through aspiration lumen under 1 atm of vacuum. The dotted line shows small syringe using full-stroke at fast rate. (a) shows a single cycle of small syringe. (b) points to a positive slope on the graph, positive slopes steeper than this pull vacuum in the syringe faster than the blood can fill it through the aspiration lumen). Also note that there is an implied slope in these graphs which is the max flow rate dictated by the aspiration lumen size and the latm of vacuum. Any motion faster than this will result in vacuum appearing for some time during the motion of the plunger.

Another aspect of plunger position control algorithms may include the intentional additional aspiration time which can continue after the end of clot-sensing at the tip of the catheter. This is shown in FIG. 22 below. FIG. 22 (top panel) shows sensed tip condition as a function of time. Clot detection is indicated by the boxes. FIG. 22 (bottom panel) is a graph plunger position (distance from syringe front, from front to back) on the Y-axis as a function of time. The dotted line shows Syringe using Full-stroke for aspiration. (a) shows additional suction time interval to pull additional blood into the aspiration orifice at the tip after the clot has cleared the tip. In FIG. 22, the plunger continues to aspirate after the completion of sensing the clot at the tip of the catheter. This may help lubricate the catheter and can clear the shaft of known clot material. Once clot has passed the aspiration orifice and is in the aspiration lumen, the system could simply stop aspiration right away to minimize the amount of aspirated blood. However, this creates an aspiration lumen nearly full of just clot material, which can be harder to move freely down the shaft as the friction and momentum can result in a clogged shaft. One way to improve the performance is the addition of time after the clot has finished entering the aspiration orifice, to pull additional blood into the shaft to help with overall lubrication. The algorithm may ensure that the amount of blood pulled after the clot has cleared the tip would be enough to fill the aspiration lumen and ensure a clear catheter. This allows for future steps such as insertion of accessories or pushing contrast down the lumen for visualization.

Timing Aspiration with Cardiac Cycle

Any of the methods and apparatuses described herein may be configured to coordinate aspiration (or in some cases injection of fluid) with the patient's cardiac cycle (e.g., diastole/systole). For example, to optimize aspiration performance, it is best if there is sufficient blood to replace the volume of blood and clot being aspirated at the catheter tip. Due to the patient's cardiac cycle, there may be variations in the blood pressure and nearby available blood in the local vessels during a thrombectomy procedure. Any of these methods and apparatuses may synchronize the aspiration with the cardiac cycle of the patient. For example, any of these apparatuses may include one or more inputs for a sensor sensing a cardiac indicator (e.g., blood pressure, pulse, ECG, etc.). The system, e.g., the controller, can be configured to help avoid vessel collapse and vessel wall latching based on the input cardiac indicator(s). In some examples the aspiration may be applied when the pressure is highest and/or when the pressure is above a threshold (relative to the patient blood pressure) and thus, when there is likely to be available blood around the catheter tip to replace the aspirated contents.

In general, any of these systems can determine the current portion of the cardiac cycle the patient is currently in. For example, any of these apparatuses may be configured to use electrical inputs such as an electrocardiogram (EKG) which is well understood; for example, the system may detect a QRS wave to see whether the patient is before or after the ventricular contraction. Another technique to synchronize the apparatus with the cardiac cycle may include using pressure measurements. The apparatus may use artifacts in the pressure measurements at the catheter to determine the optimal timing to perform an aspiration.

Once the timing with respect the cardiac cycle is known, the system could either be fully-automatic and perform aspirations at the optimal time when clot is sensed at the catheter tip, or it could be semi-automatic and the user could press a button to indicate aspiration is desired, and then the system could wait until the opportune moment in the cardiac cycle to actually perform the aspiration. Thus, the controller may include control logic to apply this technique.

### Additional Syringe Arrangements

A single syringe system as described above may be limited by the fact that whenever the syringe plunger is being pushed forward, the system cannot simultaneously be aspirating clot even if the information from the sensing elements (sensors) would instruct the system otherwise. This non-functional state of the system may be communicated to the user, e.g., if it lasts more than a couple of seconds. To help optimize the system for continuous performance, a multi-syringe arrangement may be implemented which allows for at least one syringe to be always available for vacuum application. This may be achieved using a fluid manifold connection scheme as shown in FIG. 24. FIG. 24 schematically illustrates a multi-syringe arrangement that may allow for continuous vacuum capability.

In the layout in FIG. 24, there could be multiple syringes of different volumes or form-factor sizes. Having a smaller barrel diameter on a syringe is known to decrease the force required to displace that plunger a given distance, therefore allowing operation at higher rates for a same given force (whether it is driven from a motor, pneumatic, or other energy source). The advantage with a dual syringe layout is then increased with the dual sizes, having one for quicker and smaller volume displacements and a larger one for longer sustained vacuum pulls. The relative sizes of these syringes could be in the 1 cc to 80 cc size ranges. The sensing algorithm along with pressure measurements could relay sufficient information to the software to decide and control which state of the plungers should be in, and ultimately which and when to pull back. One could be used as a primary and when the primary is in a state that cannot deliver vacuum, then the other one could be sized to just provide enough vacuum while the other is in the downtime of forward advancement. Alternatively, multiple syringes could be used in parallel to ‘stack’ vacuum on top of each other as clot is being pulled through the aspiration lumen-such as when it is clogged. Once one syringe vacuum is pulled and the pressure monitoring shows there is a clog, the second syringe may be used to help increase the vacuum and maintain it. In general, the speed and position of each of the multiple syringes may be adjusted based on the live sensing data coming from the tip as well as the pressure sensors.

The concept of multiple syringes may be extended to a larger number of syringes, each smaller volume, where the system can operate like an engine with several pistons. In this way, each “sip size” may be quite small and the system is capable of nearly continuous vacuum pull as required, while still maintaining the blood return capability.

A “two-syringe” blood filtering and return system 10 for aspirating a mixture of clot and blood from a patient, filtering clot from the blood, and returning the filtered blood to the patient is shown in FIG. 24. The system 10 includes a first blood pump 12, typically a syringe, and a second blood pump 14, typically also a syringe. The first blood pump 12 is configured to draw the mixture of blood and clot from an aspiration catheter 20 having a distal port 22 dispose proximate a region of clot C in a patient blood vessel BV. More specifically, the first blood pump draws the mixture of clot and blood through a three-way connector 18 into a receptacle 12a, such as a syringe barrel, by retracting a plunger 12b in a conventional manner. The mixture of clot and blood is then delivered into and through filter assembly 16 to form separate volumes of filtered blood and separated clot within the filter assembly. The three-way connector 18 may be configured as two one-way valve which direct inflow from the aspiration catheter to the receptacle 12a and outflow from the receptacle 12a to the filter assembly 16, as indicated by the arrows on the three-way connector 18.

As stated above, flow of the clot and blood mixture induced by the first blood pump 12 causes the mixture to separate into a clot fraction and a filtered blood fraction in the filter assembly 16. Details on a suitable filter assembly are described herein below.

Once separated into clot and filtered blood fractions within the filter assembly 16, the second blood pump 14, shown as a syringe, can be used to return filtered blood to the patient in any one of at least two ways. In a first protocol, the clot and blood mixture is delivered to the filter assembly 16 with sufficient pressure to cause the filtered blood to enter a receptacle 14a of the second blood pump 14 typically a barrel of a syringe pump. A plunger 14b of the second blood pump 14 can then be advanced to deliver the filtered blood directly back to the patient through ta filter return tube 46 which may be connected to a blood return sheath, for example a sheath used to introduce the aspiration catheter 20 to the patient. Alternatively, once filled with syringe barrel 14a or other filtered blood receptacle is at least partially filled, the second syringe blood pump 14 may be detached from the filter 16 and used to return the filtered blood elsewhere to the patient, for example to a sheath or cannula introduced to the patient's venous vasculature.

### Vacuum Release and Burping Options

The system may sometimes become latched to a vessel wall and require vacuum-release. For this condition, there are several ways to release vacuum or otherwise “burp” the system. The simplest is a mechanical valve the user can use to let air in the system and release pressure that way into the aspiration lumen. That mechanical valve can also be electro-mechanical, and system controlled as part of a de-latch algorithm. Alternatively, rather than air used for re-pressurizing the aspiration lumen, fluid can be used. This could either be saline (such as a saline bag), or preferably the patient's own blood could be used. The blood return line could be split, and one line fed back to provide the small amount of volume required to re-pressurize the aspiration lumen when required. An example of this is illustrated schematically in FIG. 25. A pressure release (burping) valve may be positioned anywhere in the system, including before the pump, in the blood line, at the blood filtering device, etc.

In some circumstances, it is useful to re-pressurize the lumen of an aspiration catheter when a vacuum is no longer deemed useful to the clinical procedure, including when the catheter becomes latched onto a vessel wall. For example, the user may release the vacuum in the system to allow the catheter to move to another location. In any of these methods and apparatuses, vacuum may be released by allowing other liquid to enter the vacuum cavity, rather than air, because if air is allowed into the catheter, subsequent aspirations will first need to extract the air before full vacuum can be achieved. Liquid added may be saline (e.g., from an external reservoir, and/or blood, e.g., from another portion of the blood circuit. For example, FIG. 25 illustrates how blood from the blood return line could be used as the liquid to fill that vacuum. This, however, is not necessarily ideal since then some blood could be circulating within the system outside the body for a while and can expose that blood to more vacuum than desired which can lead to hemolysis. Alternatively, saline (or other compatible physiological solution) may be used, e.g., introduced at one or more locations within the circuit, such as at a saline port 981, 982 between the pump and the aspiration catheter either before 981 and/or after 982 the valve directing fluid into the blood return/filtering line.

For example, a reservoir (e.g., syringe) filled with saline may be coupled to the port 981 at the catheter handle and a valve on the port opened to allow the syringe (or other pressurized reservoir) to fill the vacuum void in the catheter shaft, handle, and system with saline. This may allow the user to release vacuum without introducing air, and the catheter can then be moved freely.

In any of these methods and apparatuses, the system may include a small saline reservoir, and releasing the vacuum using saline from the reservoir may be integrated into the system. This release of vacuum may be manual, automatic or semi-automatic. In some examples the user could manually operate a valve to allow saline from the reservoir to enter the vacuum of the aspiration catheter and system syringe. This eliminates the need to attach a pre-filled syringe. Any of these apparatuses or methods may include a valve between a saline reservoir and a system vacuum such that saline could fill the space but then be shut off for normal operation to allow for aspirations. In some cases the release may be triggered by a user control (e.g., button, switch, etc.) and/or an automatic control, upon sensing “locking” onto the wall at the aspiration opening of the aspiration catheter, e.g., using one or more sensors (e.g., impedance sensors, optical sensors, etc.) at the distal end of the aspiration catheter, and/or pressure sensors sensing pressure within the aspiration lumen.

For example, of these methods and apparatuses may include a power-assist in the release of vacuum such that the valve between the saline reservoir and the system vacuum is controlled by the system. A user could press a button to release the vacuum by delivering saline, and the system may automatically cycle the valve to allow saline to release the vacuum and then re-close for normal operation. The flow of saline into the system may be regulated, e.g., to avoid damage to the system (catheter), uncontrolled movement of the tip, and/or damage to blood cells. For example, the release of pressure may be gradual (extending over a few seconds, e.g., 1 second or more, 2 seconds or more, 3 seconds or more, 4 seconds or more, 5 seconds or more, etc.). Alternatively the release may be fast (e.g., less than 1 second, less than 0.75 seconds, less than 0.5 seconds, etc.).

As mentioned, in some cases the system may automatically control the vacuum release valve and allow saline to enter the vacuum chamber when the system detects (e.g., through one or more sensors) that the tip of the catheter is currently latched onto a vessel wall. The sensing of the system and the knowledge of the condition at the catheter tip can inform the system to perform its own vacuum release to increase procedure efficiency.

### Injection of Agent

In addition to the injection of saline (or other biocompatible fluid) to release vacuum, in some cases these methods and apparatuses may be configured to inject an agent, such as contrast and/or a therapeutic material (drug, enzyme, etc.) into the system, including out of the aspiration catheter. For example, any of these methods and apparatuses may be configured to inject a fluoroscopic contrast solution into the catheter. In some cases the same ports (see, e.g., FIG. 25, ports 981, 982) may be used. A syringe of material may be used, or in some cases these apparatuses may be configured so that the user does not need to physically connect a syringes with these fluids to the system which may be manually intensive and may introduce air. In some cases it may be preferable to include a valve coupled to the port to control flow through that port, to allow a power injector to provide these fluids (saline and/or contrast and/or therapeutic) into the system upon demand. For example, such a port may be connected to one or more reservoirs at the beginning of a case and during the procedure, the user could select the fluids to inject and perform injections from the power injector without manually operating a syringe. These reservoirs may be pressurized and/or otherwise powered to deliver fluid into the system when the valve is opened. A multi-way valve may be coupled between the reservoir and the port. For example, as shown schematically in FIG. 9 a port 981 may be at the proximal end of the catheter or between the catheter and the pump; any of the apparatuses described herein may include such a port.

For example, any of these apparatuses may include a power injector configured to provide fluids with positive pressure. In some cases this pressure may be a high pressure, so the system lumens may be configured to handle the high pressures of a power injector. Parts of the system such as the pump (e.g., syringe piston 153 in FIG. 9 or any of the aspiration devices described herein) may be configured to handle this pressure without moving. For example, any of these systems may be configured to detect or determine when an injection was about to occur and may ‘lock’ the fluidic drive of the aspiration device (e.g., in some cases a piston) to prevent it from moving during injection. For example, the control (switch, button, etc.) for injecting fluid may be configured to trigger a lock on the aspiration device coupled to the fluidic driver to prevent movement during injection, and release the lock when injection is complete. In some cases the system may be configured to be told explicitly by the user through an interface (e.g., button, switch, etc.). In some cases a sensor, e.g., a pressure sensor could be used to sense the incoming high-pressure and then automatically put the system into a mode ready for fluid injections, including in some cases locking the fluidic driver.

Thus, any of these apparatuses may include a valve that can be open/closed by the system between the system fluidics and the power injector port, to prevent aspirations from sucking any fluids from the power injector port 981 during use.

### Blood Filtering Devices

FIGS. 26 to 32, disclose a blood filtration and deaeration assembly which can be used with any of the previously described clot aspiration system as well as most if not all other currently available and yet to be developed clot aspiration and thrombectomy systems. The clot aspirated from a patient comprise blood with entrained clot, and it is necessary the clot be removed from the blood before the filtered blood can be returned to the patient. The disclosed blood filtration and deaeration assemblies are configured to filter (separate) the blood from the thrombus, and to return the filtered blood to the patient.

A blood filtration and return circuit may be controlled by means of an aspiration syringe which creates variable pressures allowing for thrombus and blood to be aspirated from the patient and moved through the system into a separation and filtering chamber and being returned to the patient. The circuit typically includes several separate functional components that may allow for the ingress of air into the circuit via intended or improper use of the system. The ingress of air to the circuit poses a significant risk to the patient if it is not properly removed prior to returning to the sheath.

To reduce or eliminate the risk of air remaining in a blood return system as it travels from the system to the patient, deaeration systems, apparatus, and methods are provided for separating, filtering, and removing air from the blood return system may be integrated directly to the thrombus filtration chamber allows for the aspiration syringe to continue driving the circuit with no risk of endangering the patient due to either proper or improper use of the system, as shown in FIGS. 26 and 27. The apparatus features two distinct valving mechanisms which work in conjunction to separate, filter, and remove air from the system. As illustrated in FIG. 27, a system herein can include one or more of the following steps. With the outlet closed, system is prepared by flushing with heparinized saline. Saline fills the bubble chamber, causing the float to rise. Once the float reaches the top of the bubble chamber, a seal is created between the float and the air vent, creating a closed system. The fluid is contained in the bubble chamber due to the one-way valve and the outlet being closed. Alternatively, the system may be prepped by connecting the outlet to the sheath and opened. This will allow blood to bleed back into the bubble chamber and fill it.

As illustrated in FIG. 28, in the case that air is introduced to the clot chamber, it will be pushed to the bubble chamber during a blood return cycle. The introduction of air into the bubble chamber decreases the volume of fluid in the bubble chamber. If a sufficient volume of fluid is displaced by air within the bubble chamber, the float will drop, unsealing the system. At this point, air can be continued to be introduced and will be vented out of the air vent at the top of the bubble chamber. No forward flow to the patient will occur at this time, as the pressure in the bubble chamber (Pa) is at atmospheric pressure, and less than the venous pressure (Pv). Therefore, there is no risk of pushing air bubbles to the patient. Forward flow to the patient can return once the fluid in the volume raises the float and seals the system. This can occur automatically, as the venous pressure (Pv) will backfill the bubble chamber. This creates a safer and more efficient procedure as this occurs automatically, and the user does not need to monitor the blood return as closely. Alternatively, the user may introduce more fluid through a normal aspiration cycle.

The deaeration apparatus features a filtered blood inlet which connects to an outlet of the clot filtration canister system through a duckbill or other one-way valve. An outlet of the deaeration chamber connects to a length of silicon tubing which connects to a 40-micron filter before ultimately connecting to the flush lumen of the introducer sheath in the patient. The apparatus mounts directly to the clot canister where the blood inlet to the bubble eliminator from the clot canister is ⅜-inch diameter and features a one-way valve to prevent bi-directional flow of blood or gas between the bubble eliminator and the clot canister. The bubble eliminator is constructed of a rigid plastic such as polycarbonate. The inlet is placed at the bottom of the clot canister so the fluid in the canister, when subject to pressure, will move through the one-way valve into the bubble eliminator from the clot canister. The inlet enters a cavity at the lowest point in the center of the bubble eliminator which is approximately 10 cc in volume including a vertical spout where the blood will travel upwards into the main body of the bubble eliminator. The spout features a plurality of orifices and a diffusing umbrella shield placed above the orifices to control the flow of blood into the bubble eliminator. The orifices are approximately 2 inches above the inlet valve.

The main body of the apparatus features a drafted, conical shape which is wider at the bottom and thinner at the top. The volume of the body of the bubble eliminator is approximately 100 ccs with this embodiment limiting the ratio of fluid to air ratio to optimize the dampening of the inlet positive pressure from the aspiration piston. The greater the volume of air is with respect to the volume of blood, controlled by the volume of the bubble eliminator and the geometry of the float, the lesser pressures are generated allowing the seal to engage at different imposed forces. Above the umbrella shield there is a buoyant float which rests between guide rails. The float has an elongated neck that extends vertically from its base to create additional space between the sealing element at the top and the fluid which surrounds it. The float is approximately 2 inches tall, featuring a flat bottom and a thin, elongated neck which holds the sealing membrane at the top. The volume of space above the fluid line enhanced by the geometry of the float creates an air cushion of compressible gas which allows for the float to disengage more quickly as new infusions of blood and gas are pushed into the system. As a bolus of blood and gas is sent into the system, the fluid level increases, lifting the float to the top of the bubble eliminator where a surface such as a 50 A durometer silicon membrane is pressed into a mating component which can vent the pressure in the bubble eliminator. The interaction of the surface contacting the mating component must seal the vent and eliminate the possibility for gas or blood to escape the bubble eliminator. The mating element may be a luer fitting, such as a polycarbonate luer fitting, fixed at the top of the apparatus facing inwards.

The pressure in the system which lifts the float to engage in the seal overcomes the pressure of the patient's blood pressure which is the baseline pressure in the bubble eliminator due to the outlet which connects to the filter and the sheath. This pressure can range from 1 mmHg to 25 mmHg in extreme cases depending on the patient and vein in which the sheath is placed. As the blood level decreases due to the introduction of gas in the system, the float disengages and allows the pressure in the system to equalize and all air to escape through the vent at the top of the bubble eliminator. Due to the patient's blood pressure, the system will backfill with blood from the blood return line and the float will re-engage automatically. As more blood is pushed into the system the blood flows through an outlet at the bottom of the apparatus approximately 0.25 inches above the inlet, but 1.75 inches below the top of the spout on the opposing side of the apparatus. If the float valve fails to disengage and allows for the fluid level to reach below the outlet, a buoyant ball valve will sink with the remaining volume of blood and completely seal the system. This ball is enclosed in a cage directly above the outlet channel and floats to the top of the cage allowing blood to flow freely to the blood return line during normal use. In the case that the blood level does fall to the outlet, the ball will sink with the lowering fluid volume and fully occlude the outlet eliminating the ability for any blood or air to return to the blood return line. When the ball valve is engaged the system will require a user to pressurize the blood return line to disengage it from the outlet channel. The pressure to disengage will vary depending on the pressure imposed on the system when the ball was engaged.

In any of these apparatuses, the ratio of air volume to blood may be significant. The placement of outlet and inlet may be significant. The height of the vertical spout may be significant. The height, volume, density of the float may be significant. The volume of the apparatus may be significant. The dimensions, density, and functionality of the ball valve may be significant. Details of an exemplary deaeration chamber 100 are shown in FIGS. 29, 30, 31, and 32. The deaeration chamber 100 comprises a lower portion housing 102 and an upper portion housing 104 With a blood inlet 106 and a blood outlet 108 formed on the lower housing portion.

A float valve assembly 120 is disposed in the upper portion housing 104 and includes a float element 122, a connecting stem 124, and a resilient seal 126. The float valve element 122 is configured to be buoyant when blood at least partially fills the upper portion housing 104, causing the resilient seal 126 to rise and close against a lower surface of a vent port 128. Closure of the vent port 128 maintains an internal pressure within the deaeration chamber 100 generally equal to the patient's blood pressure when the blood outlet is connected to the patient's vasculature for direct blood return. The resilient seal 126 may be constrained in an upper guide fitting 130 which optionally includes a barb 134 (FIG. 32) for enhancing closure and which aligns the seal with the vent port 128 while allowing the seal to rise and fall to open and close the vent as the blood level in the upper portion housing changes as air separates from the filtered bold over time.

Filtered blood from the system filter (described elsewhere herein) enters the lower portion housing 102 through the blood inlet 106 and is directed upwardly by a flow riser 138 to enter the interior of the upper housing 104, as shown by arrows 150 in FIG. 32. In the upper housing 104, the blood circulates around the float element 132 where it continuously buoys the float element to maintain the seal between the resilient seal 126 and the vent port 128. Any air or other gases entrained (not dissolved) in the blood will separate and collect in the top of the chamber. After a threshold amount of air collects, the float element 122 will drop, allowing the vent port 128 to open and vent the air from the deaeration chamber 100 without disrupting the periodic or continuous blood flow.

### Pressure Sensor at the Syringe

Referring now to FIG. 33, a clot aspiration system 3410 in accordance with the disclosed technology includes an aspiration catheter 3412 having a distal aspiration tip 3414 and a proximal hub 3416. The aspiration catheter 3412 is connected to a pump assembly 3422 comprising a receptacle 3424 having a reciprocating positive displacement element 3226 in an interior thereof. Typically, the pump assembly 3422 comprises a syringe structure including a barrel receptacle and plunger displacement element but could alternatively comprise any one of a number of other positive displacement structures as described with reference to FIGS. 36A to 36F, below.

The pump assembly 3422 is driven by pump driver 3430 which may comprise any of the fluidic, electrical, or other driver mechanisms described elsewhere in this application or generally known in the art. The pump driver 3430 is arranged to reciprocatably drive the plunger or other positive displacement element 3426 via a drive shaft 3432. A controller 3440 is connected to operate and control the pump driver 3430 to reciprocate the positive displacement element 3426 to draw blood into the interior of receptacle 3424 and to expel blood from the receptacle to a blood filter 3442. Flow of blood and clot into the receptacle 3424 and out to the blood filter 3442 is directed by the three-way connector 3420, which typically comprises a pair of one-way valves as indicated by the arrows in FIG. 33.

As described thus far, the clot aspiration system 3410 is exemplary of a number of the systems previously described in the present application. In contrast to the previously described blood pump embodiments, however, the clot aspiration system 3410 further includes at least one pressure sensor 3440 located in an interior of the receptacle 3424 of the pump assembly 3422. By monitoring pressure within the blood and clot receptacle 3424 with the pressure sensor 3440, the pressure induced in the blood within the receptacle by the positive displacement element 3426 (typically a vacuum as the positive displacement element 3426 is retracted to the left as illustrated on FIG. 33 but sometimes a positive pressure if the displacement is abruptly stopped or the direction reversed) can be tracked and used to control the reciprocation rate of the pump assembly 3422.

The pressure sensor 3440 allows the system to measure the vacuum level induced in the blood as the blood is drawn into the receptacle by the plunger or other positive displacement element 3426. Exposure of the blood to excessive vacuum (greater than 100 mmHg) when also exposed to air can cause hemolysis and degradation of the blood when the filtered blood is returned to the patient. Measuring the vacuum allows the controller 3442 to adjust the rate of retraction and/or other position of the positive displacement element 3426 based on the real-time pressure in the receptacle 3424.

While use of the pressure sensor 3440 to directly measure pressure of the blood and clot located in the interior of the receptacle 3424 will generally be preferred, indirect pressure measurement using pressure and force sensors located outside of the receptacle will also be possible. For example, a pressure sensor (not shown) located between the receptacle 3424 and the three-way valve 3420 could also provide a useful reading of the pressure within the receptacle. Alternatively, although less accurately, a force sensor (not shown) on the drive shaft 3432 could be used to indirectly measure the pressure and vacuum within the receptacle.

Note that having the pressure sensor in the syringe allows the system to “stack vacuum” by cycling the syringe, making use of the one-way valves to maintain vacuum at the catheter tip, and maintain vacuum levels higher than is possible with just a single syringe pull.

### Controlling Aspiration Rate and Pressure Patterns

Referring now to FIG. 34, the retraction rate of the positive displacement element 3426 can be controlled based on the illustrated algorithm. When the vacuum protocol is initiated (START), the displacement element 3426 is usually fully to the left, with reference to FIG. 33. The control algorithm will first check to make sure that the displacement element 3426 has not been fully retracted, and assuming it has not (NO), the pressure sensor 3440 will measure pressure within the receptacle 3424. If the pressure is within an acceptable range (CORRECT), the displacement rate will be maintained and the algorithm will again check to see if the retraction has been completed within a specific time increment, typically on the order of seconds. If the measured pressure is high (insufficient vacuum), the rate of displacement of the displacement element 3426 can be increased. If the pressure is low, the rate of displacement can be decreased. The algorithm will continue periodically measuring pressure and adjusting the reciprocation rate as need until the displacement element 3426 has been fully retracted at which time the user may initiate another retraction cycle.

In this way, the aspiration “pull” rate may be increased just to the point of vacuum and no faster, so that the system achieves full aspiration strength without exposing the blood interface to unnecessary vacuum volume. The system can pull just at that flow rate boundary, using the pressure sensor 3440 to provide instantaneous feedback. During pull-back, if the pressure level in the syringe is not yet at the desired vacuum threshold for aspiration, the system could increase the rate of retraction until it reaches the desired threshold, and then maintain that speed while monitoring pressure. If the pressure level indicates more vacuum is achieved than desired, the system could reduce the aspiration pull speed of the syringe plunger, to maintain the desired amount of vacuum in a closed-loop control. Providing such closed-loop feedback control allows the system to maximize flow rate while minimizing exposure to vacuum. The system can keep the blood exposure just at the edge of vacuum and keep the interface fluid-contacting. This can minimize hemolysis of the blood and maintain better blood quality than exposing the blood to large volumes of vacuum and having blood shoot at high speeds into walls of that vacuum chamber.

The clot aspiration system 3410 system may be configured to move the plunger or other positive displacement element 3426 very quickly or abruptly over a series of small increments or steps, as shown in FIG. 35A in full line. Starting and quickly stopping the retraction creates a “positive pressure wave” at the end of each cycle that propagates from the aspiration catheter tip, providing catheter a “water hammer effect” which can loosen clot and promote aspiration into the catheter lumen. This allows a single-direction aspiration system to halt quickly and move and still provide both negative and positive pressure exerted at the catheter tip to help move and loosen any tough clot. This may be done at a frequency rate which is near the mechanical resonance of the system, so the pressure waves may become additive to increase the effective force on the clot. Note that a one-way valve may still be used between the dynamic volume adjustment chamber and the catheter shaft, as long as there is enough hysteresis in the one-way valve to allow the small positive pressure wave through in the reverse direction before closing. See the timing diagrams below.

In contrast to such stepwise aspiration, the displacement and pressure patterns of both a charged aspiration (broken line) and controlled aspiration are also shown in FIGS. 35A and 35B. In charged aspiration, the plunger or other displacement element 3426 is retracted as rapidly as possible (FIG. 35A), exposing the blood to a high vacuum over a very short time period, as seen in FIG. 35B. As discussed previously, such exposure can cause hemolysis. In controlled aspiration, the displacement element 3426 is withdrawn at a rate selected to minimize any vacuum so the blood remains at close to physiological pressure. This is good for the blood but provides little disruption to the clot.

### Aspiration Catheters Incorporating Volume Change Structures

Rather than using a syringe, pump, or other separate vacuum-generating component to draw clot from an aspiration catheter, aspiration catheters of the present technology can be configured to provide a “dynamic volume change” by incorporating a movable wall, barrier, volume change element, or other interface at a proximal end of the catheter configured to create a space or volume to receive the blood and clot flow. Such structures can draw the blood and clot into the aspiration lumen more quickly and with less flow resistance than by using separate syringe or other pump. Such structures can also be used with pressure sensing and control to minimize vacuum exposure of the blood, as described above.

As shown in FIG. 36A, an aspiration catheter 3610 includes a shaft 3712 having a distal tip 3714 and a proximal region 3716 which can be modified in a number of ways to provide for a volume expansion to receive blood and clot through an internal lumen. As shown in FIG. 36A, the volume expansion can be provided by a barrier 3720 which translates in the lumen of the proximal region 3716 of shaft 3712. As shown in FIG. 36B, the volume expansion can be provided by a larger barrier 3730 which translates in an enlarged proximal region 3732 formed contiguously with the shaft lumen. As shown in FIG. 36C, the volume expansion can be provided by a pivoting wall 3740 that opens from the proximal end of the shaft 3712. As shown in FIG. 36D the volume expansion can be provided by a wall 3750 on the end of a bellows 3752 attached to the proximal end of the shaft 3712. As shown in FIG. 36E, the volume expansion can be provided by a balloon 3760 located in a proximal housing 3762 which can be inflated into the catheter lumen and even beyond the distal tip 3714 of the catheter into the vessel which would allow the vacuum-force to be placed as close as possible to the clot itself for improved extraction.

### Smaller Volume Vacuum Chambers for Blood Return

If an aspiration system has a vacuum chamber for aspiration power, if the blood/clot is pulled into that large vacuum chamber it damages the blood due to hemolysis and long-term exposure to the vacuum. If, however, the large vacuum chamber is used to pre-charge a smaller volume chamber with vacuum, and then physically separated from the larger chamber, the smaller vacuum chamber may be used to perform an aspiration of blood and clot without causing much hemolysis. Doing this allows the small volume of blood to be returned to the patient, once the clot has been filtered out of it.

### Vacuum Powered Aspiration Concept

To achieve “BRACE” (Blood Return and Auto-Clot Extraction) with a vacuum source, blood cannot be subjected to sustained vacuum. As shown in FIG. 37, a pump has an inlet pulling air from a chamber effectively creating a negative pressure and an outlet pushing air into a chamber creating positive pressure. The inlet and outlet of this pump are connected to a solenoid or other valve which can switch between the positive and negative pressure lines, imposing different pressure conditions for the chamber. The chamber pictured below is a combination of a syringe barrel (e.g., extraction chamber), a clot canister (e.g., clot filtration chamber), and a bubble chamber (e.g., air separation chamber), or may be any of these. Thus, any of the components described herein may be configured to control the pressure within a chamber of the component to prevent or reduce harm to the blood.

In the figure on the left side of FIG. 37, the system is at a nominal state. When the solenoid is switched, vacuum is created, and blood and clot can be aspirated from the aspiration lumen and pulled into the chamber through a one-way valve on top of a filter or strainer. As the blood fills the chamber it engages with a floating check valve which will automatically seal the chamber when blood fills the chamber which means the blood is not being subjected to further vacuum. This can be seen in the middle figure, where check valve #1 is engaged and check valve #2 is floating. As the solenoid valve is actuated, pressurized air fills the chamber pushing the blood out of the outlet hydraulically. The blood then engages check valve #2 at the outlet ensuring air is not sent from the chamber to the blood return line. As vacuum is applied to the system again check valve #2 will disengage and the cycle can begin again.

This system is a method of creating the same mechanism of efficient clot extraction as the powered syringe with alternative power methods. This chamber may be accessible to access clot or it could be completely sealed and there can be a secondary clot canister-however if that were the case there would have to be a bubble chamber to separate the air that is introduced at the clot canister chamber.

### Deaeration Devices

The key enablement for the dual float mechanism that dynamically filters air from the blood return system is the responsiveness of each float valve (FIG. 38) and the check valve (FIG. 39). The factors that contribute to the responsiveness of the float valve are the exhaust orifice diameter, the mass of the float, and the flowrate of the system. The equations below outline how to calculate the pressure at which the main float will disengage from the exhaust, allowing the bubble chamber to equalize.

$$P = Float Release Pressure$$

$$r = Exhaust Orifice Radius$$

$$M = Float Mass$$

$$G = Acceleration due to Gravity$$

$$A = π {r}^{2}$$

$$F = MG$$

$$P = F / A$$

$$P = ( MG ) / A$$

This action is also what allows air to escape if a large bolus is introduced during standard use. Depending on the flowrate—dictated by the syringe pump return rate, the pressure in the chamber will increase at a specified rate affected by the ratio of air and blood within the system when the return begins. The pressure decay is dependent on resistances past the bubble chamber such as the 40 micron blood return filter, the blood return line, and the introducer sheath where the blood is ported back to the patient. If the system has too fast of a duty cycle where the only fluid entering the bubble chamber is air, the main float will only disengage when the pressure reaches the release point. The check valve may then become engaged if the fluid is completely returned from the bubble chamber and the main float does not disengage. To mitigate the risk of engaging the check valve, intended as a safety, is to optimize the ratio between the exhaust orifice diameter and the mass of the float. The diameter of the exhaust orifice is 0.050 inches and the mass of the float is 17 grams. If the mass of the float-or-the orifice increase the float will be more responsive but the fluid level will become higher to the point where foam or fluid exiting the outlet orifice becomes significantly higher risk. This also means that the chamber will have more fluid inside at a given time.

The responsiveness of the check valve is critical such that it becomes disengaged when fluid enters the chamber. The key parameters that contribute to the responsiveness of the check valve is the buoyancy of the ball, and the ratio of the amount of fluid that can enter the chamber before the main float engages. The more fluid that enters the chamber, the more separation that can be achieved between the ball and the outlet orifice-mitigating the risk of the ball being syphoned into the outlet when the flowrate increases rapidly after a blood return. The buoyancy of the ball may be calculated as a relationship density of the fluid, volume of displaced fluid, and gravitational acceleration using the formula below.

To calculate buoyancy, you can use: B=ρ×V×g, where B is Buoyant force, p is the density of the liquid the object is immersed in (in kg/m³), V is the volume of the displaced liquid, and g is the gravitational acceleration.

In any of the methods and apparatuses described herein removing bubbles (e.g., de-airing the blood) may be part of a closed loop or open loop system. In some cases the removal of bubbles may be automatic. In some cases the removal of bubbles may be manually performed by the user, e.g., using a three-way valve to direct blood to a de-airing chamber as described herein.

Referring now to FIG. 40, the automatic aspiration systems of the disclosed technology may employ a first pressure sensor PS #1 and a second pressure sensor PS #2 to detect system clogging. The first pressure sensor PS #1 may be located in the aspiration catheter handle and the second pressure sensor PS #2 may be located in the syringe. Pressure sensor #1 can detect a clog in the catheter, either at the tip or in the shaft, by sensing a vacuum below a certain threshold at the catheter handle. This sensor can sense when the operator is injecting contrast (or other fluid) into the handle by measuring a positive pressure, thereby helping the sense classification algorithm to not falsely classify increases in impedance due to contrast injection as sensing clot. The pressure sensor #11 may be used in conjunction with impedance sensing to help classify tissue type/catheter tip condition.

Pressure sensor #2 can detect if the syringe is holding a vacuum which can result from for example if: (a) we have pulled back but fluid has not had time to fill the syringe, (b) there is a clog at the catheter tip or shaft, or (c) there is a clog in the tubing between the catheter and the syringe. In addition, the difference in pressure between the two sensors (at the handle vs. at the syringe) can help determine the location of a clog, particularly if it is located between the two sensors.

The information from the pressure sensor #2 at the syringe location can be used to inform the desired position of the syringe plunger. The system can pull the syringe back which performs an aspiration (either upon user button-press or automatically using clot sensing), and then the system choice to initiate the return of the syringe plunger forward can be determined by looking at the data from Pressure Sensor #2. We can keep the syringe held back while vacuum is still present at a certain threshold. Once the vacuum has reduced, we can return the syringe to the forward position. This allows the system to hold onto clot which may be stuck at the tip and also allows the system to continue to ingest clot through the catheter shaft and tubing while vacuum level is sufficient.

Referring now to FIGS. 41A and 41B, the disclosed systems can provide “stacked vacuum” generation where either the system or the user initiates a new forward motion of the plunger and subsequent pull-back to generate a new vacuum. The one-way valves allow for continuous vacuum at the catheter tip during this plunger cycle and allows a way to “top off” the vacuum to the desired level. Fully automatic systems could even maintain a desired vacuum level and automatically perform a syringe plunger cycle to maintain that vacuum if it starts to decrease, as shown in FIGS. 41A and 41B.

In some embodiments, the filter or clot chamber could have a top or lid that can be opened and closed during a procedure to allow clot removal or assessment. The lid may be positioned above the inlet of an aspiration outlet port into the clot chamber. The lid may be able to easily seal and maintain a pressure greater than 5 psi.

An exemplary removable lid assembly 4300 comprising a transparent top 4302 is shown in FIG. 42A. A wiper assembly 4310 for cleaning a bottom surface of the transparent top 4302 is shown in FIG. 42B. The wiper assembly 4310 includes a handle 4312, a wiper blade 4314, and a shaft 4316 that passes through a central opening 4318 in the transparent top 4302 as seen in FIG. 42C. The shaft 4316 is rotatable in the opening 4318 allowing a user to manually turn the handle 4312 to wipe the wiper blade 4314 over the under surface of the top 4310 to clean blood and clot so that the user can see how much clot has collected on the internal filter of the clot chamber.

As shown in FIG. 43, an embodiment of a powered aspiration system 4400 has a disposable unit 4410 and a reusable unit 4420. The reusable unit 4420 houses a pressure source and system electronics. The reuseable unit can be kept outside of the sterile field and is thus suitable for reuse.

FIG. 44 illustrates an embodiment of a powered aspiration system that uses positive pressure house air that is commonly available in hospitals, typically above 80 psi.

As shown in FIG. 45, a powered aspiration system stores negative pressure in at least two chambers where the first chamber has a smaller volume than the second chamber so that the first chamber can be drawn to a vacuum pressure more negative than the vacuum pressure in the second chamber. When the aspiration catheter is near a clot, the aspiration valve can be opened to aspirate blood and at least a portion of the clot into the aspiration lumen of the aspiration catheter. As blood and clot passes through the aspiration catheter lumen and into the first chamber, pressure in first chamber decays, and a pressure valve then closes to the second chamber and opens to atmosphere. In some embodiments, the pressure valve could be closed manually. In other embodiments, the pressure valve could be a float valve that closes automatically. The valve could also be electronically powered and controlled.

Once pressure valve is opened to expose the first chamber to atmosphere, the blood in first chamber can be removed by applying a positive pressure to the pressure port or by applying a pressure source to the blood return valve or blood Return line.

In some embodiments the first chamber includes two separate regions. A clot-receiving region and a filtered blood-receiving region. The regions have a one-valve between them allowing filtered blood to flow from clot region to the blood region.

Thrombectomy Apparatuses with Sensing

As mentioned above, any of the thrombectomy apparatuses described herein, including thrombectomy blood-return systems which are configured to withdraw clot material and blood, remove clot material from the blood and return the blood to the patient, may one or more sensors that may feed into operation of the components (e.g., pump, aspiration catheter, tubing, blood filtering devices, deaeration devices, etc.). Sensors may be configured to detect clot material, to detect blood, to detect air, to detect flow of blood and/or clot material, to detect pressure, etc.

For example, FIG. 9 shows an example of a thrombectomy system 101 including sensing. The primary components in this example may include: the sensing elements (sensors) 152, the aspiration lumen 150, a pressure element 153 (such as a syringe or other motion-type vacuum element), a sensing sub-system 154 (e.g., for interpreting input from the sensors 152), a pressure-element positioner 156 (such as a motion-type vacuum position controller), passive one-way valves to control flow 155, and filtering to separate blood from clot to allow for blood return 159. Filtered blood may be further processed for return from a blood return line 160.

In the simplest case, the sensing interpretation (e.g., the sensing sub-system 154) may be performed by a combination of circuitry/software in the handle and the user utilizing system feedback (such as lights, sounds, or vibrations), and the position control can simply be the user's own hands. This captures the manual-type system useful for pulmonary embolism cases.

The basic thrombectomy system which allows for blood return shown in FIG. 9 may include any of the components described herein. For example, the motion-type vacuum element may correspond to an aspiration devices that include a fluidic driver. The example shown in FIG. 9 includes sensing at the distal end using one or more sensing elements 152 (e.g., electrodes, ultrasound, pressure, etc.). In general, the methods and apparatuses described herein may optionally include sensing at the distal end of the device, or they may be used without sensing. The primary components in this example may include: the aspiration lumen 161 of aspiration catheter 220, a motion-type vacuum or other pressure element 212, a motion-type vacuum position controller 214, first and second valves 226 and 228 (e.g., passive one-way valves to control flow), a filter component 159 to separate blood from clot to allow for blood return 160, and in some examples one or more sensing elements 152 and a sensing sub-system 154 (e.g., for sensing interpretation). In the simplest case, the sensing interpretation may be performed by a combination of circuitry/software in the handle and the user utilizing system feedback (such as lights, sounds, or vibrations), and the position control can simply be the user's own hands. This captures the manual-type system useful for pulmonary embolism cases.

An automated system which removes the user from the sensing interpretation and position control operations is advantageous for procedural efficiency and is further described below. The sensing processing and interpretation may be implemented with a controller 154′, including circuitry and software. The vacuum element may be a syringe and plunger design (either large or small) 163, or a diaphragm type motion vacuum-element. Motion control may be an automatic thrombectomy system which is controlled using the output of the sensing interpretation block. In an automated system using a syringe vacuum element, the primary components may include the sensing elements 152, aspiration lumen 161 of aspiration catheter 220, the syringe with a receptacle and plunger, a control system 154′ with a first subsystem to interpret the sensing information and a second subsystem 156′ to control plunger location, and passive one-way valves 155 to control flow, and filter component 159 to separate blood from clot to allow for blood return 160 as shown in FIG. 10.

In addition to the sensing elements 152 on the aspiration catheter 220, the system may include sensing elements (sensors) within the pump (e.g., the syringe/plunger 163), within the fluid lines connected between the pump and the aspiration catheter or the pump and the filter 159, and/or within the filter 159 and/or blood return line 160. These sensors may be pressure sensors, flow sensors, impedance sensors (e.g., for detecting clot, blood, air, etc.).

Any of these sensing elements 152, along with the sensing circuitry and controller, may determine the aspiration catheter orifice tip condition, such as in blood, touching clot, or touching vessel wall. The sensing elements may be one or more sensing modalities, such as electrical impedance, optical properties, and pressure measurements. This information may be used to control the syringe plunger location to automatically extract clot while minimizing blood loss, and/or to control operation of the pump, filter, etc. The flow rate control of the syringe for clot extraction may be performed between 10 cc/sec and 300 cc/sec for aspiration lumens in the sizes required for human thrombectomy procedures. The flow rate for pushing the plunger forward for blood return may be that same rate or slower (even down to 1 cc/sec), and under system control, in order to reduce hemolysis of the blood and ensure a steady, lower pressure flow back into the body. In the simplest case, the plunger position may be continuously pulled backwards while the tip sensing elements are in contact with clot and stop and/or push forward when the sensing elements are not in contact with clot. This ensures the aspiration lumen 222 is almost entirely filled with clot with very minimal blood, and the blood/clot mixture progresses through the valves 155 (e.g., one-way valves, such as a first valve and second valve) and through the filter component 159 to separate the small amount of blood for return to the patient.

### Additional Sensing

Additional sensing may be placed in the system proximal to the catheter tip, such as in the aspiration catheter lumen, catheter handle, or tubing. This additional sensing may again be one or more of several modalities including pressure sensing, impedance sensing, or optical characteristics. The additional sensing information may be interpreted by the system to help ascertain the condition within the aspiration lumen itself. Particularly, it is beneficial to understand if there is clot within the aspiration lumen of the catheter, whether the aspiration lumen is clogged, or whether the tip is clogged with clot or other material (such as vessel wall).

If the tip sensing indicates the aspiration orifice is in blood, but yet there is negative pressure in the aspiration lumen at the catheter handle, one may deduce the aspiration lumen is clogged, for instance. This information would be useful to present to the user to prompt de-clogging actions such as catheter removal and flushing, or use of a de-clogger tool. If the tip sensing indicates clot-contact, and there is continued low-pressure at the handle, one may deduce the catheter tip is clogged in a “lollipop” scenario, in which case continued vacuum is desired and possibly catheter removal to extract the clot.

Another area for potential additional sensing includes the pressure in the blood return portion of the system. The pressure in that system dictates the rate of blood return to the patient and helps prevent further hemolysis if it is well controlled. A diagram of the system with additional sensing areas is depicted in FIG. 23.

The additional sensing information can add additional layers of complexity and sophistication to the plunger position control algorithm, in both the backward and forward motion. In clogged or “lollipopped” cases the syringe may be held in a far-back position to continue and hold vacuum. In cases where there is already high-pressure build-up in the blood return system, the plunger may be prevented from forward motion and a notification presented to the user. In this case, the pressure may be released by the user or there could be a pressure-release valve. In either case, the forward motion and rate should be a function of the measured pressure in the blood return system.

With the use of pressure sensing information, another way the plunger position may be controlled is to use an intentionally slower aspiration speed to prevent any vacuum gap from occurring during syringe pull-back to help lower hemolysis of the blood. The plunger could be pulled back slower if the pressure appears to indicate good flow through the aspiration lumen, and changed to pull back at a faster rate if the pressure indicates there may be a blockage requiring more force. This adaptive plunger control algorithm based on pressure information may be part of a “Hemolysis Minimization Algorithm”.

Other useful sensors in this system may include a plunger position encoder (already referred to above) to act as an independent confirmation and feedback for the plunger position algorithm, as well as a force-measurement on the plunger motion to check for any error conditions such as a jammed device.

Additional clot-sensing implemented at the handle location of the aspiration lumen can also be useful to help determine optimal plunger positioning. If clot has passed the aspiration orifice but has not yet passed the handle, the system could continue to pull the syringe, for instance.

Rather than just pressure-sensing, flow-sensing may also be implemented which can inform the system of rates into and out of the patient.

### Sensing on the Return Line

In any of the apparatuses described herein, including in particular, systems configured for blood return, it may be particularly useful to include sensing on the blood return line. For example, a powered thrombectomy system, such as the one shown in FIG. 23, may include one or more sensors to determine if there are unwanted materials in the blood return line prior to the blood being re-introduced into the patient. Unwanted materials in this case may include air bubbles and/or blood clot.

Air bubbles in the return line could potentially be of clinical concern for the patient (air embolism), so it would be best to connect the detection of bubbles to some type of system notification (lights and/or sounds) similar to a bubble alarm in other medical devices (such as an IV line).

Clot in the return line could also cause clinical issues if the clot were to enter the patient and not be otherwise filtered before patient entry. Even if clot did not enter the patient, it would still be useful to know if there was clot in the return line to allow the user to flush it out as required to ensure consistent blood return flow rates, as clot could block and/or clog filter components. In any of these cases, the apparatus and method may include user notification (lights/sounds).

To assist a powered thrombectomy system with additional information about bubbles and/or clot in the return line, additional sensing may be placed along the return line. The return line is typically a positive-pressure side of the system, and may include tubing to couple to the patient, at the same or a different location than the aspiration catheter. Sensing could be selected from any of several different modalities, but could include impedance sensing using electrodes along the blood return path, optical techniques, ultrasound sensing, etc. Electrodes could be fixed shapes or rings, and optical methods could include light either passing through the returned blood line or reflected from it. Either of these methods would be able to detect the difference between blood and either clot or air bubbles. Sensing could be placed either before or after filtering components in the return line. In some cases sensing (e.g. one or more sensors) may be placed before any final filter components, so if there is any clot it is sensed and captured. In the example show in FIG. 23, a sensor 754, 755 may be included on the return line before 754 and/or after 755 a filter and/or de-airing device (not shown).

### Real-Time Sensing

In general, the methods and apparatuses described herein may include real time or near-real time sensing. In particular the methods and apparatuses described herein may be configured to sample tissue, process the sampled data and determine clinically relevant information regarding the tissue within real or near-real time. This sensing may provide essentially real-time feedback of the classification of material at the catheter tip and/or within the system which may be critically helpful during a procedure. Thus, the methods and apparatuses described herein may include sensing in which the response is under about 1 second or less, (e.g., sensing a change in condition), and preferably within 500 ms or less (e.g. 400 ms or less, 300 ms or less, 200 ms or less, 100 ms, or less, etc.). This allows for the catheter to be moved by the user (e.g., physician) and the user may be informed of any changes, such as touching clot, or latching onto vessel wall, within a timeframe useful for human responsiveness to guide the procedure.

Thus, the hardware and software of the apparatuses described herein may be configured to meet these timing goals. In general, higher frequencies (e.g., 50 Hz or greater, 100 Hz or greater, 150 Hz or greater, 200 Hz or greater, 300 Hz or greater, 400 Hz or greater, 500 Hz or greater, 1 kHz or greater, etc.) may be used so information is ascertained faster, as well as higher data rates being sampled. If clot material is moving (e.g., through the catheter tip or shaft), a higher sampling data rate may be used; for example, the apparatus may be configured to sense/sample every 10 ms (100 Hz) or faster.

### Blood Return Options

The apparatuses (e.g., device, systems) described herein may implement various ways of returning the blood, such as a manual return, e.g., using a syringe which is available for the user to take the filtered blood and return it to the patient through an available port. A preferable system may automate that process and have the system automatically return the blood to the patient through a direct line. The same syringe-based system with pressure monitoring may be used where the pressure/flow back to the patient is monitored and controlled. This again helps reduce hemolysis and ensures there is a metered and safe flow rate for the patient. The total amount of time the blood has been outside the body may also be monitored, and if it exceeds certain limits (such as 5 minutes) there may be actions taken to warn the user or prevent the return of that blood as it has increased risk of clotting. In either case, the system with automatic blood return may include bubble-elimination elements to ensure no air is fed back to the patient. FIG. 46 illustrates an example of such a system.

### Fluid Circuit

As described and illustrated above, the methods and apparatuses shown may be configured to form a closed-loop blood return circuit, which may include a source positive and/or negative pressure to drive both aspiration and return of blood to/from the patient. Blood and clot material may be aspirated from the body, filtered, de-aired, and returned back to the body. The closed-loop blood return circuit may be referred to as a fluid circuit or blood loop, and forms the clot management system.

In general, the apparatuses (e.g., devices, systems, etc.) described herein may include the use of a compressible fluid (e.g., air) within the blood return circuit to ensure clot visibility and moving of blood back to patient (sourced positive air pressure). In some cases the compressible fluid may be present within the clot removal container.

Any of these apparatuses and methods may include one or more features to reduce or prevent clotting within the blood return circuit, and in particular the portion of the return line portion of the blood return circuit. For example, these methods and apparatuses may include flushing of the blood return line. The portion of the blood return circuit being flushed may extend over all, or a portion, of the circuit between the source of driving (positive) pressure, such as the powered syringe or other positive pressure source, and the point of patient blood return (e.g., catheter, sheath, etc.) into the body. For example, the portion of the blood return circuit being flushed may be the portion between the clot removal chamber (e.g., clot cannister) to the point of patient blood return, or in some cases the portion between the de-airing chamber (e.g., bubble chamber) and the point of patient blood return, etc. Flushing may be automatic or manual, or semi-automatic. In some cases flushing may be continuous or for a predetermined period of time (e.g., 1 second or less, 2 seconds or less, 3 seconds or less, 5 seconds or less, 10 seconds or less, 15 seconds or less, 20 seconds or less, 30 seconds or less, 1 minute or less, 2 minutes or less, between 1 second and 5 minutes, between 1 second and 2 minutes, between 1 second and 1 minute, etc.). Automatic flushing may be triggered based on elapsed time, time since the last activation of the source of positive pressure, etc. In some cases automatic flushing may be triggered based on sensed blood within the blood return circuit (and in particular within portion of the fluid circuit to be flushed, such as the blood return line).

Any of these methods and apparatuses may include one or more additional pressure source and/or source of heparin. In some cases these methods and apparatuses may include a port into the fluid circuit for applying fluid (e.g., saline) that may be used as a wash or flushing fluid. For example, these methods and apparatuses may be configured to deliver additional fluid, such as saline (or saline with heparin) into a port (e.g., a flush port) on a return side of blood return circuit. Alternatively or additionally the blood return circuit may include a coating with one or more anti-coagulation agents, such as heparin. In some cases the blood return line may include an anti-coagulation coating.

The blood return circuit may include one or more sensors. For example, the blood return circuit may include one or more bubble sensors and/or clot sensors to detect air bubbles and/or clotting within the blood return circuit. In some cases the apparatus may include one or more impedance or optical sensors monitoring the blood return circuit (e.g., a fluid line or path of the blood return circuit).

Alternatively or additionally, these method and apparatuses may detect or sense clot, vessel wall and/or blood at the site of aspiration, such as the distal end region of the aspiration catheter. Any appropriate sensor(s) may be used. Examples of aspiration catheters including one or more sensors that may be used with any of the apparatuses and methods described herein may include but are not limited to those shown and described in U.S. patent application Ser. No. 17/861,082, titled “APPARATUSES AND METHODS FOR DISTINGUISHING CLOT MATERIAL FROM VESSEL WALL, filed on Jul. 8, 2022, U.S. Pat. Nos. 11,730,924, 11,730,925, and PCTUS2023086322, titled “CLOT SENSING METHODS AND APPARATUSES, filed on Dec. 28, 2023. Each of these patents and patent applications is herein incorporated by reference in its entirety.

Any of these methods and apparatuses may include one or more sensors to detect when the aspiration catheter latches onto a wall of the blood vessel (vessel latch) and may be configured to release vacuum automatically to unlatch (e.g., automatic release). For example, latching may be detected based on the impedance and/or optical properties. For example, vessel latch may be detected by detecting an impedance that is distinct form the impedance of blood and/or vessel. In general, these apparatuses, including in particular the aspiration catheter, may include one or more markers (such as radio-opaque markers) at or near the distal tip, and in particular at or near the aspiration opening; these markers may indicate tip orientation (e.g. orientation of the aspiration opening).

Any of the methods and apparatuses described herein may be configured to communicated wirelessly or via a wired connection to a display and/or remote processor and/or a memory external from the blood return circuit. For example, any of these apparatuses may be configured to broadcast data to a display and/or external (wireless, Bluetooth, etc.).

### Compressible Fluid

As discussed above, the blood return circuits may be configured to use a compressible fluid, such as air (e.g., air, CO₂, O₂, N₂, etc.) as part of the blood return circuit to allow cushioning of the blood when driving it through blood return circuit. In some cases the blood return circuit includes one or more (e.g., two or more, three or more, etc.) positive air reservoir to allow the user to access and visualize the clot, while also assisting in the continuous return of blood by using the compressibility of the air as a capacitive spring which can store energy when pressurized, then applying a pressure within the system returning the blood at a flowrate not dependent on the rate of blood entering the positive pressure side of the system.

FIG. 47 illustrates one example of a blood return system 4700 (e.g., a blood return circuit) that includes a pneumatically driven syringe plunger 4707 that is actuated to create negative pressure within a syringe during a first stroke that communicates with an aspiration lumen 4709 that is continuous with an aspiration catheter 4705 through an un-interrupted fluid column extending from the face of the plunger to the distal tip of the aspiration catheter, through a first one-way valve 4711. When actuated, this syringe may create negative pressure by rapidly increasing the volume of the chamber, entraining blood and thrombus into the aspiration lumen and syringe body. When the plunger is returned, the blood and thrombus are routed to the blood return portion of the clot management system through a second one-way valve 4713. The second one-way valve separates the dynamic syringe, which can create both negative and positive pressures, from the blood return portion of the clot management system (e.g., the blood return circuit) which can only be subjected to positive pressure by the syringe return. The first one-way valve 4711 prevents positive pressure from the syringe driving blood back out of the aspiration catheter. Ball check valve 4737 controls blood return through blood return tube 4735.

The blood return portion of the blood return circuit may include a clot canister 4717 containing a filter/strainer to separate thrombus from blood. The clot canister has an inlet that is above the filtration elements and an outlet at the lowest point of the chamber, below the filtration element. The clot cannister 4717 is connected to the source of positive pressure via a positive pressure line 4715 that includes a positive pressure lumen.

The clot canister may include an air reservoir 4739 (“volume 1”) within the clot cannister 4717. The outlet of the clot canister connects to the inlet of the de-airing chamber (e.g., bubble removal chamber 4721) and may be separated by a third one-way valve 4723 that allows for flow from the clot canister to the bubble chamber but not reverse. As described above (e.g., in reference to FIGS. 26-32), the bubble removal chamber, which may also be referred to as a de-airing chamber, is configured to separate air from the filtered blood and ensure that only filtered blood is returned to the patient and no air can enter the blood return line leading to a tertiary filter 4725 and then the access point to the patient. The bubble chamber may include a dynamic float (e.g. float valve 4727) that seals against an exhaust vent 4729 at the top of the chamber and when at atmospheric pressure rests in the system, allowing the exhaust vent to remain open.

The float (e.g., float valve) 4727 may include an elongated neck that protrudes above the fluid level which ensures a cavity of air is trapped within this system above the inlet. This secondary air cavity 4731 may work in conjunction with the initial air cavity 4719 within the clot canister 4717 to create a capacitive positive pressure store to push blood through the system after it is returned by the syringe. The stored air 4719, 4731 may allow visualization of thrombus material, may allow access to thrombus material when the clot canister is opened, and may allow for the system to pressure rapidly so it can accept large volumes of fluid quickly, while filtering and returning the blood to the patient slowly.

In this example, the air pocket (e.g., the air pocket in the clot collection chamber, volume one 4719) compresses as blood enters the chamber, which in turn assists the flow of blood through the system as it applies a positive force to the blood within the chamber, driving it through the clot collection chamber 4717 (and filters) and into the bubble chamber 4721 where any air that may have entered the system can be separated from the blood that will be returned to the patient. The bubble chamber is separated from the clot canister by a one-way valve 4723 that allows for both chambers to increase in pressure equally as blood enters the system but allows for the bubble chamber to remain at sustained positive pressures and continue to return blood to the patient when the clot canister is opened. The clot canister pressure may equalize to atmospheric pressure. This occurs when the user wants to inspect and/or remove thrombus material from the clot chamber 4717.

FIG. 48 shows subsystem 4800 (e.g., an empty blood return portion of the fluid circuit of FIG. 47) with the inlet to the clot filtration chamber being the highest point fluid can reach within the system. The line 4844 indicates the top surface where fluid will fill in the volume 4852 when the system is at low pressure (e.g., pressure that is greater than atmospheric pressure). The height 4846 represents the displacement of the inlet from the clot canister 4717 to the outlet of the bubble chamber 4721, which is the lowest point of the blood return system. This is relevant for characterizing the fluid column pressures that allow blood to flow from the first chamber 4717 to the second 1721 chamber. The height between the top surface 4844 and the fluid line between the clot collection chamber 4717 and the bubble removal chamber 4721 (e.g., height two, 4848) and the height 4850 between the top of the opening into the bubble removal chamber and the de-aired blood exit at the bottom of the bubble removal chamber (height three, 4850) are variable depending on the pressure within the system. The blood that enters the blood return portion of the fluid circuit flows from the clot collection chamber 4717 to the bubble removal chamber 4721. Once the fluid reaches the outlet of the bubble removal chamber 4721, the bubble removal chamber 4721 begins to fill due to the inlet flow being greater than the outlet flow out of the bubble removal chamber 4721. The air being displaced travels out of the exhaust vent 4729 at the top of the chamber until the elongated neck of the float contacts the vent creating a seal. The volume of air trapped above the fluid becomes a capacitive air reservoir labeled volume three 4719. When the float seals against the exhaust vent, the pressure in that chamber will increase as new fluid enters at a rate greater than what is exciting the outlet of the chamber. The clot collection chamber 4717 (e.g., clot canister) will begin to fill as well due to the resistance at the outlet one-way valve. Up to this point the clot canister has not been filling, but instead allowing the filtered fluid to flow directly to the bubble chamber 4721. As the fluid begins filling the clot canister 4717, all air in that chamber becomes trapped above the fluid level. This becomes the capacitive air reservoir (volume one) 4839. FIG. 48 also illustrates clot filter 4841.

The float 4727 within the bubble chamber 4721 acts as a valve on the vent allowing this chamber to exhaust surplus amounts of air and equalize the system in the event that air is introduced. Air can be introduced to the system when a user opens the clot collection chamber 4717 to access clot, or if the device is used improperly and air enters the aspiration lumen. The float 4727, when resting as shown in FIG. 48, does not trap an air reservoir. FIG. 49 shows subsystem 4800. Only when fluid enters the system as shown in FIG. 49 does there become a trapped air cavity. The elongated neck 4726 of the float 4727 protruding from the fluid creates a separation between the fluid, and the exhaust vent which holds air until the system depressurizes due to fluid exiting the outlet and the float releases. The air cavity within the clot canister serves at least the three primary functions described above (e.g., allowing visualization, opening of the clot collection chamber, and accept large volumes of fluid quickly while releasing slowly), while the volume of air 4719 in the bubble chamber 4721 serves allows compressibility, similar to the volume of air 4739 in the clot collection chamber 4717, however the primary function of trapped air in the bubble removal chamber 4721 is to allow for the chamber to become pressurized rapidly and to apply a positive force to the blood, moving it through the blood return line and the tertiary filter before it is returned to the patient.

If these volume of trapped air did not exist in the system, as the aspiration syringe returns the fluid to the blood return system, a uniform pressure from the syringe plunger head all the way to the tertiary filter would limit the speed of return to the minimum flowrate through the system (e.g., approximately 15 ccs). This would limit the user's ability to aspirate and return blood at a rate that is desirable for the effective extraction of thrombus from the patient. Without the volume of trapped air, the increased pressurization and lack of compressibility in the system would impact blood quality as flowrates increase and pressures remain a constant high until the exact volume that enters the system is returned to the patient. The relative incompressibility of blood means that adding a capacitive air cavity in these particular locations allows for a closed loop blood return system to have multiple segments that can allow for visualization and access to blood and thrombus during a case without need for flushing or prepping the system. FIG. 49 shows an example of a system full of fluid with an air reservoir trapped in each chamber.

A capacitive air reservoir as part of the blood return portion of the fluid circuit may be used in any of the methods and apparatuses described herein, including in implementations where there is a single chamber or a plurality of air chambers. In an embodiment with a single chamber, the trapped air cavity can function the same way allowing for a volume of air to become compressed to allow for large volumes of fluid to enter a chamber quickly, even if the flowrate entering the chamber is significantly greater than the flowrate out. In examples in which a powered syringe system is used, the system may have an inlet flowrate between 6-40 ccs/s with peak instantaneous inflows up to 200 cc/sec and an outlet flowrate between 6-12 ccs/s. The aspiration sequence may drive the blood return pressure and flowrate as the aspiration of blood from the aspiration lumen into the syringe may move all blood to the blood return system before performing a second aspiration indicated by the head of the syringe plunger reaching the front of the syringe barrel.

### Smart Fluidic Driver System

The methods and apparatuses described herein may remove thrombus from patients' vasculature though a catheter and aspiration source for treatment of disease states such as deep vein thrombosis (DVT) or pulmonary embolectomy. As described herein, these methods and apparatuses may facilitate the removal of blood and thrombus, the separation of blood from thrombus (clot material), and then prepare and/or deliver the blood (manually at the hands of the user, automatically or semi-automatically) to the patient. As described above, these steps may be integrated into one apparatus (e.g., one system) for improved procedural efficiency and better patient safety; any of these apparatuses may be configured to provide information to the users to improve their decision-making process and ultimately patient safety.

For example, any of these methods and apparatuses may include the use of a smart fluidic driver not only aspirate and remove the blood and thrombus from the patient, but also to actively control the filtration and subsequent return of the patients' blood back to the patient, providing a “smart” fluidic drive system. Thus, any of these apparatuses may include one or more of an aspiration piston (e.g., aspiration syringe), a pneumatic actuator driving the aspiration piston, pneumatic regulators, pneumatic variable flow restrictors, pneumatic valves, a pressure source (e.g., a source of positive pressure), one or more pneumatic pressure transducers, and electronics configured to coordinate operation of these system, including these components.

For example, any of these apparatuses may include an aspiration piston (e.g., an aspiration syringe) that is configured to apply aspiration through an aspiration catheter to aspirate blood and clot material as described above. In some examples the aspiration syringe includes a syringe-like system comprised of two independent inlets and outlets with a pressure transducer port within the fluid contacting portion, a barrel, and a piston. The inlet and outlet of the syringe may also include opposing one-way valves to regulate the direction of flow into and out of the syringe.

As mentioned, any of these apparatuses and methods may include the use of electronics (e.g., one or more controllers, processors, memory, etc.) which may include embedded software control. For example, the electronics may include electro-mechanical components which can record, monitor, adjust, and/or activate actuator position and motion, pressure transducer data, valve activity, regulator adjustment, and/or time.

Any of these methods and apparatuses may include a pneumatic actuator, such an actuator having an inlet and outlet port and a sliding piston between the ports connected to a shaft which extends and retracts as each side of the actuator is pressurized and/or exhausted. This shaft is coupled to the aspiration syringe plunger.

These methods and apparatuses may include one or more pneumatic regulators, which may include mechanical and/or electro-mechanical driving fluid pressure regulators. These apparatuses may include multiple of these components to have different pressures at different sides of the pneumatic actuator. These apparatuses may also include one or more pneumatic variable flow restrictors, including mechanical and/or electro-mechanical flow restrictor which allows for an adjustable orifice size for the pressurized fluid to pass through on its way into or out of one or both sides of the valves and/or the actuator.

Any of these apparatuses may include one or more pneumatic valves. Pneumatic valves may include electronically and/or pneumatically controlled valves to control directional flow of driving fluid from the pressurized source to the pneumatic actuator based on electronic from the electronic components.

Any appropriate pressure source may be used to drive the pneumatic components, and in particular the pneumatic actuator. For example, the apparatus may include a compressible fluid stored in the system and accessible by the pneumatic components to drive motion through the potential work of the pressure differential relative to atmosphere, such as a source of compressed air, CO₂, etc.

In addition, any of these apparatuses may include one or more pneumatic pressure transducers. Mechanical and/or electro-mechanical pressure transducers may be in-line with the pressurized driving fluid, which can inform the system of pressure at its location. If placed at the pressurized fluid source, this can inform the system of the amount of source pressure is left for use. If these sensors are placed at the regulated side of the pressure regulators, then the outputs of these sensors can be used to adjust the regulators to desired thresholds.

These components (e.g., aspiration piston, a pneumatic actuator driving the aspiration piston, pneumatic regulators, pneumatic variable flow restrictors, pneumatic valves, a pressure source, one or more pneumatic pressure transducers, and electronics) may be part of a “smart” fluidic driver that is interconnected mechanically and electronically, and may provide an system that can adjust itself actively according to the various states of the inputs and can allow for more unique output control states and more knowledge of the state of the system at a given moment, as described herein.

In some cases these systems including smart fluidic drivers may provide a variable aspiration rate. These apparatuses may control the orifice size of the pressurized driving fluid going into the growing side of the actuator and/or the shrinking side of the actuator upon initiation of actuator movement, thereby precisely controlling the rate of the actuator. In some examples the apparatus may have one or more predefined user-controllable aspiration rates. In some examples the system may use the pressure data from within the syringe barrel to automatically and actively adjust the orifice diameter during the aspiration stroke, to ensure the optimal aspiration rate is achieved for a given aspiration. The ability to adjust the aspiration rate actively is valuable for multiple reasons, such as, but not limited to the limitation of the quantity of blood being exposed to vacuum for blood quality purposes by only aspirating as fast as necessary to fill the syringe, but not any faster so as to not create a vacuum chamber within the syringe. In the case that there is thrombus starting to collect within the catheter or its tip and therefore no more fluid filling the syringe, the active orifice could close and/or the valve and prevent further aspiration. This would help with efficiency of the system and the procedure but also limit the blood exposure to vacuum and thus reduce the degassing and blood degradation. As the clot gets entrained, the valve and/or orifice control could allow for the aspiration to continue until the desired (by the user) aspiration volume is reached. Additionally, in the case that different size (internal diameter or length) are used, there are limitations on the flowrate that can be achieved through those catheters given a particular media (in this case blood, but could also be saline), and so by controlling the aspiration rate at the fluidic driver, the system could adapt the rate for each given catheter size to make a more efficient system and improve the quantity of aspirations available for a given pressurized chamber volume.

The systems including smart fluidic drivers described herein may provide a variable return rate. Similar to the aspiration rate control described above, the same concepts and components, when positions on the opposing side of the actuator, can restrict the rate at which blood is returned to the patient. Thus, the user may adjust the rate of blood return to ensure patient safety, which can be tuned for patients with different risk indexes or other health factors which may drive the rate at which blood can be returned to their vasculature. Additionally, with knowledge of the pressure within the syringe and within the patients' vasculature (e.g., via the pressure transducer within the catheter), the system could adjust the rate of return of blood as a function of the rise or not of the patient's vascular pressure, so when the return is not causing a significant rise in vascular pressure. For example, the orifice can be opened for the return rate to be increased. Should the pressure in the patients' vasculature rise significantly upon return, the system can restrict the orifice driving the rate of the return of the syringe.

### Flow Rate

The apparatuses (e.g., systems, including powered systems, close-loop systems, etc.) described herein may be configured to achieve relatively high flow rates without using a flow control device. This may be due, in part, because the apparatus may be configured to have a flow circuit that does not restrict the fluid path between the aspiration catheter and the pressure source. Further, the lumen along this path (e.g., the entire lumen) may be configured to have the same size (e.g., diameter) as aspiration lumen or may be larger. For example, the minimum diameter along the flow path between the aspiration catheter rand the pressure source maybe set by the diameter of the aspiration catheter. The flow path between the aspiration catheter and the pressure source may be configured so that it does not bend or curve with a radius of curvature of less than a minimum amount, e.g., 1 mm or less, 7.5 mm or less, 1 cm or less, 1.5 cm or less, 2 cm or less, 3 cm or less, 5 cm or less, etc. (and preferably 1 cm or less, 2 cm or less, 2 cm or less, etc.). Thus, in some cases these apparatuses may be configured to have flow properties as described in Table 1, below:

TABLE 1
Flow rates

| | Max | Avg. | Time to Fill | Energy |
|---|---|---|---|---|
| | Flowrate | Flowrate | Syringe | required |
| | [cc/sec] | [cc/sec] | [secs] | [Joules] |
| 20F Aspiration | 181 | 120 | 0.494 | 1.94 |
| Lumen | | | | |

The example values given in Table 1 are for illustration only. For example, the max flow rate may be between 150-200 cc/sec (e.g., between 160-190, between 165-190, between 170-200, between 170-190, between 180-200, etc.), the average flow rate may be between 100 and 160 (e.g., between 110-150, between 110-140, between 110-130, between 100-150, between 100-140, etc.) cc/sec. The time to fill the syringe may be between 0.4-0.6 sec or faster (e.g., between 0.3-0.6 sec, 0.25-0.65 sec, 0.2 to 0.6 sec, etc.).

Additional features may be added to the system described above, e.g., to handle the case where the plunger reaches the end of the syringe.

Obviously, some additional complexity must be added to the system described above, at the very least to handle the case where the plunger reaches the end of the syringe. The simplest control method in this case is to simply return the syringe to the forwardmost position and continue. This is shown in the plunger position timing diagram in FIG. 19.

Valves, such as one-way valves including a first valve and second valve that are configured to allow the pumps described herein to both withdraw blood and clot material and to pump filtered blood back into the patient (or into a container for later re-introducing into the patient) may be simple passive duck-bill style fluid valves or have more complex structures. Key aspects of the design may include the ability to operate and fully close even when there is a mixture of blood and clot. Valves may either be very strict or may include a small amount of hysteresis of reverse flow prior to closing.

Any of these systems including smart fluidic drivers may also be configured to provide a variable aspiration force. For example, using the mechanical and/or electro-mechanical adjustable pneumatic regulators, the system can be tuned by the user, or actively tune itself to adjust the force that the actuator can move in the direction of aspiration. This may be advantageous in the case of sensitive vasculature at the catheter tip where vessel trauma is of concern. In any of these examples the system can actively reduce the pressure of the driving media to the actuator and thus reduce the aspiration force. On the contrary, if the user wants to select a more powerful aspiration and/or if the system detects insufficient vacuum within the syringe and/or if the system begins to present higher frictional forces as seen by a reduced rate at the position encoder on the syringe/actuator, the pressure of the driving fluid can be increased to allow for up to about-1 atm of vacuum force from the syringe onto the blood.

Any of these systems having smart fluidic drivers may be configured to provide a variable return force. Similarly to the variable aspiration force, the systems described herein can control the force of return out of the syringe and into the filter and patient through a mechanical and/or electromechanical adjustment done by the user or actively by the system. Unlike the aspiration force, the return force may be a positive pressure within the syringe which can be increased beyond the 1 atmosphere that the vacuum side can theoretically attain. This option for increasing return pressures allows for a faster flowing and more powerful return stroke of the syringe, thus pushing the clot into the filter and through the resistances of the blood return loop, back to the patient. If the patient pressure does not rise and the orifice restrictor from the return rate control is maximized, then the driving fluid pressure can be increased to allow for increasing return rates. Additionally, as the system gets used and more clot is collected within the system and the filter, the return of the blood and clot from the syringe to the clot filter and of the blood from the clot filter to the patient will increase, and thus more pressure may be required to ensure sufficient return rate. Alternatively, if the system is fast flowing, then the return pressure can be actively reduced to conserve the volume of pressurized fluid and allow for more use of the system for a given pressurized fluid source volume. The systems described herein may be configured to perform these adjustments automatically.

Any of these apparatuses (e.g., systems) may be configured to detect an insufficient source pressure. There are multiple ways to detect insufficient source pressure that may be performed by these systems, including actively monitoring the pressure using a pressure transducer at the source vessel. However, in some cases the additional components required can add cost and complexity. Using the actuator/syringe position encoder alone and/or combined with the syringe pressure data, the system can detect a reduction in source pressure through a decrease in return rate of the actuator (assuming no other valves, regulators, or orifices are changed), or an increase in the time to complete a return stroke. However, the rate or time alone may not indicate if the system is running out of source pressure, because the reduction in rate can be caused by the return loop resistance increasing, the inability for the patient to intake the returning blood, and/or the clot filter getting obstructed. In those cases, as the return stroke may be initiated, the pressure in the syringe increases as the stroke is returning, however in the event of a decrease in source pressure, the return stroke rate would decrease (or time for the return stroke would increase) and the syringe pressure would not increase at all or not as high. Therefore, the combination of a slowed return stroke with a lack of relative rise in syringe pressure may be used by the system to detect a loss of source pressure and could be used to indicate to the user that a new source pressure vessel is needed. This system configuration may allow for increased efficiency of the procedure by earlier detection of a decrease in source pressure but also be more cost effective and less complex mechanically than placing a pressure transducer at the pressure source vessel.

Any of these apparatuses may also or alternatively be configured to detect a clog during aspiration. For example, the system, as described, may have the necessary components to be able to automatically identify clogs within the aspiration system. If the pressure in the syringe is relatively low (vacuum), and the syringe is not actively moving (based on encoder data), then there is no blood entering the inlet of the syringe and therefore there is an obstruction ahead of the syringe. With use of the pressure transducer in the catheter, when there is no differential with the catheter pressure and syringe pressure, then the clog is distal of the pressure transducer in the catheter. When there is a pressure difference between the catheter pressure and the syringe pressure (the catheter is at a higher absolute pressure than the syringe), then there is a clog between the syringe and the catheter. Both of those situations can be used to inform the user of their next actions and improve patient safety such as, but not limited to, not needing to remove the catheter if the clog is not in the catheter. These situations have been tested and visualized, with objective identification of the location of the clog, and through a variety of possible user interfaces, could help further define the clinical situation to the user and/or help troubleshoot when in a clogged state.

Any of these apparatuses (e.g., systems) may be configured to detect occlusions at the level of the filter. For example, it may be helpful to let a user know how much clot is being captured and when the filter may need to be replaced. From a procedural efficiency, it could be beneficial to the user to not need to verify the volume of clot being aspirated after every single aspiration, and therefore an indicator that would allow the user to not have to change their procedural steps could help improve procedural efficiency and offer reduced procedural time. Alternatively or additionally, this can be used to better understand the clot burden that is being removed from particular locations of the patients' vasculature and help inform the level of response and action the user may want to consider continuing or not. This can be detected through an increase in pressure in the syringe during a return relative to a starting pressure prior to the aspiration. As the differential in pressure between the starting baseline pressure and the return pressure increases, there is more and more obstruction or resistance in the filter which would result in higher return pressures for a given return time. Additionally, because monitoring of the pressure is also in the catheter, which is directly connected to the patient vasculature, this catheter pressure can be used to infer vascular pressure. As a return is being conducted, there may be a localized and temporal change in pressures due to the returning fluid, as there may be a reduction in vascular pressure upon aspiration. This rise in vascular pressure can be used by the system in conjunction with the syringe pressure to understand the level of occlusion in the blood return loop and/or the clot filter(s). As the filter is more and more occluded, the vascular pressure would not have the same pressure signal at given syringe pressures. If the syringe pressure is very high, the system may predict a certain level of rise in the vascular pressure, but if that same level of rise in vascular pressure is not detected, the system may then conclude that the filter is clogged, and the high syringe pressure is only up to the filter and that the fluid from the filtered side of the filter to the patient is low. This information can be used to help inform the user of when the filter(s) should be cleaned out or replaced.

Any of these methods and apparatuses may be configured to identify the volume of clot aspirated. For example, the apparatus may be configured to place a small length of reduced diameter in the blood path between the syringe outlet and the clot filter and using the syringe pressure and syringe/actuator encoder position information, the amount of restriction through that orifice can be quantified from both a return stroke and syringe pressure perspective for various clinical situations. When the return stroke of the syringe contains only blood, the syringe return rate, and the pressure curve will have a specific profile. In the event that there is clot mixed in with the blood, as the clot passes through the known restriction in the path between the syringe and the clot filter, the stroke rate may be reduced and/or the pressure increase in a non-uniform way, which depending on the chronicity and size of the clot, will have a unique pressure profile and syringe return stroke profile. Through testing each unique clot size and clot types can be characterized through the restrictor, thus allowing the system to be able to quantity and qualify the clot that is aspirated into the system. This information can be vital to the user to better understand the patients' disease(s), symptoms, and level of care that may be required. Additionally, this data can be collected on a large number of patients to better understand population-wide trends or correlations to clot types and locations to better information the doctors and healthcare industry.

Any of these methods and apparatuses may be configured to identify the volume of blood and/or clot aspirated. For example, the syringe pressure and the syringe/actuator encoder position data in combination may be used to detect how much blood and clot is being aspirated and returned through the system due to a detectable and unique signal profile. Following an aspiration without the user intervening, the syringe may be filled with blood anywhere from 0-100% of its total possible volume. Upon return, if there is a combination of vacuum and blood/clot in the syringe, the return rate of the system will have a unique profile due to the vacuum force aiding in the return stroke in combination with the pneumatics. This may be detected by a change in stroke rate, while the syringe pressure is measuring vacuum. Once the syringe is no longer measuring vacuum at some point during the return, then at that time there may be a change in rate of the return stroke (as observed by the encoder data), which would then complete the return until the front position. In the event that the entire syringe is only vacuum and there is no blood/clot in the barrel of the syringe (due to a clog in the catheter or somewhere distal to the syringe), then the entire return stroke will be aided by vacuum and the encoder return rate will be relatively fast. However, in the event that half of the syringe is vacuum, and half is blood/clot, then upon return, the vacuum will assist the return stroke for half of its stroke length, and at that position, the return rate will change until the stroke is completed. The location of this change in rate and the change in syringe pressure from negative to positive (relative to atmosphere) can be used to inform the system and/or the user of the amount of blood/clot that is being returned and therefor has been aspirated from the patient. In the event that the prior feature (Clot aspiration volume identification) is also implemented, then in combination with this feature, the differentiation between blood and clot can be identified and therefor the exact volume of blood and volume/type of clot that is being removed from the patient can be collected by the system. This information can be useful to the user to track the hemodynamics of the patient and improve patient safety, in addition to the data from case to case to be compared and larger population or user specific trends identified to improve the standard of care. This example is illustrated in the graph shown in FIG. 50. In this example, the left y-axis represents the pressure in mmHg, the x-axis represents the time in milliseconds, and the right y-axis is the encoder voltage with the higher voltages representing the syringe in the front position, and the lower voltages representing the syringe in the retracted position. As seen in FIG. 50, there is a change in rate when the encoder voltage is at 2.15V, which represents 86% of full scale or in this case 5 cc. In this example the syringe had 14% blood and 86% vacuum at the time of start of return. This was confirmed by the user as tested on the benchtop. This is one example; however further testing can confirm the location of change for various amounts of known volumes in the syringe to educate the system of what percentages of full range of encoder voltages equate to how much volume.

The methods and apparatuses described herein may identify captured clot volume. For example, by placing a small length of reduced diameter in the blood path between the syringe outlet and the clot filter and using the syringe pressure and syringe/actuator encoder position information, the amount of restriction through that orifice can be quantified from both a return stroke and syringe pressure perspective for various clinical situations. When the return stroke of the syringe contains only blood, the syringe return rate, and the pressure curve will have a specific profile. In the event that there is clot mixed in with the blood, as the clot passes through the known restriction in the path between the syringe and the clot filter, the stroke rate may be reduced and/or the pressure increase in a non-uniform way, which depending on the chronicity and size of the clot, will have a unique pressure profile and syringe return stroke profile. Through testing each unique clot size and clot types can be characterized through the restrictor, thus allowing the system to be able to quantity and qualify the clot that is aspirated into the system. This information can be vital to the user to better understand the patients' disease(s), symptoms, and level of care that may be required. Additionally, this data can be collected on a large number of patients to better understand population-wide trends or correlations to clot types and locations to better information the doctors and healthcare industry.

Also described herein are methods and apparatuses for error state detection. If the system encounters errors due to mechanical and/or electronic component failures which may impact the system's ability to perform, the various input signals such as the pressure and syringe/actuator position can be used to understand when the system is in an error state, by seeing signals outside of the acceptable range of values. In the case that there is an error with the syringe not being able to fully retract during an aspiration, for example, the encoder data will show the system that the resting “back” position is not within the acceptable range, and the system can then inform the user of a fault. In some cases, these faults may be recoverable through user intervention, but may also not be recoverable, which would inform the user to replace the malfunctioning parts. This is a benefit to patient care and safety as it can ensure only quality products that are within the performance specifications are being used, ensuring a more repeatable and effective patient treatment.

FIG. 51 shows another example of a system 5100 that may be configured to include a smart fluidic drive system. In FIG. 51, the system includes an aspiration piston (syringe 5161), a pneumatic actuator 5162 driving the aspiration piston, pneumatic regulators 5163a, b, flow control valves 5165a, b/pneumatic variable flow restrictors 5175, pneumatic valves 5166, a pressure source 5168, one or more pneumatic pressure transducers 5169, 5169′, and electronics 5170. This example also shows a position encoder 5171 for determining the position of the piston/syringe. The electronics 5170 may include software, hardware and/or firmware for receiving inputs and controlling outputs of any of these elements, as discussed in detail above.

### EXAMPLE

FIGS. 52A-52D and 53A-53C illustrate one example of a closed-loop clot removal and blood return apparatus (e.g., system) as described herein. FIG. 52A shows an aspiration catheter 5261 that may optionally be included as part of the apparatus. In this example the aspiration catheter includes an integrated navigation catheter 5263 that may be used for steering/guidance. An aspiration opening is at the distal end region 5264 of the catheter and includes a pair of electrodes for clot/blood/wall sensing. The catheter also includes a handle region 5262 that may also include one or more controls, shown in FIG. 52D, such as a rotation control (knob 5276) (for rotating the aspiration catheter relative to the handle) and a button 5271 for activating a burst or pulse of suction. The handle may also include an accessory port and a flush port.

FIG. 52B shows an example of an integrated aspiration/pumping system (sub-system) that may be used, similar to the example shown in FIGS. 43-44. In FIG. 52B the aspiration/pumping sub-system 5265 includes a pneumatically driven piston (e.g., syringe) that may be used to apply negative pressure (aspiration) to the connected aspiration catheter (e.g., FIG. 52A) during a first stroke and may apply positive pressure to a second part of the blood removal and return circuit. As described above, one or more one-way valves may prevent aspiration in the second part of the blood removal circuit and positive pressure from the aspiration catheter. In this example, the sub-system may include a controller that may control the pneumatic operation of the piston (e.g., syringe) so that different pressures, and therefore different flow rates, may be applied for aspiration (during the first stroke) and positive pressure (during the second stroke). The aspiration portion of the pumping cycle may remove clot and blood into the tubing 5273 connecting the aspiration catheter to the aspiration/pumping sub-system 5265 and may be driven at a relatively higher rate (higher flow rate) than the positive pressure portion of the pumping cycle, which may be used to drive blood return into the body. The aspiration/pumping sub-system 5265 also includes an indicator (e.g., LED) for indicating when the sensors, e.g., at the aspiration catheter distal end region) indicate that the catheter is in contact with clot, blood, or vessel wall.

In FIG. 52B the aspiration/pumping sub-system 5265 includes an integrated powered syringe pump that is pneumatically driven by an integrated source of stored, compressed fluid (e.g., CO2 cannister) 5268, and a battery pack. The aspiration/pumping sub-system 5265 also include a clot collection chamber 5267 and a deaeration chamber 5269. The output of the deaeration chamber couples to a blood return line 5272 including a second filter 5275 (e.g., a 40 micron filter) for returning the blood to the patient.

The aspiration/pumping sub-system also include one or more controls, such as an aspiration control 5271 that may be triggered to apply aspiration (in this example, redundant to the control on the handle), and a volume control 5269 for selecting the volume of blood/clot material to be aspirated (e.g., 60 cc, 30 cc, 15 cc, 10 cc, etc.).

FIGS. 53A-53C show additional details on the clot collection chamber 5267, which includes blood filtration, and a clot strainer 5278 (FIG. 53B), over a filter 5279 (e.g., 150 micron filter, shown in FIG. 53C). In FIG. 53A the clot collection chamber 5267 shown with the viewing window 5277 forming a cover that is screwed down (and sealed over) the clot collection chamber. The viewing window includes a wiper control 5281 for moving a wiper within the chamber to wipe the viewing window inside of the chamber. In FIG. 53B the viewing window 5277 is removed, showing the clot strainer 5278. In FIG. 53C the clot strainer is removed, showing the underlying filter 5279. The outlet port for the clot collection chamber is below the filter (not shown).

FIGS. 54A-54I illustrate one example of a use of the apparatus shown in FIGS. 52A-52D and 53A-53C. In FIG. 54A, the catheter 5261 is inserted, e.g., over a wire, and/or over the navigation catheter integrated into the device. In the presence of blood, the LED 5277 is green, as shown.

As shown in FIGS. 54B-54C, once the catheter tip 5263 contacts clot material 5290, the LED 5277 turns orange. If the catheter tip disengages from clot, the lights will flash orange and then return to green. If the tip contacts wall, the indicator will turn blue. Other indicators or indicator colors may be used.

Clot material 5290 may be removed by triggering a pulse of aspiration, e.g., by pushing the button 5271′ on the handle of the aspiration catheter, as shown in FIG. 54D, causing the piston to be driven proximally and aspirating clot material and blood into the aspiration tubing 4709, as shown. Additional pulses of aspiration may be applied, and more clot material may be aspirated. After each aspiration pulse, the system may re-advance the piston/syringe (distally) and may drive any blood and clot material within the circuit (and in particular in the chamber of the piston/syringe) into the clot collection chamber 5267 using positive pressure from the second stroke of the pump. As described above, the clot material may be captured and filtered out of the blood in the clot collection chamber 5267, as shown in FIG. 54E. The filtered blood may then be moved to the deaeration chamber 5269 to remove air bubbles (as shown in FIG. 54F) before blood is returned to the patient. The distal tip may be navigated to different regions, including bifurcations, by advancing and withdrawing over the navigation catheter and by rotating the catheter, e.g., to switch between the right and left pulmonary arteries. The tip and shaft of the catheter may be rotated independently.

FIGS. 54F-54G show the filtered blood within the blood return line 5272. The blood may be filtered again (e.g., using a secondary filter 5275) before being reintroduced into the patient (shown as a patient model 5491 in FIG. 54H). The removed clot material 5290 may be visualized through the window of the clot collection chamber or may be removed from the clot collection chamber as shown in FIGS. 53A-53C and in FIG. 54I.

### Catheter

The methods and apparatuses described herein may be used with any appropriate aspiration catheter, including (but not limited to) those shown and described in U.S. Pat. Nos. 11,730,924, 12,246,141, 11,730,925, 12,274,834, U.S. patent application Ser. No. 18/329,535, U.S. patent application Ser. No. 18/671,981, U.S. patent application Ser. No. 18/935,426, U.S. patent application Ser. No. 19/192,303, U.S. patent application Ser. No. 18/859,162, and U.S. patent application Ser. No. 18/665,380. For example, FIGS. 55A-55E and 56A-56B illustrate examples of catheters that may be used with any of the methods and apparatuses described herein.

For example, in FIG. 55A the distal end region (e.g. tip region 5502) may include side-facing (angled) aspiration opening 5504 into the aspiration lumen 5522 of the aspiration catheter. The tip may be continuous with the long length of the aspiration catheter or it may be coupled to the distal end of the catheter so that the aspiration opening is in fluid communication with the aspiration opening. The aspiration catheter tip 5502 in this example is also configured to hold a navigation catheter and/or guidewire 5506 that may be extended through the aspiration catheter to exit from an opening that is distal to the aspiration opening 5504. In this example the distal end region tapers to form distal end opening (e.g., eyelet or tip eyelet 5508) for passing the navigation catheter and/or guidewire. In some cases the cyclet region may be configured to pass a navigation catheter (which may be configured to hold and/or pass a guidewire therethrough) or directly pass a guidewire. In FIG. 55A the side of the distal tip region is shown as transparent, allowing visualization of the navigation catheter 5506 extending within the aspiration lumen 5522. In any of these catheters the eyelet region may be in fluid communication with a separate navigation catheter and/or guidewire channel (not shown) that may be within or adjacent to the aspiration lumen. A navigation catheter and/or guidewire channel may be open to the aspiration lumen in some regions or entirely enclosed. In some cases the navigation catheter and/or guidewire channel may extend just partially through the aspiration catheter (e.g., through the distal 1 cm, 2 cm, 5 cm, 10 cm, 15 cm, 20 cm, etc. or less). In some cases the navigation catheter and/or guidewire channel may extend through the full length of the aspiration catheter.

In general the aspiration catheters described and illustrated herein (also referred to herein as thrombectomy catheters) may have enhanced trackability, clot removal efficiency, and user ergonomics. For example, any of these apparatuses may include a reinforced aspiration tip, as shown in FIGS. 55A-55E. In some cases, these tips may include one or more integrated sensing electrodes, and/or a trifilar wire.

The aspiration catheters may have any appropriate catheter shaft diameter (inner and/or outer), particularly catheter shaft configurations between about 12F and 30F (e.g., between 14F-28F, between 16F-24F, etc.). Any of these catheters may include a modular handle that is configured to include or be used with automated fluid control. These modifications may prevent or limit vessel wall latching, blood loss, and procedural complexity.

As mentioned, any of these aspiration catheters may be used with a navigation catheter and/or guidewire and may include a port or channel for a navigation catheter and/or guidewire, which may be within the lumen (e.g., within or adjacent to the aspiration lumen of the catheter), through a wall region of the catheter and/or coupled to the wall of the catheter.

In some examples the interface between the navigation catheter and/or guidewire may be configured as an eyelet or opening 5508. In FIGS. 55A-55E, the eyelet for the navigation catheter/guidewire may be configured such that the inner diameter of the eyelet region is narrower than the navigation catheter/guidewire lumen. Thus the distal end opening forming the eyelet may be configured so that the eyelet fits the navigation catheter/guidewire more snugly (e.g., “hugs” the navigation catheter/guidewire), by constricting down over the navigation catheter/guidewire just at the distal end region (in some cases, the distal tip). For example, the inner diameter of the narrow (e.g., 5 mm or less, 4 mm or less, 3 mm or less, 2 mm or less, 1 mm or less, 0.8 mm or less, 0.7 mm or less, 0.6 mm or less, 0.5 mm or less, 0.4 mm, 0.2 mm or less, etc.) eyelet region may have an inner diameter that is less than the inner diameter (ID) of the rest of the navigation catheter/guidewire lumen. For example, the ID of the eyelet region may be 95% of the ID of the rest of the navigation catheter/guidewire lumen, if present, and/or the ID of the aspiration lumen (e.g., 90%, 85%, 80%, 75%, 70%, 65%, 60%, 55%, 50%, 45%, 40%, 35%, 30%, 25%, 20%, 15%, etc. or less). The ID of the eyelet region may be configured to be very close to the outer diameter of the navigation catheter/guidewire, while the rest of the navigation catheter lumen may be slightly oversized by comparison.

The narrower ID of the eyelet region of the navigation catheter/guidewire lumen opening may enable the tip of the aspiration catheter shaft to track more closely to the navigation catheter while maintaining independence proximally of the tips region. This may also reduce friction and improve torquability in tortuous anatomy. This configuration may allow the navigation catheter/guidewire to be removed from the aspiration catheter to allow for a larger internal cross sectional area from which to extract clot.

FIGS. 55B and 55C illustrate examples of aspiration catheter tip regions having an aspiration opening 5504 with a reinforced lip 5512. In this example the reinforced lip 5512 is formed by regions of different thickness that extend from a top rim region 5520, which is cut into the wall of the aspiration catheter and extends transverse to the long axis of the tip. In this example the top rim region 5520 extends partially around the radius of the aspiration catheter (in an un-tapered region) between about 5 degrees and 50 degrees (e.g., between about 5-25 degrees, etc.). This is shown in better detail in FIG. 55D. The aspiration opening is formed at a tapered angle (e.g., between about 10 degrees and 60 degrees, between 10 degrees and 50 degrees, between 15 degrees and 45 degrees, etc.), as shown. In this example, the taper forming the aspiration opening is on just one side of the catheter, so that the opposite side remains in-line with the outer diameter of the aspiration catheter.

The thickened rim in this example may be formed by adding additional polymeric material (e.g., thicker polymer) and/or by including one or more reinforcement structures (e.g., wires, coils, etc.), such as a nitinol micro coil. In some cases the reinforced rim may be reinforced by using a stiffer material, such as a stiffer polymer (e.g., higher durometer polymer) as compared with the rest of the tip region and/or catheter body. FIG. 55C shows a reinforced lip 5512 that includes a thicker rim 5518 that has a different thickness over the region between the top rim region 5520 and a more distal midpoint region 5530, 5530′. The region distal to the midpoint region 5530, 5530′ region may be less thickened and therefore less reinforced. This may allow a more controlled collapsing at this midpoint region or distal to the midpoint region. This configuration may therefore include a thinner proximal edge 5532, which may be used as a cutting edge. Thus, in addition to thickening the lateral sides of the aspiration orifice, the proximal edge may be locally thinned to enhance clot shearing during aspiration.

Any of these aspiration catheters may include one or more holes or openings (e.g., clot-fluid optimization holes, CFOs) that may be configured to help prevent or reduce latching, which may occur when the aspiration opening sucks onto the wall, instead of, or in addition to, clot material. In FIGS. 55A-55E, the tip 5502 includes a pair of openings 5528, 5528′ (CFOs) opposite from the aspiration opening on the straight (non-tapered) region of the catheter (catheter tip) that is distal to the midline (e.g., the midpoint region 5530, 5530′) of the aspiration opening. This is shown in greater detail in FIG. 55E. The aspiration openings 5528, 5528′ may underlie the region of the aspiration lumen where the navigation catheter/guidewire may pass (in this example, unconstrained), and opposite the distal portion of the aspiration opening 5504. This arrangement may allow release of inadvertently aspirated vessel walls, while maintaining vacuum efficiency during clot extraction due to the clot being pulled into the tip proximal to the hole locations (e.g., proximal to the midline. In FIG. 55E the PFOs are positioned in the distal half (distal most 50%, 45%, 40%, 35%, 30%, etc.) of the region opposite the aspiration opening.

Any of these aspiration catheters may include one or more sensors, e.g., for sensing clot. For example, the aspiration catheter tip 5502 may include one or more sensors, including one or more monopolar electrodes and/or one or more electrode pairs. In some cases the sensor may be an electrical sensor that may detect contact and/or proximity to a material, such as clot material, wall, blood, etc. based on an electrical property sensed using the electrode(s). For example, the catheter may include one or more electrodes and/or electrode pairs for sensing an electrical property such as impedance, resistance, capacitance, etc.

In some cases it may be beneficial to include one or more electrodes that are integrated with the distal tip. For example, the one or more electrodes may be integrated outside of the aspiration lumen, e.g., on the lip of the aspiration opening and/or adjacent to the lip. In some cases one or more electrodes and/or electrode pairs (or one electrode of an electrode pair) may be distal to the aspiration opening.

FIGS. 55B-5D illustrate integrated electrodes 5514, 5514′ on the lip or rim of the aspiration opening 5504. In this example, the integrated electrodes 5514, 5514′ are micromachined electrodes that are embedded in the tip and connected to a wire (e.g., a trifler wire). These electrodes could be manually inserted into the tip or overmolded in position to retain the wire and electrodes. For example, in FIG. 55D the electrodes 5514, 5514′ are shown embedded in the thicker rim region, on the proximal half of the aspiration opening, adjacent to the cutting edge.

As mentioned, the aspiration catheter may include any appropriate length and diameter (e.g., in some cases between 12F and 30F, such as 16F, 18F, 20F, 24F, etc.). In one example the aspiration catheter may be a 24F catheter that is reinforced in regions along its length to permit torque and resist collapse. For example, a catheter, including the 24F catheter, may include a shaft having multiple outer diameters (ODs) along its length; in some cases it may have a larger OD on the distal end region, and this region may include a reinforcing coil and/or a braid to enhance torque transmission and to resist collapse. In some cases the proximal end of the aspiration catheter may only include a braid to increase stiffness relative to the more flexible distal end, as this proximal stiffer section may not be inserted into the regions having greater tortuosity (curvature), which may otherwise lead to shaft kinking or collapse.

In any of these apparatuses, including a 24F catheter, the distal region may have a layered construction, such a polymeric (e.g., Pebax®) liner, a braid (e.g., stainless steel braid), a coil (e.g., a stainless steel coil) and a variable-durometer outer jacket. As mentioned, the outer diameter of this region may be, e.g., 24F. In any of these apparatuses the proximal segment (proximal region) may have a smaller OD. For example, the proximal region may not include the coil from the distal segment, which may reduce the OD (e.g., from 24 to 22F) over this region, enabling blood return between proximal end of the catheter and an introducer sheath.

Any of these apparatuses may include a flat wire lumen that may be embedded between the liner and the braid, and may be lubricious (e.g., may be coated with a lubricious material and/or formed of a lubricious material, such as a PTFE-coated material). This region may house the trifilar wire forming the electrical connections to the electrodes on the distal end region (e.g., tip region). The flat wire lumen and/or wires may terminate proximally, as shown in FIG. 56A in a slack chamber to accommodate wire elongation/compression during bending.

In some examples, e.g., a 16F example, the aspiration catheter may include a more uniform outer diameter along the length of the catheter. For example, the aspiration catheter may have a 16D OD along the length and may include just a braided shaft (without a coil).

The catheter may include a slanted tip configuration, e.g., having an approximately 45° angled opening (in some cases with a preformed curve of an about 25° angle in the catheter distal end region). This configuration may enhance vessel sweeping and directional aspiration using the tip.

The electrodes may each be electrically coupled to a wire (e.g., connector) such as, but not limited to, a multifilament (e.g., 2 filaments, 3 filaments, 4 filaments, 5 filaments, etc.) that are twisted or wound together). In some cases the wires used to connect the electrodes to the sensing electronics may be trifilar wires. The trifilar wire may include one or more outer wires (e.g., copper wires, which may connect to an electrode) and one or more central wires (e.g., stainless steel wires) that may reduce capacitance and/or may enhance pushability

The connecting wires may be routed through the catheter, e.g., through a wire lumen of the catheter that is within the aspiration lumen and/or separate from the aspiration lumen. In some cases the wire lumen may be a rectangular lumen, which may be sealed via a slack chamber to prevent vacuum loss. For example, FIG. 56A shows a portion of a handle region of an aspiration catheter, showing the slack chamber and a slack fin 5625 within the slack chamber that is part of the vacuum field. This slack fit may assist in managing the wires even during tortious movement of the catheter.

In any of these apparatuses the handle of the aspiration catheter may be configured to permit roll of the catheter relative to the handle. This roll may be infinite—e.g., may not require reversing between clockwise and counterclockwise, but may allow unlimited roll in either clockwise or counterclockwise. In FIG. 56A-56B the handle assembly 5600 includes a rotating shaft mechanism that allows the catheter to rotate independently via a polycarbonate hub with one or more conductive rings 5627. This configuration allows for an unlimited number of rotations of the catheter, as brushes on the assembly (e.g., on a printed circuit board, PCB, 5529) may maintain stable electrical contact during rotation and when not rotating.

The handle may also include one or more controls and/or grip(s). For example the handle may include a rotational (finger) grip region to rotate the catheter relative to the rest of the handle. Any of these handles may include a trigger, e.g., an aspiration trigger for controlling the application of aspiration through the catheter manually (a separate automatic control may also or alternatively be used). Any of these controls may be buttons, switches, dials, etc. For example, the aspiration trigger may be configured as a button-activated trigger that is integrated with the PCBA for impedance feedback.

The handle may include one or more coupling regions for coupling to a controller, a source of suction, etc. In some cases the handle may include one or more seals for making the connection, such as a trap door seal that automatically opens upon connection to aspiration source, eliminating or reducing manual fluid control. The handle may include one or more additional ports, such as an accessory port. The accessory port may accept devices up to 12F (e.g., guidewires, navigation catheter, etc.); the accessory port may be a hemostatic port or may couple to a hemostatic port. In some cases the accessory port may be in fluid communication with the aspiration lumen and/or with a navigation catheter/guidewire lumen.

Any of these apparatuses may also or additionally include one or more fluid/pressure ports. The fluid/pressure port may be configured to allow injection of contrast and/or pulmonary artery pressure monitoring.

In general, the apparatuses described herein may provide improved safety as compared to other catheters. For example, the CFO holes may reduce vessel wall aspiration risk, and electrodes may enable real-time tissue differentiation. These catheters may also provide advantages in efficiency, as reinforced tips may maintain aspiration integrity. The use of multifilament (e.g., trifilar) wire to connect to electrical sensors may minimize signal interference.

These handles may provide improved and/or enhanced ergonomics. For example, the rotating shaft may prevent wire tangling. The trap door seals may simplify workflow.

Finally, and somewhat unexpectedly, the ability to aspirate clot and break it off the wall/from a larger piece of clot may be enabled by the handle rotation and the directional aspiration. For example, a method of operation to remove adherent clot may include latching onto the clot and/or wall and rotating the entire catheter so that the tip rotates (e.g., between 1-90 degrees or more) while at least initially adherent to the clot (e.g., clot on the wall). In variations having PFOs, the aspiration opening may, after a delay (e.g., of 0.5-5 seconds) automatically release from the wall and/or clot, allowing repositioning of the tip (e.g., rotating back to the initial position) and/or aspiration of the released clot. This latching/rotating/releasing may be repeated multiple times to remove clot material. The aspiration may be turned off (e.g., when repositioning) and/or may remain on during the procedure. All or part of the procedure may be performed while imaging (e.g., using fluoroscopy) and/or while sensing (e.g., electrically sensing) clot, blood and/or wall at the distal end region of the tip.

The present disclosure contains the following Clauses:

Clause 1. An aspiration device for use with (a) an aspiration catheter and (b) a fluidic actuator configured to deliver a pressurized drive fluid, said aspiration device comprising: an aspirator including an aspirator displacement element, an aspirator cylinder, and an aspirator port, wherein the aspirator port is configured to connect to an aspiration lumen of the aspiration catheter; and a fluidic driver including a driver displacement element, a driver cylinder, and at least a first fluid port configured to receive the pressurized drive fluid from the fluidic actuator, wherein the pressurized fluid causes the driver displacement element to translate in a first direction and wherein the aspirator displacement element is coupled to travel in tandem in the first direction with the driver displacement element to draw blood and clot at least partially through the aspirator port and into and from the aspiration cylinder, when the aspiration catheter is in a patient blood vessel proximate clot.

Clause 2. The aspiration device of Clause 1, wherein the fluidic aspirator is configured to deliver at least a positive pressure drive fluid.

Clause 3. The aspiration device of Clause 1, wherein the fluidic aspirator is configured to deliver at least a negative pressure drive fluid.

Clause 4. The aspiration device of Clause 1, wherein the fluidic aspirator is configured to deliver both a positive pressure drive fluid and a negative pressure drive fluid.

Clause 5. The aspiration device of Clause 1 to 4, wherein the fluidic driver further includes a second fluid port configured to receive the pressurized drive fluid from the fluidic actuator, wherein the pressurized fluid causes the driver displacement element to translate in a second direction and wherein the aspirator displacement element is coupled to travel in tandem in the second direction with the driver displacement element eject blood and clot from the aspirator port.

Clause 6. The aspiration device of Clause 1 to 4, wherein the fluidic driver further includes a biasing spring coupled to one side of the driver displaceable element, wherein said biasing spring is configured to counteract translation of the displacement element caused by the pressurized fluid.

Clause 7. The aspiration device of Clause 1 to 6, wherein the aspirator and the fluidic driver are arranged in parallel.

Clause 8. The aspiration device of Clause 1 to 7, wherein the aspirator and the fluidic driver are arranged in tandem.

Clause 9. The aspiration device of Clause 1 to 7, wherein the aspirator and the fluidic driver are disposed in a common housing.

Clause 10. The aspiration device of Clause 9, wherein the common housing comprises a cylinder having an internal wall separating the aspirator and fluidic driver.

Clause 11. The aspiration device of Clause 1 to 9, wherein the aspirator and the fluidic driver are disposed in separate housings.

Clause 12. The aspiration device of Clause 11, wherein the driver displacement element and the aspiration displacement element are joined by a coupling member disposed between the separate housings.

Clause 13. The aspiration device of Clause 1 to 12, wherein at least one of the displacement elements of the aspirator and the fluidic driver comprises a piston.

Clause 14. The aspiration device of Clause 1 to 13, wherein the displacement elements of the aspirator and the fluidic driver each comprise a piston.

Clause 15. The aspiration device of Clause 14, wherein the pistons are configured to reciprocate in their respective cylinders with low friction.

Clause 16. The aspiration device of Clause 1 to 12, wherein at least one of the displacement elements of the aspirator and the fluidic driver comprises a diaphragm.

Clause 17. The aspiration device of Clause 1 to 12 and 16, wherein the displacement elements of the aspirator and the fluidic driver each comprise a diaphragm.

Clause 18. The aspiration device of Clause 1 to 17, wherein the aspirator comprises a syringe.

Clause 19. The aspiration device of Clause 1 to 18, further comprising the fluidic actuator.

Clause 20. The aspiration device of Clause 19, the fluidic actuator comprises an aspiration controller.

Clause 21. The aspiration device of Clause 20, wherein the aspiration controller is programmable.

Clause 22. The aspiration device of Clause 21, wherein the aspiration controller is configured to respond to real-time user input.

Clause 23. The aspiration device of Clause 20 to 22, wherein the aspiration controller is configured to modify a length of travel of the driver displacement element to control one or more of flow volume and flow rate.

Clause 24. The aspiration device of Clause 20 to 23, wherein the aspiration controller is configured to modify a reciprocation rate of the driver displacement element to control one or more of flow volume and flow rate.

Clause 25. The aspiration device of Clause 20 to 24, wherein the aspiration controller is configured to receive data from one or more sensors which measure pressure, electrical impedance, optical properties, flow rate, and/or flow volume.

Clause 26. The aspiration device of Clause 25, wherein the aspiration controller is configured to control one or more of length of travel and reciprocation rate of the driver displacement element to control one or more of flow volume and flow rate in response to the received data.

Clause 27. The aspiration device of Clause 1 to 26, wherein the fluidic driver and fluidic actuator comprise a pneumatic driver and a pneumatic actuator.

Clause 28. The aspiration device of Clause 1 to 26, wherein the fluidic driver and fluidic actuator comprise a hydraulic driver and a hydraulic actuator.

Clause 29. An aspiration device for use with (a) an aspiration catheter and (b) a fluidic actuator configured to deliver a pressurized drive fluid, said aspiration device comprising: an aspirator including an aspirator displacement element, an aspirator cylinder, and an aspirator port, wherein the aspirator port is configured to connect to an aspiration lumen of the aspiration catheter; and a fluidic driver including a driver displacement element; and a coupling element configured to drive the aspirator displacement element in tandem in with the driver displacement element to draw portions of blood and clot through the aspirator port and into and from the aspiration cylinder, when a distal port of the aspiration catheter is in a patient blood vessel proximate clot; wherein a travel distance of the coupling element is adjustable to control the volume of blood and clot portions aspirated into the aspiration catheter.

Clause 30. The aspiration device of Clause 29, further comprising travel stops that limit the travel of the coupling element.

Clause 31. The aspiration device of Clause 29, wherein the travel stops comprise pins and ledges controlled by a knob.

Clause 32. A fluidic actuator configured to deliver a pressurized drive fluid to a fluidic driver coupled to an aspirator and an aspiration catheter, said fluidic actuator comprising; a source of pressurized fluid; a valve arrangement for selectively delivering the pressurized fluid from the pressurized fluid source to a first fluid port of the fluidic driver, wherein the fluidic driver includes a driver displacement element and a driver cylinder, wherein the driver displacement element is coupled to an aspirator displacement element of the aspirator, and wherein delivery of the pressurized fluid to the first fluid port causes the driver displacement element to move the aspirator displacement element in a first direction to draw blood and clot through an aspirator port and delivery of the pressurized fluid to the second fluid port causes the driver displacement element to move the aspirator displacement element in a second direction to eject blood and clot through the aspirator port, respectively, when the aspiration catheter is in a patient blood vessel proximate clot.

Clause 33. The fluidic actuator of Clause 32, the fluidic actuator comprises an aspiration controller.

Clause 34. The fluidic actuator of Clause 33, wherein the aspiration controller is programmable.

Clause 35. The fluidic actuator of Clause 34, wherein the aspiration controller is configured to respond to real-time user input.

Clause 36. The fluidic actuator of Clause 33, wherein the aspiration controller is configured to modify a length of travel of the driver displacement element to control one or more of flow volume and flow rate.

Clause 37. The fluidic actuator of Clause 33, wherein the aspiration controller is configured to modify a reciprocation rate of the driver displacement element to control one or more of flow volume and flow rate.

Clause 38. The fluidic actuator of Clause 33, wherein the aspiration controller is configured to receive data from one or more sensors which measure pressure, electrical impedance, optical properties, flow rate, and/or flow volume.

Clause 39. The fluidic actuator of Clause 33, wherein the aspiration controller is configured to control one or more of length of travel and reciprocation rate of the driver displacement element to control one or more of flow volume and flow rate in response to the received data.

Clause 40. A method of clot removal and blood return comprising: positioning an aspiration catheter adjacent to a first clot material within a patient; actuating a pressure source one or more times, wherein each actuation of the pressure source: aspirates the first clot material and blood, filters the first clot material from the blood, and returns filtered blood to the patient; repositioning the aspiration catheter adjacent to a second clot material within the patient; and actuating the pressure source one or more times, wherein each actuation of the pressure source: aspirates the second clot material and blood, filters the clot material from blood, and returns filtered blood to the patient.

Clause 41. The method of Clause 40, wherein each actuation comprises a first stroke and a second stroke.

Clause 42. The method of Clause 41, wherein the first stroke comprises a first movement of a piston of the pressure source and wherein the second stroke comprises a return movement of the piston of the pressure source.

Clause 43. The method of Clause 41, wherein the first stroke comprises a negative pressure stroke and the second stroke comprises a positive pressure stroke.

Clause 44. The method of Clause 41, wherein the first stroke results in a first flow rate by which clot material is aspirated with blood and wherein the second stroke results in a second flow rate by which filtered blood is returned to the patient.

Clause 45. The method of Clause 44, wherein the first flow rate is greater than the second flow rate.

Clause 46. The method of Clause 40, further comprising sensing that the aspiration catheter is adjacent to the first clot material using one or more sensors on the aspiration catheter.

Clause 47. The method of Clause 46, wherein sensing comprises sensing an electrical signal.

Clause 48. The method of Clause 40, wherein each actuation of the pressure source comprises operating a control in communication with the pressure source.

Clause 49. The method of Clause 47, wherein operating the control comprises pushing a button.

Clause 50. The method of Clause 40, wherein actuating the pressure source comprises (a) aspirating blooding and clot from the aspiration catheter and (b) collecting filtered blood in a reservoir in one step and returning filtered blood from the reservoir to the patient in another step.

Clause 51. The method of Clause 50, wherein aspirating and filtering the blood are performed at a first flow rate and returning the filtered blood to the patient is performed at a second flow rate slower than the first flow rate.

Clause 52. The method of Clause 40, wherein actuating the pressure source is automatically actuated by a controller.

Clause 53. The method of Clause 52, wherein the controller is programmed to both (a) aspirate blood and clot from the aspiration catheter and (b) collect filtered blood in a reservoir in one step and to return filtered blood from the reservoir to the patient in another step.

Clause 54. The method of Clause 53, wherein the controller is further programmed to aspirate and filter the blood at a first flow rate and return the filtered blood to the patient at a second flow rate slower than the first flow rate.

Clause 55. A method of clot removal and blood return comprising: positioning an aspiration catheter adjacent to a first clot material within a patient; actuating a control to trigger a pressure source, wherein each actuation causes the pressure source to: aspirate the first clot material, filter the first clot material from blood, and return filtered blood to the patient; repositioning the aspiration catheter to be adjacent to a second clot material within the patient; and actuating the control to trigger the pressure source, wherein each actuation causes the pressure source to: aspirate the second clot material, filter the second clot material from blood, and return the filtered blood to the patient.

Clause 56. The method of Clause 55, wherein each actuation causes the pressure source to deliver a first stroke and a second stroke.

Clause 57. The method of Clause 56, wherein the first stroke comprises a negative pressure stroke and the second stroke comprises a positive pressure stroke.

Clause 58. The method of Clause 56, wherein the first stroke results in a first flow rate by which clot material is aspirated with blood and wherein the second stroke results in a second flow rate by which filtered blood is returned to the patient.

Clause 59. The method of Clause 58, wherein the first flow rate is greater than the second flow rate.

Clause 60. The method of Clause 55, further comprising sensing, using one or more sensors on the aspiration catheter, that the aspiration catheter is adjacent to the first clot material.

Clause 61. The method of Clause 60, wherein sensing comprises sensing an electrical impedance.

Clause 62. The method of Clause 55, wherein actuating the control comprises pushing a button.

Clause 63. A method of clot removal and blood return comprising: positioning an aspiration catheter adjacent to a first clot material within a patient; actuating a control to trigger a pressure source, wherein each actuation causes the pressure source to generate a negative pressure stroke that aspirates the first clot material and a positive pressure stroke that returns filtered blood to the patient, wherein the blood is filtered during the negative pressure stroke and/or the positive pressure stroke; repositioning the aspiration catheter to be adjacent to a second clot material within the patient; and actuating the control to trigger the pressure source, wherein each actuation causes the pressure source to generate the negative pressure stroke that aspirates the second clot material and the positive pressure stroke that returns filtered blood to the patient, wherein the blood is filtered during the negative pressure stroke and/or the positive pressure stroke.

Clause 64. The method of Clause 63, wherein the negative pressure stroke results in a first flow rate by which clot material is aspirated with blood and wherein the positive pressure stroke results in a second flow rate by which filtered blood is returned to the patient.

Clause 65. The method of Clause 64, wherein the first flow rate is greater than the second flow rate.

Clause 66. The method of Clause 65, further comprising sensing, using one or more sensors on the aspiration catheter, that the aspiration catheter is adjacent to the first clot material.

Clause 67. The method of Clause 66, wherein sensing comprises sensing an electrical impedance.

Clause 68. A method, the method comprising: positioning an aspiration catheter proximate a clot material; applying a first pressure from one or more pumps to pull the clot material and blood through the aspiration catheter and into a fluid circuit at a first flow rate; applying a second pressure from the one or more pumps to push the blood back into the body from the fluid circuit at a second flow rate after the clot material has been filtered from the blood; and independently controlling, using a controller coupled to the one or more pumps, a first flow rate and a second flow rate, wherein the first flow rate is different than the second flow rate.

Clause 69. The method of Clause 68, wherein the blood is pushed back into the body from a blood return portion of the fluid circuit that is pressurized to a return pressure by the second pressure, wherein the blood is pushed back into the body until the return pressure normalizes with a pressure within the body or until the blood within the blood return portion of the fluid circuit falls below a volume threshold.

Clause 70. The method of Clause 68, wherein the first flow rate is greater than the second flow rate.

Clause 71. The method of Clause 68, wherein the first pressure is less than 0 mmHg at the one or more pumps and the second pressure is greater than 0 mmHg at the one or more pumps.

Clause 72. The method of Clause 68, wherein the first pressure is a negative pressure and the second pressure is a positive pressure.

Clause 73. The method of Clause 68, wherein applying the first pressure and applying the second pressure comprises using the same pump to apply the first pressure and the second pressure.

Clause 74. The method of Clause 68 wherein applying the first pressure and applying the second pressure comprises using a first pump to apply the first pressure and using a second pump to apply the second pressure.

Clause 75. The method of Clause 68, wherein the one or more pumps comprises a piston pump.

Clause 76. The method of Clause 68, wherein the controller is configured to control the first flow rate and the second flow rate by controlling an application of a stored force to displace a piston within the one or more pumps.

Clause 77. The method of Clause 68, wherein applying the second pressure from the one or more pumps comprises pushing the clot material through the fluid circuit and back into the body.

Clause 78. A method, the method comprising: positioning an aspiration catheter proximate a clot material; applying a negative pressure from a pump to pull the clot material through the aspiration catheter and into a fluid circuit at a first flow rate; applying a positive pressure from a pump to push the blood back into the body from the fluid circuit at a second flow rate after the clot material has been filtered from the blood; and independently controlling, using a controller coupled to the pump, the first flow rate and the second flow rate, wherein the first flow rate is different than the second flow rate.

Clause 79. An apparatus, the apparatus comprising: one or more pumps configured to provide a first pressure and a second pressure, wherein the first pressure is negative and the second pressure is positive; a first inlet coupled to the one or more pumps and configured to couple in fluid communication to an aspiration catheter; a first outlet coupled to the one or more pumps and configured to fluidically couple the pump in fluid communication with a blood return line; and a controller coupled to the one or more pumps and configured to independently control the first pressure applied by the one or more pumps to the first inlet and the second pressure applied by the one or more pumps to the blood return line, wherein the first pressure is different than the second pressure.

Clause 80. The apparatus of Clause 79, wherein the one or more pumps comprises a single pump having a piston, wherein the controller controls the application of a third pressure to drive movement of the piston and thereby generate the first or second pressure.

Clause 81. The apparatus of Clause 79, further comprising the aspiration catheter coupled to the first inlet.

Clause 82. The apparatus of Clause 79, further comprising a de-airing chamber coupled to the blood return line.

Clause 83. The apparatus of Clause 79, further comprising one or more blood filters in fluid communication with the blood return line.

Clause 84. The apparatus of Clause 79, further comprising a first one-way valve in fluid communication with the first inlet and a second one-way valve in fluid communication with the first outlet, wherein the first one-way valve is oriented to allow blood to flow into a chamber of the pump from the aspiration catheter and the second one-way valve is oriented to allow blood to flow out the chamber of the pump into the blood return line.

Clause 85. The apparatus of Clause 79, further comprising a clot collection chamber configured to be fluidically connected between the first inlet and the aspiration catheter.

Clause 86. The apparatus of Clause 79, further comprising a clot collection chamber configured to be fluidically connected to the blood return line.

Clause 87. An apparatus, the apparatus comprising: a pump configured having a piston, wherein the pump is configured to provide a first pressure when driven in a first direction and a second pressure when driven in a second direction, wherein the first pressure is negative and the second pressure is positive; a first inlet coupled to the pump and configured to couple in fluid communication to an aspiration catheter so that the first pressure causes a first flow rate through the aspiration catheter; a first outlet coupled to the pump and configured to fluidically couple the pump in fluid communication with a blood return line so that the second pressure causes a second flow rate in the blood return line; and a controller coupled to the pump and configured to independently control the movement of the piston to generate the first pressure and the second pressure, wherein the controller is configured so that the first flow rate is greater than the second flow rate.

Clause 88. A method of closed-loop clot removal and blood return configured to withdrawal blood from a body at a rate that does not depend on the rate that blood is returned to the body, the method comprising: applying aspiration through a first portion of a blood removal and return circuit to draw a clot material and blood into the blood removal and return circuit at a first flow rate; and applying positive pressure through the blood removal and return circuit to drive the clot material and blood into a clot collection chamber within a second portion of the blood removal and return circuit, wherein the clot collection chamber comprises a capacitive air reservoir that is configured to hold a minimum volume of air between a filter and a visualization window of the clot collection chamber; filtering the blood within the clot collection chamber and passing the filtered blood into a second chamber; and returning blood from the second chamber to the patient at a second flow rate.

Clause 89. The method of Clause 88, further comprising compressing or expanding the capacitive air reservoir as blood passes through the clot collection chamber.

Clause 90. The method of Clause 88, further comprising allowing flow from the clot collection chamber to the second chamber but not from the second chamber to the clot collection chamber.

Clause 91. The method of Clause 88, further comprising removing the visualization window of the clot collection chamber to remove clot material from the clot collection chamber.

Clause 92. The method of Clause 91, wherein removing the visualization window comprises removing the visualization window without breaking the blood removal and return circuit.

Clause 93. The method of Clause 88, wherein the minimum volume comprises 10 cc or less.

Clause 94. The method of Clause 88, wherein the second flow rate is less than or equal to the first flow rate.

Clause 95. The method of Clause 88, wherein the second chamber has a second capacitive air reservoir that is configured to vent air from the second chamber.

Clause 96. The method of Clause 88, wherein the second chamber comprises a de-airing chamber.

Clause 97. The method of Clause 88, wherein the clot collection chamber is sealed to prevent air from outside of the clot collection chamber from entering the clot collection chamber.

Clause 98. The method of Clause 88, wherein the clot collection chamber comprises a wiper within the clot collection chamber configured to wipe the visualization window.

Clause 99. A closed-loop clot removal and blood return system, the system comprising: an aspiration line configured to fluidically couple to an aspiration catheter to remove clot and blood from a patient; a pressure source configured to apply aspiration through the aspiration line; a positive pressure lumen in fluid communication with the aspiration line; a clot collection chamber coupled to the positive pressure lumen and configured to receive the clot and blood from the patient, wherein the clot collection chamber comprises a viewing window and a capacitive air reservoir between with viewing window and a filter that is configured to filter the clot material from the blood, an inlet above the filter, and an outlet below the filter, wherein the capacitive air reservoir is configured to hold a minimum volume of air between the viewing window and the filter; a second chamber having a second inlet that is fluidically coupled to the outlet of the clot collection chamber and a second outlet that is lower than the second inlet; a one-way valve between the outlet and the second inlet, configured to allow flow from the clot collection chamber to the second chamber but not from the second chamber to the clot collection chamber; and a blood return line fluidically coupled to the second outlet.

Clause 100. The system of Clause 99, wherein the capacitive air reservoir is further configured to compress or expand as blood passes through the clot collection chamber.

Clause 101. The system of Clause 99, wherein the visualization window of the clot collection chamber is removable to allow clot material to be removed from the clot collection chamber.

Clause 102. The system of Clause 99, wherein the minimum volume comprises 10 cc or less.

Clause 103. The system of Clause 99, wherein the second chamber has a second capacitive air reservoir that is configured to vent air from the second chamber.

Clause 104. The system of Clause 99, wherein the second chamber comprises a de-airing chamber.

Clause 105. The system of Clause 99, wherein the clot collection chamber is sealed to prevent air from outside of the clot collection chamber from entering the clot collection chamber.

Clause 106. The system of Clause 99, wherein the clot collection chamber comprises a wiper within the clot collection chamber configured to wipe the visualization window.

Clause 107. The system of Clause 99, wherein the pressure source is configured to apply aspiration to the aspiration line at a first rate and to apply positive pressure to the positive pressure lumen at a second rate that is different from the first rate.

Clause 108. A method for clot aspiration, said method comprising: translating a positive displacement element in a chamber to draw blood and clot through a lumen of an aspiration catheter having a distal opening located in a patient's vasculature into a receiving volume of the chamber; monitoring pressure within the receiving volume of the chamber as the displacement element is being translated; controlling a rate of translating the positive displacement element to maintain a target pressure in the receiving volume at a target value or within a target range.

Clause 109. The method of Clause 108, wherein translating the positive displacement element in a chamber to draw blood and clot through the lumen of an aspiration catheter comprises powered retraction of a plunger in a chamber comprising a syringe barrel.

Clause 110. The method of Clause 109, wherein powered retraction a plunger in a syringe comprises fluidically or electrically powered retraction.

Clause 111. The method of Clause 108, wherein monitoring pressure within the receiving volume of the chamber comprises directly measuring the pressure with a pressure sensor located within the receiving volume.

Clause 112. The method of Clause 108, wherein monitoring pressure within the receiving volume of the chamber comprises indirectly measuring the pressure with a pressure or force sensor located externally of the receiving volume.

Clause 113. The method of Clause 108, wherein controlling the rate of translating the positive displacement element comprises maintaining a target pressure in the receiving volume in a target range.

Clause 114. The method of Clause 108, wherein the target pressure is maintained above a vacuum level that would cause hemolysis.

Clause 115. The method of Clause 108, wherein controlling the rate of translating the positive displacement element comprises retracting the positive displacement element in a series of steps to cause a pressure hammer effect.

Clause 116. A system for use with an aspiration catheter, said system comprising: a chamber; a positive displacement element translatably mounted in the chamber to draw blood and clot through a lumen of the aspiration catheter into a receiving volume of the chamber; a sensor configured to measure pressure within the receiving volume of the chamber as the displacement element is being translated; and a controller configured to receive an output of the sensor and to control a rate of translating the positive displacement element to maintain a target pressure in the receiving volume at a target value or within a target range.

Clause 117. The system of Clause 116, wherein the positive displacement element and the chamber comprise a plunger in a syringe assembly.

Clause 118. The system of Clause 116, further comprising a powered driver coupled to the positive displacement element and controlled by the controller.

Clause 119. The system of Clause 118, wherein powered driver comprises a fluidically powered driver.

Clause 120. The system of Clause 118, wherein powered driver comprises an electrically powered driver.

Clause 121. The system of Clause 116, wherein the sensor comprises a pressure sensor disposed within the receiving volume and configured to measure the pressure directly.

Clause 122. The system of Clause 116, wherein the sensor comprises a pressure or force sensor disposed externally of the receiving volume and configured to measure the pressure indirectly.

Clause 123. The system of Clause 116, wherein the controller is configured to control translation of the positive displacement element comprises at a rate selected to maintain a target pressure in the receiving volume in a target range.

Clause 124. The system of Clause 123, wherein the target pressure is maintained above a vacuum level that would cause hemolysis.

Clause 125. The system of Clause 116, wherein controlling the rate of translating the positive displacement element comprises retracting the positive displacement element in a series of steps to cause a pressure hammer effect.

Clause 126. A system for use with an aspiration catheter, said system comprising: a chamber; a positive displacement element translatably mounted in the chamber to draw blood and clot through a lumen of the aspiration catheter into a receiving volume of the chamber; a sensor configured to measure pressure within the receiving volume of the chamber as the displacement element is being translated; and a controller configured to receive an output of the sensor and to control a rate of translating the positive displacement element to maintain a target pressure in the receiving volume at a target value or within a target range.

Clause 127. A method for aspirating clot from the vasculature of a patient, said method comprising: positioning a distal port of an aspiration catheter proximate the clot in the patient's vasculature, wherein a proximal end of an aspiration lumen of the aspiration catheter is attached to an aspirator port of an aspirator including an aspirator displacement element and aspirator cylinder; and delivering a pressurized drive fluid to a first port of a fluidic driver comprising a driver displacement element and a driver cylinder, wherein the driver displacement element is coupled to the aspirator displacement element; wherein delivery of the pressurized fluid to the first port translates the driver displacement element and aspirator displacement element in a first direction which generates a negative pressure in the aspiration lumen to draw the clot and blood into the aspiration lumen through the distal port.

Clause 128. The method of Clause 127, wherein the pressurized fluid is delivered to the first port without interruption until a predetermined amount of clot has been collected in the aspirator cylinder.

Clause 129. The method of Clause 127, wherein delivery of the pressurized fluid to the first port is interrupted to vary a negative pressure in the aspiration lumen and at the port to enhance clot fragmentation and collection.

Clause 130. The method of Clause 127, further comprising delivering the pressurized fluid to a second port of the fluidic driver to translate the driver displacement element and aspirator displacement element in a second direction to eject the clot and blood through the aspirator port.

Clause 131. The method of Clause 130, further comprising diverting the clot and ejected through the aspirator port to a collection receptacle.

Clause 132. The method of Clause 130, further comprising diverting the clot and blood ejected through the aspirator port to a filter to separate blood from clot and returning the separated blood to the patent.

Clause 133. The method of Clause 130, wherein the pressurized fluid is delivered alternately to the first and second ports of the fluidic driver to oscillate a travel direction of the aspirator displacement element to cause a reversing flow pattern of clot and blood through the distal port of an aspiration catheter.

Clause 134. The method of Clause 130, wherein the pressurized drive fluid is delivered to the first and/or the second ports of the fluidic driver in a pattern which creates a pulsatile pressure profile at the distal port of the aspiration catheter to enhance clot fragmentation and collection.

Clause 135. The method of Clause 134, wherein the flow of pressurized fluid delivered to the first port is greater than the flow of pressurized fluid delivered to the second port so that there is a net accumulation of clot and blood in the aspirator cylinder.

Clause 136. The method of Clause 127, further comprising adjusting a translation length of the aspirator displacement element in the aspiration cylinder to vary a flow volume or rate of blood and clot into or from the distal port.

Clause 137. The method of Clause 127, further comprising adjusting a translation frequency of the aspirator displacement element in the aspiration cylinder to vary a flow volume or rate of blood and clot into or from the distal port.

Clause 138. The method of Clause 127, further comprising receive data from one or more sensors which measure pressure, electrical impedance, optical properties, flow rate, and/or flow volume.

Clause 139. The method of Clause 127, further comprising controlling one or more of length of travel and reciprocation rate of the driver cylinder to control one or more of flow volume and flow rate in response to the received data.

Clause 140. The method of Clause 127, wherein the pressurized fluid comprises a gas.

Clause 141. The method of Clause 127, wherein the pressurized fluid comprises a liquid.

Clause 142. A method of closed-loop clot removal and blood return using a blood removal and return circuit coupled to a patient, the method comprising: applying aspiration from an aspiration orifice by releasing stored positive pressure to generate on-demand negative pressure to draw a clot material from a blood vessel into the blood removal and return circuit; separating the clot material from blood by capturing the clot material into a chamber of the blood removal and return circuit; and returning filtered blood from the blood collection chamber to the patient.

Clause 143. A method of closed-loop clot removal and blood return using a blood removal and return circuit coupled to a patient, the method comprising: applying aspiration from an aspiration orifice by generating on-demand negative pressure using a stored positive pressure to displace a volume, to draw the clot material into the blood removal and return circuit; separating the clot material from blood by capturing the clot material in a chamber of the blood removal and return circuit; directing the blood into a blood collection chamber; and returning blood from the blood collection chamber to the patient.

Clause 144. A method of closed-loop clot removal and blood return using a blood removal and return circuit coupled to a patient, the method comprising: applying aspiration from an aspiration orifice by releasing stored positive pressure to drive a positive displacement piston and generate negative pressure to draw a clot material from a blood vessel into the blood removal and return circuit at a flow rate of greater than 0.1 L/min (e.g., greater than 0.5 L/min, greater than 1 L/min, greater than 1.5 L/min, 2 L/min, 2.5 L/min, 3 L/min, 3.5 L/min, 4 L/min, 4.5 L/min, 5 L/min, 5.5 L/min, 6 L/min, 6.5 L/min, 7 L/min, 7.5 L/min, 8 L/min, 8.5 L/min, 9 L/min, 9.5 L/min, 10 L/min, etc.); separating the clot material from blood by capturing the clot material into a chamber of the blood removal and return circuit; and returning filtered blood from the blood collection chamber to the patient.

Clause 145. An aspiration device for use with an aspiration catheter, said aspiration device comprising: a chamber having a pressure port, a blood inlet port, and a blood outlet port; a pump having a positive pressure port and a negative pressure port; a valve configured to selectively connect the positive and negative pressure ports of the pump to the pressure port of the chamber; a controller configured to control the valve to selectively apply negative and positive pressure from the pump to an interior of the chamber to draw blood into the chamber interior through the blood inlet port and to deliver blood from the interior through the blood outlet port.

Clause 146. The aspiration device of Clause 145, wherein the pressure port is located on an upper region of the chamber and the blood inlet and blood outlet ports are located on a lower region of the chamber.

Clause 147. The aspiration device of Clause 146, wherein blood inlet and blood outlet ports each comprise a one-way flow element to control fluid flow direction.

Clause 148. The aspiration device of Clause 145 or 147, wherein the chamber pressure port comprises a float valve to prevent blood from being extracted by the pump.

Clause 149. A method of closed-loop clot removal and blood return using a blood removal and return circuit coupled to a patient, the method comprising: applying aspiration from an aspiration orifice by generating on-demand negative pressure from a stored positive pressure, to draw the clot material and blood into the blood removal and return circuit; applying positive pressure to move the blood and clot material into a visualization chamber of the blood removal and return circuit; separating and visualizing the clot material in the visualization chamber of the blood removal and return circuit; and returning the blood to the patient.

Clause 150. A method of closed-loop clot removal and blood return using a blood removal and return circuit coupled to a patient, the method comprising: outputting an indicator that a clot material is proximate to an aspiration orifice; applying a pulse of aspiration from the aspiration orifice by generating on-demand negative pressure from a stored positive pressure, to draw the clot material into the blood removal and return circuit; separating the clot material from blood by capturing the clot material into a chamber of the blood removal and return circuit; filtering the blood to remove the clot material; and returning filtered blood to the patient.

Clause 151. A method of closed-loop clot removal and blood return using a blood removal and return circuit coupled to a patient, the method comprising: applying aspiration from an aspiration orifice by generating on-demand negative pressure to draw the clot material and blood into the blood removal and return circuit; moving the blood and clot material into a visualization chamber of the blood removal and return circuit; separating and visualizing the clot material in the visualization chamber of the blood removal and return circuit; and returning the blood to the patient.

Clause 152. A method of closed-loop clot removal and blood return using a blood removal and return circuit coupled to a patient, the method comprising: outputting an indicator that a clot material is proximate to an aspiration orifice; applying a pulse of aspiration from the aspiration orifice by generating on-demand negative pressure to draw the clot material into the blood removal and return circuit; separating the clot material from blood by capturing the clot material into a chamber of the blood removal and return circuit; filtering the blood to remove the clot material; and returning filtered blood to the patient.

Clause 153. An aspiration device, the device comprising: an aspirator comprising an aspirator plunger within a tubular body and an aspirator port at one end region of the tubular body configured to couple to an aspiration catheter; and a fluidic driver including a driver displacement plunger within a tubular driver body, and at least a first fluid port configured to receive a pressurized drive fluid, wherein the pressurized fluid causes the driver displacement plunger to translate in a first direction and wherein the aspirator plunger is coupled to travel in tandem with the driver displacement element to aspirate blood and clot from the aspiration catheter.

Clause 154. An aspiration device, the device comprising: an aspirator comprising an aspirator plunger within a tubular body and an aspirator port at one end region of the tubular body configured to couple to an aspiration catheter; and a fluidic driver including a driver displacement plunger movably disposed within a tubular driver body and partitioning the tubular driver body into a first region and a second region, and a first fluid port configured to pass a first pressurized drive fluid into the first region and a second fluid port configured to pass a second pressurized drive fluid into the second region, wherein a pressure differential between the first region and the second region causes the driver displacement plunger to translate in the tubular driver body and wherein the aspirator plunger is coupled to travel in tandem with the driver displacement element to aspirate blood and clot through the aspirator port and into and from the tubular body when a distal port of the aspiration catheter is in a patient blood vessel proximate clot.

Clause 155. A system, the system comprising: a source of pressurized fluid; an aspirator comprising an aspirator plunger within a tubular body and an aspirator port at one end region of the tubular body configured to couple to an aspiration catheter; and a fluidic driver including a driver displacement plunger within a tubular driver body, and at least a first fluid port configured to receive a pressurized fluid, wherein the aspirator plunger is coupled to travel in tandem with the driver displacement element; a valve assembly configured to selectively deliver pressurized fluid into the tubular driver body; and a controller coupled to the valve assembly and configured to control the application of pressurized fluid into the tubular driver body to adjust the position and/or rate of movement of the driver displacement plunger within the tubular driver body based on a user input.

Clause 156. A method for aspirating clot from the vasculature of a patient, said method comprising: positioning a distal port of an aspiration catheter proximate the clot in the patient's vasculature, wherein the aspiration catheter is coupled to an aspirator port of an aspirator including an aspirator plunger within a tubular body; and delivering a pressurized drive fluid to tubular driver body of a fluidic driver, wherein the fluidic driver comprises a driver displacement plunger within the tubular driver body, further wherein the driver displacement plunger is coupled to the aspirator plunger; wherein delivery of the pressurized fluid to the first port translates the driver displacement plunger in a first direction which translates the aspirator plunger and generates a negative pressure in the tubular body of the aspirator to draw the clot and blood into the aspiration catheter.

Clause 157. A method of closed-loop clot removal and blood return using a blood removal and return circuit coupled to a patient, the method comprising: applying aspiration from an aspiration orifice by generating on-demand negative pressure to draw the clot material into the blood removal and return circuit; separating the clot material from blood by capturing the clot material into a visualization chamber of the blood removal and return circuit; separating and visualizing the clot material in the visualization chamber of the blood removal and return circuit; opening the visualization chamber to remove the clot material without breaking the blood removal and return circuit; directing the blood into a blood collection chamber that is configured to de-air the blood; and returning de-aired blood from the blood collection chamber to the patient.

Clause 158. A blood filtering apparatus, the apparatus comprising: a filter chamber having a filter that divides the chamber into an upper portion having a blood inlet and a lower portion, wherein the filter element is configured to separate clot from a pressurized flow of blood and clot entering the upper portion through the blood inlet and pass blood substantially free from clot into the lower portion; a deaeration chamber having a lower portion including a blood outlet and an upper portion including a gas vent, wherein the lower portion of the deaeration chamber is configured to receive filtered, pressurized blood from the lower portion of the filter chamber and to separate gas present in said filtered, pressurized blood and the upper portion is configured to allow the separated gas to pass out through the gas vent; and a one-way valve configured to allow pressurized, filtered blood in the lower portion of the filter chamber to flow the lower portion of the deaeration chamber and to prevent a reverse flow of blood from the deaeration chamber to the filter chamber.

Clause 159. A method for filtering clot from blood, said method comprising: pressurizing blood having entrained clot to cause the blood to sequentially flow through: (a) a filter chamber wherein clot separates on an upper surface of a filter and filtered blood substantially free from clot collects in a lower portion of the filter chamber; and (b) a deaeration chamber wherein gas present in said filtered blood separates and collects in an upper portion of the deaeration chamber and passes out through a gas vent.

Clause 160. A method of closed-loop clot removal and blood return using a blood removal and return circuit coupled to a patient, the method comprising: applying aspiration from an aspiration orifice and through a first portion of the blood removal and return circuit during a withdrawal stroke of a pump cycle by generating a pulse of negative pressure to draw the clot material and blood into the blood removal and return circuit; and moving blood into a blood collection chamber within a second portion of the blood removal and return circuit during a return stroke of the pump cycle by applying positive pressure, wherein the second portion of the blood removal and return circuit is closed during the withdrawal stroke and wherein the first portion of the blood removal and return circuit is closed during the return stroke; and returning blood from the blood collection chamber to the patient.

Clause 161. A method of closed-loop clot removal and blood return using a blood removal and return circuit coupled to a patient, the method comprising: applying aspiration from an aspiration orifice and through a first portion of the blood removal and return circuit during by generating a pulse of negative pressure to draw the clot material and blood into the blood removal and return circuit; and applying positive pressure through the blood removal and return circuit to drive the clot material and blood into a clot collection chamber within a second portion of the blood removal and return circuit to generate a positive air pressure within the clot collection chamber; filtering the blood within the clot collection chamber and driving the filtered blood; into a blood de-airing chamber to de-air the blood, wherein the blood is moved from the clot collection chamber to the de-airing chamber by the positive air pressure; and returning blood from the blood collection chamber to the patient.

Clause 162. A method of closed-loop clot removal and blood return using a blood removal and return circuit coupled to a patient, the method comprising: withdrawing, filtering and return blood to the patient by repeating the steps of: applying aspiration from an aspiration orifice and through a first portion of the blood removal and return circuit during a withdrawal stroke of a displacement pump by generating a pulse of negative pressure to draw the clot material and blood into the blood removal and return circuit; and moving blood into a blood collection chamber within a second portion of the blood removal and return circuit during a return stroke of the displacement pump cycle by applying positive pressure, wherein the second portion of the blood removal and return circuit is closed during the withdrawal stroke and wherein the first portion of the blood removal and return circuit is closed during the return stroke; and returning blood from the blood collection chamber to the patient during the return stroke, wherein the displacement pump is driven by positive pressure.

Clause 163. A method of closed-loop clot removal and blood return using a blood removal and return circuit coupled to a patient, the method comprising: applying aspiration from an aspiration orifice by generating on-demand negative pressure to draw the clot material into the blood removal and return circuit; separating the clot material from blood by capturing the clot material into a chamber of the blood removal and return circuit; directing the blood into a blood collection chamber that is configured to de-air the blood; and returning de-aired blood from the blood collection chamber to the patient.

Clause 164. A method of closed-loop clot removal and blood return using a blood removal and return circuit coupled to a patient, the method comprising: applying aspiration from an aspiration orifice by generating on-demand negative pressure to draw the clot material into the blood removal and return circuit; separating the clot material from blood by capturing the clot material into a chamber of the blood removal and return circuit; directing the blood into a blood collection chamber that is configured to de-air the blood; de-foaming the blood in the blood collection chamber using a foam separator; and returning de-aired blood from the blood collection chamber to the patient.

Clause 165. The method of Clause 164, further comprising reducing foaming using a foam separator in the de-airing chamber.

Clause 166. The method of Clause 164, wherein separating the clot material from blood comprises capturing the clot material in a visualization chamber of the blood removal and return circuit.

Clause 167. The method of Clause 164, further comprising sensing clot material proximal to an aspiration orifice of an aspiration catheter.

Clause 168. Blood filtering apparatus comprising: a filter chamber having a filter element that divides the chamber into an upper portion having a blood inlet and a lower portion, wherein the filter element is configured to separate clot from a pressurized flow of blood and clot entering the upper portion through the blood inlet and pass blood substantially free from clot into the lower portion; a deaeration chamber having a lower portion including a blood outlet and an upper portion including a gas vent, wherein the lower portion of the deaeration chamber is configured to receive filtered, pressurized blood from the lower portion of the filter chamber and to separate gas present in said filtered, pressurized blood and the upper portion is configured to allow the separated gas to pass out through the gas vent; and a one-way valve configured to allow pressurized, filtered blood in the lower portion of the filter chamber to flow the lower portion of the deaeration chamber and to prevent a reverse flow of blood from the deaeration chamber to the filter chamber.

Clause 169. The apparatus of Clause 168, wherein at least a portion of a top of the filter chamber is sufficiently transparent to allow viewing of clot collected on an upper surface of the filter.

Clause 170. The apparatus of any of Clauses 168-169, wherein the top is removable to allow removal and return of at least an upper portion of the filter element to permit cleaning of the clot.

Clause 171. The apparatus of any of Clauses 168-170, further comprising means for cleaning a lower surface of the top of the filter chamber to remove adherent clot and improve viewing.

Clause 172. The apparatus of Clause 168, wherein the means for cleaning a lower surface of the top of the filter chamber comprises a rotatable wiper blade.

Clause 173. The apparatus of any of Clauses 168-172, wherein at least a portion of the filter element is removable from the filter chamber to allow clot to be removed from an upper surface thereof.

Clause 174. The apparatus of any of Clauses 168-173 wherein the filter element comprises an upper strainer component and a lower microporous filter component, wherein the upper strainer component is removably positioned over the lower microporous filter component.

Clause 175. The apparatus of any of Clauses 168-174 wherein the upper strainer component is separable from the lower microporous filter component.

Clause 176. The apparatus of any of Clauses 168-175, wherein the lower microporous filter component is fixedly positioned within the filter chamber.

Clause 177. The apparatus of any of Clauses 168-176, wherein the gas vent on the upper portion of the deaeration chamber comprises a gas vent valve configured to close when the deaeration chamber fills with pressurized blood and to open when separated gas collects in the upper portion of the deaeration chamber.

Clause 178. The apparatus of Clause 177, wherein the gas vent valve comprises a float valve which is buoyed by blood in the deaeration chamber and opened by gas collecting in the upper portion of the deaeration chamber above the float valve.

Clause 179. The apparatus of Clause 178, wherein the float valve comprises a resilient seal on an upper surface thereof, wherein the resilient seal engages a vent port on an upper wall of the deaeration chamber.

Clause 180. The apparatus of Clause 178, wherein the float valve rides on rails disposed on an inner wall of the deaeration chamber.

Clause 181. The apparatus of any of Clauses 168-180, further comprising a vertical support tube having a deflector on an upper end thereof, wherein the vertical support tube is configured to receive the pressurized, filtered blood entering the lower portion of the deaeration chamber and to pass the blood upwardly to engage a lower surface of the deflector which redirects the blood downwardly and allows gas to separate and rise upwardly into the upper portion of the deaeration chamber.

Clause 182. The apparatus of any of Clauses 168-181, further comprising a cutoff valve at the blood outlet of the deaeration chamber, wherein the cutoff valve is configured to close the blood outlet if blood in the deaeration chamber falls below a minimum level.

Clause 183. The apparatus of Clause 182, wherein the cutoff valve comprises a ball valve.

Clause 184. The apparatus of any of Clauses 168-183, further comprising a pressure source connectable to a proximal end of an aspiration catheter and to the lower portion of the filter chamber, wherein said pressure source is configured generate a negative pressure to draw blood and clot from the aspiration catheter and to generate a positive pressure to transfer the drawn blood and clot to the lower portion of the filter chamber.

Clause 185. The apparatus of any of Clauses 168-184, wherein the pressure source comprises a piston pump configured to apply the negative pressure by retracting a piston and to apply the positive pressure by advancing the piston.

Clause 186. The method of any of Clauses 168-185, wherein pressure source comprises a syringe configured to apply the negative pressure by retracting a plunger of the syringe and to apply the positive pressure by advancing the plunger of the syringe.

Clause 187. A method for filtering clot from blood, said method comprising: pressurizing blood having entrained clot to cause the blood to sequentially flow through: (a) a filter chamber wherein clot separates on an upper surface of a filter element and filtered blood substantially free from clot collects in a lower portion of the filter chamber; and (b) a deaeration chamber wherein gas present in said filtered blood separates and collects in an upper portion of the deaeration chamber and passes out through a gas vent.

Clause 188. The method of Clause 187, wherein the blood having entrained clot is pressurized with a piston pump.

Clause 189. The method of Clause 188, wherein the piston pump comprises a syringe.

Clause 190. The method of Clause 187, wherein the blood is pressurized with a continuous pump.

Clause 191. The method of any of Clauses 187-190, further comprising viewing clot which has collected on the upper surface of the filter element though a transparent top of the filter chamber.

Clause 192. The method of Clause 191, further comprising stopping the blood pressurization, removing the transparent top, removing at least a portion of the filter element from the filter chamber, and cleaning clot from the removed at least a portion of the filter element.

Clause 193. The method of Clause 192, wherein an upper strainer portion of the filter element is removed while a lower microporous filter component remains in the filter chamber to minimize the risk of clot falling into filtered blood in the lower portion of the filter chamber.

Clause 194. The method of any of Clauses 187-193, wherein the filtered blood passes from the filter chamber to the deaeration chamber through a one-way valve that prevents backflow from the deaeration chamber to the filter chamber.

Clause 195. The method of any of Clauses 187-194, wherein the filtered blood passes from the lower portion of the filter chamber to a lower portion of the deaeration chamber.

Clause 196. The method of Clause 187, wherein the filtered blood flows upwardly from the lower portion of the deaeration chamber through a vertical tube and is released into the upper portion of the deaeration chamber wherein gas separates from the filtered blood and collects at the top of the deaeration chamber and wherein the filtered blood collects at the bottom of the deaeration chamber.

Clause 197. The method of any of Clauses 187-196, wherein gas flow through the vent valve is controlled by a float valve.

Clause 198. The method of any of Clauses 187-197, wherein the filtered blood released from the vertical tube engages a lower surface of a deflector that directs the filtered blood flow downwardly and allows the separated gases to pass upwardly.

Clause 199. The method of Clause 198, wherein the float valve is disposed over an upper surface of the deflector and rises to seal against a vent port when the deaeration chamber fills with blood and falls to open the vent port in response to gas collecting in the upper portion of the deaeration chamber.

Clause 200. The method of any of any of Clauses 187-199, wherein the pressurizing blood step comprises: aspirating the blood entrained with clot from a patient through an aspiration catheter using a pressure source to apply a negative pressure to the aspiration catheter; and using the same pressure source to apply a positive pressure to pressurize the blood and entrained clot to cause the aspirated blood entrained with clot to flow into the filter chamber.

Clause 201. The method of Clause 200, wherein the pressure source comprises a piston pump and applying the negative pressure comprises retracting a piston of the piston pump and applying the positive pressure comprises advancing the piston of the piston pump.

Clause 202. The method of any of Clauses any of Clauses 187-201, wherein pressure source comprises a syringe and applying the negative pressure comprises retracting a plunger of the syringe and applying the positive pressure comprises advancing the plunger of the syringe.

Clause 203. The method of any of Clauses any of Clauses 187-202, further comprising returning filtered blood from the deaeration chamber to the patient.

Clause 204. The method of Clause 203, wherein the filtered blood is returned to the patient through an access sheath used to introduce the aspiration catheter.

Clause 205. An aspiration system comprising: an aspiration catheter; an aspirator configured to connect to a proximal end of the aspiration catheter and to generate a negative pressure to aspirate blood and clot into an aspiration lumen of the aspiration catheter: a first pressure sensor coupled to a proximal end of the aspiration catheter; a second pressure sensor coupled to an inlet of the aspirator; and control circuitry configured to receive pressure measurements from the first and second pressure sensors and to detect clogging based upon the pressure measurements.

Clause 206. The aspiration system of Clause 205, wherein a pressure detected by the first pressure sensor being lower than expected indicates a blockage in the aspiration catheter.

Clause 207. The aspiration system of Clause 205, wherein a pressure detected by the second pressure sensor being lower than expected indicates a blockage in a line connecting the aspiration catheter to the aspirator.

Clause 208. An aspiration system comprising: an aspiration catheter; an aspirator configured to connect to a proximal end of the aspiration catheter and to generate a negative pressure to aspirate blood and clot into an aspiration lumen of the aspiration catheter: a first pressure sensor coupled to a proximal end of the aspiration catheter; a second pressure sensor coupled to an inlet of the aspirator; and control circuitry configured to receive pressure measurements from the first and second pressure sensors and to detect clogging based upon the pressure measurements.

Clause 209. A system for use with an aspiration catheter and a blood return cannula, said system comprising: a filter chamber having an inlet, an outlet, and a filter element therein that divides the chamber into a clot collecting portion and a blood transfer portion; a first pressure source connectable to a proximal end of the aspiration catheter and to the inlet of the filter chamber, wherein said first pressure source is configured generate a negative pressure to draw clot and blood from the aspiration catheter and to generate a positive pressure to transfer the drawn blood and clot to the inlet of the filter chamber; and a second pressure source configured to fill with blood from the blood transfer portion of the filter chamber as the first pressure source generates positive pressure, wherein the second pressure source is further configured to generate a positive pressure to deliver the filtered to the blood return cannula.

Clause 210. The system of Clause 209, wherein the amount of clot collected in the clot collecting portion is externally visible.

Clause 211. The system of any of Clauses 209-210, wherein the filter is removable from the filter chamber to allow clot to be removed from the filter element when the chamber is not pressurized.

Clause 212. The system of any of Clauses 209-211, wherein first pressure source comprises a syringe.

Clause 213. The system of any of Clauses 209-212, wherein second pressure source comprises a syringe.

Clause 214. The system of any of Clauses 209-213, wherein the filter chamber has a vertical dimension, and the filter element is oriented horizontally.

Clause 215. A method for clot aspiration and blood return, said method comprising: aspirating blood and clot from the vasculature of a patient into an inlet of a filter chamber having a filter element therein, wherein the clot collects on a surface of the filter and the blood passes to a receptacle; and separately pressurizing the receptacle to return the filtered blood to the patient.

Clause 216. The method of Clause 215, wherein aspirating the blood and clot comprises applying a negative pressure from a first pressure source to an aspiration catheter in the patient's vasculature and thereafter applying a positive pressure from the first pressure source to transfer the blood and clot to the filter chamber.

Clause 217. The method of Clause 216, wherein the first pressure source comprises a first syringe and applying the negative pressure comprises retracing a plunger of the syringe and applying the positive pressure comprises advancing the plunger of the syringe.

Clause 218. The method of any of Clauses 215-217, wherein separately pressurizing the receptacle to return the filtered blood to the patient comprises applying a positive pressure from a second pressure source to the receptacle.

Clause 219. The method of Clause 218, wherein the second pressure source comprises a second syringe and applying the positive pressure comprises advancing a plunger of the second syringe.

Clause 220. The method of Clause 219, wherein the blood and clot are aspirated by a first blood pump and the receptacle comprises a second blood pump.

Clause 221. The method of Clause 220, wherein the second blood pump is actuated to return the filtered blood to the patient through the filter chamber.

Clause 222. The method of Clause 221, further comprising detaching the second blood pump from the filter, attaching the second blood pump to a separate blood return location in the patient's vasculature, and actuating the second blood pump to return the filtered blood to the patient.

Clause 223. A system for use with an aspiration catheter and a blood return cannula, said system comprising: a filter chamber having an inlet, an outlet, and a filter element therein that divides the chamber into a clot collecting portion and a blood transfer portion; at least a first pressure source connectable to a proximal end of the aspiration catheter and to the inlet of the filter chamber, wherein said first pressure source is configured generate a negative pressure to draw clot and blood from the aspiration catheter and to generate a positive pressure to transfer the drawn blood and clot to the inlet of the filter chamber; wherein the blood transfer portion of the filter chamber is configured to return filtered blood to the blood return cannula; and wherein the amount of clot collected in the clot collecting portion is externally visible and the filter is removable from the filter chamber to allow clot to be removed from the filter element when the chamber is not pressurized.

Clause 224. The system of Clause 223, further comprising a second pressure source configured to fill with blood from the blood transfer portion of the filter chamber as the first pressure source generates positive pressure, wherein the second pressure source is further configured to generate a positive pressure to deliver the filtered to the blood return cannula.

Clause 225. The system of any of Clauses 223-224, wherein first pressure source comprises a syringe.

Clause 226. The system of any of Clauses 223-225, wherein second pressure source comprises a syringe.

Clause 227. The system of any of Clauses 223-226, wherein the filter chamber has a vertical dimension and the filter element is oriented horizontally, wherein a top of the filter chamber is removable to allow the filter element to be lifted to remove accumulated clot while leaving the filtered blood in the blood transfer portion.

Clause 228. A method for clot aspiration and blood return, said method comprising: aspirating blood and clot from the vasculature of a patient into an inlet of a filter chamber having a filter element therein, wherein the clot collects on a surface of the filter and the blood passes to a receptacle; and removing the filter from the chamber and cleaning the removed filter when excess clot has collected on the filter surface.

Clause 229. The method of Clause 228, wherein aspirating the blood and clot comprises applying a negative pressure from a first pressure source to an aspiration catheter in the patient's vasculature and thereafter applying a positive pressure from the first pressure source to transfer the blood and clot to the filter chamber.

Clause 230. The method of Clause 229, wherein the first pressure source comprises a first syringe and applying the negative pressure comprises retracting a plunger of the syringe and applying the positive pressure comprises advancing the plunger of the syringe.

Clause 231. The method of any of Clauses 228-229, wherein separately pressurizing the receptacle to return the filtered blood to the patient comprises applying a positive pressure from a second pressure source to the receptacle.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

One of skill in the art will realize that many variations on the disclosed embodiments are possible while staying within the bounds of the disclosed technologies as defined in the claims hereinbelow. Solely by way of example, variations in the design and function of the specific aspiration catheters, the filtration chambers, the deaeration chambers, the fluidic drivers, the fluidic actuators, the aspirators, and other particular components of the disclosed technologies described herein will fall within the claims herein. The embodiments provided are representative in nature and not meant to be limiting.

1. A method of clot removal, the method comprising:
positioning an aspiration catheter adjacent to a first clot material within a patient;
actuating a pressure source one or more times by triggering a control, wherein each actuation of the pressure source: aspirates the first clot material and blood, filters the first clot material from the blood, and drives the filtered blood into a collection chamber;
repositioning the aspiration catheter adjacent to a second clot material within the patient; and
actuating the pressure source one or more times, wherein each actuation of the pressure source: aspirates the second clot material and blood, filters the clot material from blood, and drives filtered blood to the collection chamber.

2. The method of claim 1, wherein each actuation comprises a first stroke and a second stroke.

3. The method of claim 2, wherein the first stroke comprises a first movement of a piston of the pressure source and wherein the second stroke comprises a return movement of the piston of the pressure source.

4. The method of claim 2, wherein the first stroke comprises a negative pressure stroke and the second stroke comprises a positive pressure stroke.

5. The method of claim 2, wherein the first stroke results in a first flow rate by which clot material is aspirated with blood and wherein the second stroke results in a second flow rate by which filtered blood is driven into the collection chamber.

6. The method of claim 5, wherein the first flow rate is greater than the second flow rate.

7. The method of claim 1, further comprising sensing that the aspiration catheter is adjacent to the first clot material using one or more sensors on the aspiration catheter.

8. The method of claim 7, wherein sensing comprises sensing an electrical signal.

9. The method of claim 1, wherein each actuation of the pressure source comprises operating a control in communication with the pressure source.

10. The method of claim 9, wherein operating the control comprises pushing a button.

11. The method of claim 1, wherein actuating the pressure source comprises (a) aspirating blooding and clot from the aspiration catheter and (b) collecting filtered blood in a reservoir in one step and driving filtered blood from the reservoir to the collection chamber in another step.

12. The method of claim 1, wherein aspirating and filtering the blood are performed at a first flow rate and driving the filtered blood to the collection chamber is performed at a second flow rate slower than the first flow rate.

13. The method of claim 1, wherein actuating the pressure source is automatically actuated by a controller.

14. The method of claim 13, wherein the controller is programmed to both (a) aspirate blood and clot from the aspiration catheter and (b) collect filtered blood in a reservoir in one step and to drive filtered blood from the reservoir to the collection chamber in another step.

15. The method of claim 14, wherein the controller is further programmed to aspirate and filter the blood at a first flow rate and drive the filtered blood to the collection chamber at a second flow rate slower than the first flow rate.

16. A method of clot removal, the method comprising:
positioning an aspiration catheter adjacent to a first clot material within a patient;
actuating a control to trigger a pressure source, wherein each actuation of the control causes the pressure source to: aspirate the first clot material, filter the first clot material from blood, and drive filtered blood to a collection chamber;
repositioning the aspiration catheter to be adjacent to a second clot material within the patient; and
actuating the control to trigger the pressure source, wherein each actuation causes the pressure source to: aspirate the second clot material, filter the second clot material from blood, and drive the filtered blood to the collection chamber.

17. The method of claim 16, wherein each actuation causes the pressure source to deliver a first stroke and a second stroke.

18. The method of claim 17, wherein the first stroke comprises a negative pressure stroke and the second stroke comprises a positive pressure stroke.

19. The method of claim 17, wherein the first stroke results in a first flow rate by which clot material is aspirated with blood and wherein the second stroke results in a second flow rate by which filtered blood is driven to the collection chamber.

20. The method of claim 19, wherein the first flow rate is greater than the second flow rate.

21. The method of claim 16, further comprising sensing, using one or more sensors on the aspiration catheter, that the aspiration catheter is adjacent to the first clot material.

22. The method of claim 21, wherein sensing comprises sensing an electrical impedance.

23. The method of claim 16, wherein actuating the control comprises pushing a button.

24. A method of clot removal, the method comprising:
positioning an aspiration catheter adjacent to a first clot material within a patient;
actuating a control to trigger a pressure source, wherein each actuation of the control causes the pressure source to generate a negative pressure stroke that aspirates the first clot material and a positive pressure stroke that drives filtered blood to a collection chamber, wherein the blood is filtered during the negative pressure stroke and/or the positive pressure stroke;
repositioning the aspiration catheter to be adjacent to a second clot material within the patient; and
actuating the control to trigger the pressure source, wherein each actuation causes the pressure source to generate the negative pressure stroke that aspirates the second clot material and the positive pressure stroke that drives filtered blood to the collection chamber, wherein the blood is filtered during the negative pressure stroke and/or the positive pressure stroke.

25. The method of claim 24, wherein the negative pressure stroke results in a first flow rate by which clot material is aspirated with blood and wherein the positive pressure stroke results in a second flow rate by which filtered blood is driven to the collection chamber.

26. The method of claim 25, wherein the first flow rate is greater than the second flow rate.

27. The method of claim 24, further comprising sensing, using one or more sensors on the aspiration catheter, that the aspiration catheter is adjacent to the first clot material.

28. The method of claim 27, wherein sensing comprises sensing an electrical impedance.
